(19) United States
(12) Patent Application Publication
(10) Pub. No.: US 2024/0252067 A1
(43) Pub. Date: Aug. 1, 2024

(54) SYSTEMS, DEVICES, AND METHODS FOR WELLNESS MONITORING WITH PHYSIOLOGICAL SENSORS

(71) Applicant: ABBOTT DIABETES CARE INC., Alameda, CA (US)

(72) Inventors: Junli Ou, Pleasanton, CA (US); James P. McCarter, St. Louis, MO (US); Justin N. Williams, Oakland, CA (US); Olivier Ropars, Kirkland, WA (US); Ismene Grohmann, San Francisco, CA (US)

(21) Appl. No.: 18/406,847

(22) Filed: Jan. 8, 2024

Related U.S. Application Data
(60) Provisional application No. 63/527,626, filed on Jul. 19, 2023.
(60) Provisional application No. 63/445,653, filed on Feb. 14, 2023.
(60) Provisional application No. 63/438,218, filed on Jan. 10, 2023.

(51) Int. Cl. A61B 5/145; A61B 5/00; G16H 10/20

(52) U.S. Cl. CPC A61B 5/14532; A61B 5/0004; A61B 5/7435; A61B 5/7475; G16H 10/20

(57) ABSTRACT

Systems and methods for displaying metrics related to a user are described. Data indicative of glucose levels of the user is received. A first alert point for a potential glucose episode in a data set of time correlated glucose data, e.g., a glucose vs. time curve, is identified if the last received glucose data point satisfies at least one alert condition. A first potential local minimum in a first time period is identified. The first potential local minimum is confirmed as a first start point of a first glucose episode if the first potential local minimum satisfies at least one local minimum condition. An integrated area under the curve over time of a first portion of the graph beginning at the first start point of the first glucose episode to the first alert point is calculated. A first count value is assigned to the first portion.

### CROSS-REFERENCE TO RELATED APPLICATIONS

This application claims priority to U.S. Provisional Application No. 63/438,218, filed Jan. 10, 2023, U.S. Provisional Application No. 63/445,653, filed Feb. 14, 2023, and U.S. Provisional Application No. 63/527,626, filed Jul. 19, 2023, which are all herein expressly incorporated by reference in their entireties for all purposes.

### FIELD

The subject matter described herein relates generally to digital interfaces and user interfaces for analyte monitoring systems, as well as systems, methods, and devices relating thereto.

### BACKGROUND

The monitoring and management of wellness and nutrition in individuals can significantly benefit those at risk of or currently experiencing chronic health problems and those motivated to improve general wellness. These efforts can create several health and economic benefits for the individual, as well as the public at large. According to the CDC, for example, seven out of ten deaths in the United States occur each year from chronic diseases, and almost one out of every two adults has at least one chronic illness. Likewise, almost one in three children in the United States is overweight or obese, which predisposes them to chronic diseases. Many of these chronic diseases are preventable, or can be successfully treated if diagnosed at an early stage. In this regard, the monitoring and management of an individual's wellness and nutrition can significantly reduce the chance of chronic disease and, as a result, can mitigate future healthcare costs. Additional benefits of wellness and nutrition monitoring can further include enhancing athletic performing either during training, recovery, or during an athletic event.

To promote these goals, wearable technology can be utilized. A compact electronic device, for example, may be worn on the body, such as around the wrist, for monitoring an individual's heart rate or physical activity levels. Because physician visits are episodic (e.g., once per year), wearable technology can serve a useful function in providing timely physiological information to an individual, without the need for a physician visit, and which can ultimately lead to improved wellness. Despite these advantages, however, many people are reluctant to use wearable technology for various reasons, including the complexity of the data presented, a learning curve associated with using the wearable device, and inaccuracies with respect to the data. Some recent studies, for example, claim that existing wearable devices do not accurately measure an individual's heart rate or the number of calories burned.

Sensor control devices have been used by patients suffering from diabetes for many years. Many advances in these in vivo analyte monitoring systems have been made to increase comfort and convenience for the individual. Sensor control devices may have a small form-factor and can be applied by the individual with a sensor applicator. The application process includes inserting at least a portion of a sensor that senses a user's analyte level in a bodily fluid located in a layer of the human body, using an applicator or insertion mechanism, such that the sensor comes into contact with the bodily fluid. The benefits of analyte monitoring systems are not limited to persons with diabetes. For instance, analyte monitoring systems can provide useful information and insights to individuals interested in improving their health and wellness. As one example, to improve their sports performance, athletes can utilize a sensor control device worn on the body to collect data relating to one or more analytes such as, for example, glucose and/or lactate.

As sensor control devices that measure in vivo analyte levels have become more convenient, comfortable, and affordable for users, applications outside of medicine have become feasible. However, some existing user interfaces for sensor control devices are designed for medical use by patients under care of a physician, and not for non-medical applications such as, for example, athletic training and competition. As such, the data collected by the sensor control device, and methods for presenting the data to the user, may be unsuitable for non-medical applications. In addition, sensor control devices for non-medical (e.g., wellness and fitness) use may be confused with similar devices made for medical use, leading to problems in interpreting or using data.

Various applications make use of sensor data to perform various functions, including wellness functions. Thus, there exists a need to provide a framework that can communicate with physiological sensors and receive analyte data for use by various applications, including third party applications, but avoids the need for regulatory approval for every use-case for the data. Moreover, a need exists for digital interfaces and graphical user interfaces for analyte monitoring systems for medical and/or non-medical use, as well as methods and devices relating thereto, that are robust, user-friendly, and provide for timely and actionable responses.

Moreover, postprandial hyperglycemia, glucose spikes or glucose excursions following meals, is associated with increased hunger, decreased mood and energy for daily activities, disrupted sleep, weight gain and deleterious long-term health outcomes. A simplified method for users to track their glucose spikes with the goal of long-term reduction in glycemic (glucose) exposure is needed. Thus, needs exist for systems, devices and methods for wellness and nutritional monitoring and management that are more accurate and simpler to use by an individual.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter may be directed to a software library for use by applications to obtain sensor data. The software library can include a sensor control module, a remote management module, and include software logic for communication with a plurality of physiological sensors and applications. The sensor control module can authenticate the receiving device to allow the receiving device to receive sensor data, including by enabling communication with each of the plurality of physiological sensors to receive sensor data including data indicative of a different physiological signal. The sensor control module can further store the sensor data in a memory of the computing device. The sensor control module can obtain an output indicative of the different physiological signals from the sensor data of each of the plurality of physiological sensors. The sensor control module can provide the output of the different physiological signals from the physiological sensors to the authenticated third-party application running on the computing device.

In accordance with the disclosed subject matter, the physiological sensors can comprise an analyte sensor configured to detect an analyte level in a bodily fluid of a user. The output of the different physiological signals can also comprise an analyte value. The output can further comprise a notification of a physiological condition. The output can further indicate information about delivery of a medicament to a user.

In accordance with the disclosed subject matter, the communication session within the computing device and between the computing device and the physiological sensors can comprise a near-field communication (NFC), Bluetooth low energy (BLE), or any suitable wireless communication protocol known in the art.

The software library can further include a remote data management module including instructions to transmit sensor data to a remote server over a network. The remote management module can be configured to communicate with the remote server to authenticate the sensor control module, third-party application, or any other application. The authentication can use a uniform user interface irrespective of the application accessing the software library.

In accordance with the disclosed subject matter, the plurality of physiological sensors and the software library are subject to regulatory approval, including as software as a medical device. The output indicative of the physiological signal from the physiological sensors is also subject to regulatory approval. However, the third-party application running on the computing device is not subject to regulatory approval.

The software library can be configured to be implemented as a component of the authenticated third-party application. Because of the modular architecture and shared functionality, sensor data can be substantially simultaneously received, interpreted, and displayed from a plurality of physiological sensors.

The systems and methods described herein include a simplified user experience for tracking glucose exposure that is based on an algorithm that identifies glucose spikes, e.g., a rapid and sustained increases in glucose level, and tracks the spike as it progresses including defining spike start and end. A value (e.g., count) may be assigned to the spike as it is occurring or after it has terminated. The count may be updated through spike progression if the count is being assigned in real time as it is occurring. As described herein, glucose spikes, glucose peaks, and glucose excursions may be used interchangeably.

In some embodiments, the systems and methods described herein provide a wellness application that provides a way of tracking glucose exposure rather than providing the user with a glucose mean and measure of variance for a time period, such as a full day, week, or month. Such a system enables a user to focus on individual events, such as meals that are understandable for the user, rather than an aggregation of events, such as identified patterns based on past time periods, that may be less easily tied to specific choices or actions. Additionally, the application is able to provide information to the user about a current spike or current excursion status, enabling immediate action (e.g., a nudge to walk after a meal) and enabling learning in real-time (e.g., associating the contents or circumstances of a particular meal or snack with a specific spike response).

In some embodiments, the systems and methods described herein provide a daily count total or value. The daily count total provides the user with an easy metric of their day's progression and prior day's outcome for glucose exposure. An aim for the application is for the user to attempt to keep their daily count (accumulated daily score) below the target daily score. Thus, each day using the wellness application, as well as each week using the wellness application, has an objective in reducing glucose exposure that is easy to understand.

In some embodiments, the wellness application is always running passively when the application is open. The wellness application may not require the user to log their meals or other activities although logging may make the information more interpretable to the user and may further improve their engagement, experience, and learnings.

In some embodiments, the algorithm in the wellness application may only be based on glucose values alone. In such a case, a universal scale for count determination may be used and values may be compared between and among users. Data may also be compared longitudinally by the user over the course of their experience with the application. In other embodiments, the algorithm may consider glucose values, along with other user attributes such as age, biological sex, BMI.

In some embodiments, the application does not determine the underlying reason for the glucose spike, which may be cause by a variety of user events including, (1) food consumption with glucose entering the blood stream from digestion, (2) stress, with glucose entering the blood stream from liver release, (3) exercise, with glucose entering the blood stream from liver release, or (4) sensor artifacts with sensor readings increasing from temperature changes or other causes.

In some embodiments, the user may tag exercise such that the algorithm detected spikes due to exercise are ignored and associated counts with an exercise spike may be excluded from the daily counts total. Such a feature was created so that exercise, a healthy activity, is not penalized by the wellness application.

In some embodiments, the wellness application does not differentiate spikes or excursions and counts caused by food consumption versus stress. The user can add event tags to spikes for food consumption, exercise, or other (e.g., stress) but only in the case of exercise are spikes excluded. In other embodiments, the user can select an option to exclude counts associated with a stress spike from their daily total. In other embodiments, the wellness application may keep a separate tally or total of counts due to stress and other non-food associated activities and events. In other embodiments, the wellness application may include a filter in which the user may select which types of counts (food, exercise, stress, etc.) to include in a total count. In some embodiments, the user can select an option to exclude certain counts associated with food or stress from their daily total.

In accordance with the disclosed subject matter, a method for monitoring glucose variability includes a system that receives data indicative of glucose levels of the subject from a sensor control device is described. A first glucose variability metric of the subject may be determined in a first time period. The first glucose variability metric may then be compared to a threshold. A first indicator may be displayed if the first glucose variability metric does not exceed the threshold and a second indicator may be displayed if the first glucose variability metric exceeds the threshold.

The glucose variability metric may be variability with respect to a running baseline, a difference between a maximum and minimum glucose level, time in or out of a target range during the relevant time period, or a combination thereof.

In accordance with the disclosed subject matter, a method for monitoring glucose variability includes a system that receives data indicative of glucose levels of the subject from a sensor control device is described. A maximum glucose level and a minimum glucose level in a time period may be identified. A difference of the maximum glucose level and the minimum glucose level in the time period may be calculated. The difference may be compared to a threshold. A first indicator may be displayed if the difference does not exceed the threshold and a second indicator may be displayed if the difference exceeds the threshold.

In accordance with the disclosed subject matter, a system for determining and displaying metrics relating to a subject is described. The system includes one or more processors and a memory storing instructions that, when executed by the one or more processors, cause the system to: determine a glucose status of the subject based on glucose data received in a rolling window time period; display an indication of the glucose status of the subject in a graphic user interface (GUI), wherein the indication of the glucose status comprises a text description and a graphic having a first color; and display a graph in the GUI, wherein the graph comprises a glucose profile comprising a first portion and a second portion, wherein the first portion and second portion are different colors, and wherein the second portion is the first color.

In accordance with the disclosed subject matter, a system for determining and displaying metrics relating to a subject is described. The system includes one or more processors and a memory storing instructions that, when executed by the one or more processors, cause the system to: determine a plurality of glucose statuses of the subject based on glucose data received in a plurality of rolling window time periods, wherein each of the rolling window time periods comprises a logged activity; display a first graph comprising a first glucose profile for a first rolling window time period and a description of a first logged activity, wherein the first glucose profile comprises first, second, and third portions, wherein the first portion and third portions are a first color, and wherein the second portion is a second color, and display a second graph comprising a second glucose profile for a second rolling window time period and a description of a second logged activity, wherein the second glucose profile comprises first, second, and third portions, wherein the first portion and third portions are the first color, and wherein the second portion is a third color.

In accordance with the disclosed subject matter, systems and methods for determining and displaying a metric associated with glucose exposure is described.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of a system that includes a software library, receiving device, and sensor assembly.

FIG. 2 is a block diagram depicting an example embodiment of a receiving device.

FIG. 3 is a block diagram depicting an example embodiment of a sensor assembly.

FIG. 4 is a block diagram depicting an example software library, including a sensor control module and a remote management module, for communication with applications.

FIG. 5 is a block diagram depicting an example embodiment of the sensor control module.

FIG. 6 is a block diagram depicting an example embodiment of the remote management module.

FIG. 7A-7C are exemplary embodiments of user interfaces of applications using the inventive architecture.

FIGS. 8-9 are example methods for communicating sensor data from a sensor to an application or a third-party application using the disclosed subject matter.

FIGS. 10A-10E are example embodiments of GUIs related to a biosensor banner.

FIGS. 11A-11B are example embodiments of GUIs related to a biosensor module details.

FIGS. 12A-12B are example embodiments of GUIs related to system messages associated with a biosensor.

FIGS. 13A-13D are block diagrams depicting example embodiments of GUIs related to pairing a biosensor with a reader device.

FIG. 14 is a system overview of an analyte monitoring system comprising a sensor applicator, a sensor control device, a reader device, a network, a trusted computer system, and a local computer system.

FIG. 15A is a block diagram depicting an example embodiment of a reader device.

FIGS. 15B and 15C are block diagrams depicting example embodiments of sensor control devices.

FIGS. 16A-16B are example graphs depicting glucose exposure.

FIG. 17A-17C are flow diagrams depicting example embodiments of methods relating to a glucose count system for monitoring and managing an individual's glucose exposure.

FIGS. 18A-18D are flow diagrams depicting example embodiments of additional methods relating to a glucose count system for monitoring and managing an individual's glucose exposure.

FIG. 19A are exemplary graphs depicting glucose traces over time, glycemic counts, and count trends.

FIG. 19B is a block diagram depicting an exemplary GUI reflecting count trend status.

FIG. 20A is a flow diagram depicting an example embodiment for displaying a progress indicator.

FIGS. 20B-20D are exemplary block diagrams of live screens.

FIGS. 21A-21C are flow diagrams depicting example embodiments of methods relating to calculating a count trend status.

FIG. 22A is a flow diagram depicting an example embodiment of a method for determining a glucose profile.

FIG. 22B is an exemplary graph illustrating a sample calculation related to determining a glucose profile.

FIGS. 23A-23D are block diagrams depicting example embodiments of GUIs related to logging different activities, including food.

FIGS. 24A-24B are block diagrams depicting example embodiments of GUIs related to logging exercise.

FIGS. 25A-25C are block diagrams depicting exemplary GUIa of a daily report.

FIGS. 26A-26D are block diagrams depicting exemplary GUIs of weekly reports or portions of weekly reports.

FIG. 27 is a flow diagram depicting an example embodiment for determining counts for spikes related to food and non-food events.

FIG. 28 is a flow diagram depicting an example embodiment for determining target daily counts.

FIG. 29 is a block diagram depicting an exemplary GUI associated with a first stage of a glucose wellness application.

FIGS. 30A-30B are exemplary GUIs associated with a second stage of a glucose wellness application.

FIG. 31 is an exemplary GUI associated with a third stage of a glucose wellness application.

FIG. 32 is an exemplary GUI displaying a plurality of reports.

FIG. 33 is a flow diagram depicting an example embodiment for displaying glucose metrics.

### DETAILED DESCRIPTION

Reference will now be made in detail to the various exemplary embodiments of the disclosed subject matter, exemplary embodiments of which are illustrated in the accompanying drawings.

The system can include a device that receives analyte data measured by an analyte monitor and medication delivery data recorded by a delivery device, and processes and/or displays that data, in any number of forms, to the user. This device, and variations thereof, can be referred to as a “receiving device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. This device can be a smartphone, a smartwatch, or display device.

The system can also include an in vivo analyte monitor sensor assembly, which can comprise various types of monitors. For example, “Continuous Analyte Monitoring” systems (or “Continuous Glucose Monitoring” systems), can transmit data from a sensor device to a reader device continuously without prompting, e.g., automatically according to a schedule. “Flash Analyte Monitoring” systems (or “Flash Glucose Monitoring” systems or simply “Flash” systems), as another example, can transfer data from a sensor device in response to a scan or request for data by a reader device, such as with a Bluetooth Low-Energy (BLE), Near Field Communication (NFC) or Radio Frequency Identification (RFID) protocol. An in vivo analyte monitoring sensor assembly can also operate without the need for finger stick calibration.

In vivo monitoring sensor assemblies can include a sensor that, while positioned in vivo, contacts the bodily fluid of the user and generates analyte data indicative of the analyte levels contained therein. The sensor assembly can reside on the body of the user and contain the electronics and power supply that enable and control the analyte sensing. The sensor assembly, and variations thereof, can also be referred to as an “on-body electronics” device or unit, an “on-body”device or unit, or a “sensor data communication” device or unit, or analyte sensor, sensor device, in vivo analyte monitor sensor assembly, sensors, to name a few.

Further, the system can include an external device for use with the analyte sensor. For example and without limitation, external devices can include delivery devices that use information from the analyte sensor to determine or deliver amounts of a medication or other beneficial agents to a user. Additionally or alternatively, external devices can include other sensors, such as other analyte sensors, accelerometers, pressures sensors, or can include external computing devices, such as a medical server or a smartphone application configured to use analyte sensor information to provide additional insights to a user, including but not limited to insights related to medical conditions, well-being, fitness, appetite, or other medical or non-medical insights or analysis.

Generally, and as set forth in greater detail below, the disclosed subject matter provided herein includes a software library within a receiving device for communicating with analyte sensors and permitting third-party applications access to the sensor data for use in medically necessary applications or applications related to the well-being of the user. The system further includes a software library that can be implemented independently of the sensors and integrated within third-party applications to allow access to the sensor data. The sensor control module can further communicate with the sensor assemblies in such a manner to receive data simultaneously or substantially simultaneously from a plurality of such sensor assemblies. The system further enables the transfer of sensor information from the sensor control module to a remote management module.

The embodiments described herein can be used to monitor and/or process information regarding any number of one or more different analytes. Analytes that may be monitored include, but are not limited to, acetyl choline, amylase, bilirubin, cholesterol, chorionic gonadotropin, glycosylated hemoglobin (HbAlc), creatine kinase (e.g., CK-MB), creatine, creatinine, DNA, fructosamine, glucose, glucose derivatives, glutamine, growth hormones, hormones, ketones, ketone bodies (e.g., ß-hydroxybutyrate), lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In embodiments that monitor more than one analyte, the analytes may be monitored at the same or different times.

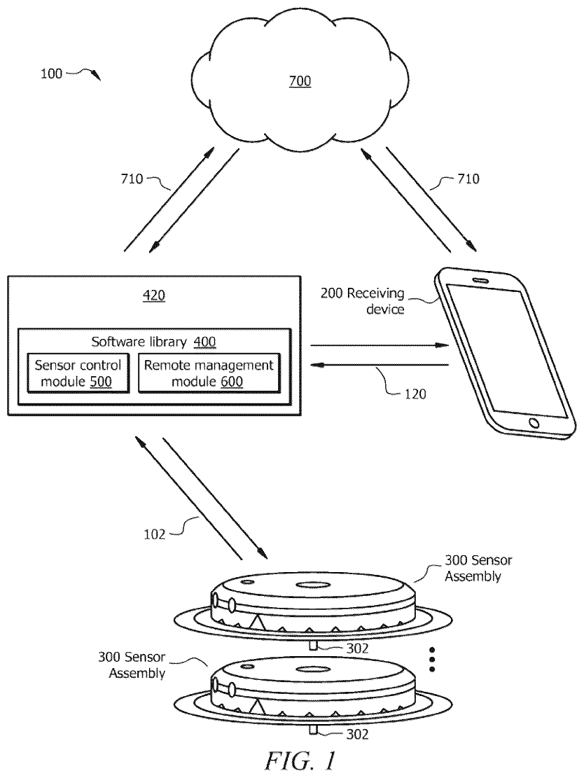

FIG. 1 is a schematic diagram depicting an example embodiment of a system 100 that includes a modular connectivity framework using a software library 400, various applications 420, a sensor assembly 300, and a receiving device 200.

In accordance with the disclosed subject matter, a non-transitory computer-readable storage medium includes a software library for use by applications 420 on a receiving device 200, or standalone devices such as a pump, insulin pen, etc., to obtain sensor data. The software library can include a sensor control module, a remote management module, and include software logic for communication with a plurality of sensors and applications. The sensor control module can authenticate the receiving device to allow the receiving device to receive sensor data, including by enabling communication with each of the plurality of sensors to receive sensor data including data indicative of a different signal. The sensor control module can further store the sensor data in a memory of the computing device. The sensor control module can obtain an output indicative of the different signals from the sensor data of each of the plurality of sensors. The sensor control module can provide the output of the different signals from the sensors to the authenticated third-party application running on the computing device.

The system 100 includes a software library 400 that functions using a modular architecture enabling a sensor control module 500 to communicate with and reside within various applications 420 on the receiving device 200. Applications 420 may further interface with sensor assembly 300 through the sensor control module 500, and in particular, by providing the request to the communication control module 540 (on FIG. 5) to interface directly with the sensor assembly 300. The sensor assembly 300 can also be one device with different sensors 302 or one sensor 302 configured to detect more than one analyte.

The receiving device 200 includes one or more applications 420, with each application instance embedding software library 400. The receiving device 200 uses a modular connectivity framework for the applications 420. In particular, the applications 420 each include a software library 400 including a remote management module 600 and sensor control module 500 for communicating with the one or more sensor assemblies 300. The software library 400 may also run as a service that executes simultaneously with the underlying application allowing the sensor control module 500 or remote management module 600 to execute as a service alongside one or more applications.

Sensor control module 500 may further interface with the sensor data. The various modules within the software library 400 implemented within the application 420 can send and receive communication with the sensor assembly 300 via communication link 102.

While the sensor control module 500 is within the application 420 in a receiving device 200, the sensor control module 500 could have base components in a second receiving device, such as a smartwatch, mobile device, or other wearable device. While such a device may not allow for a user interface experience that would be provided by a smartphone or tablet or computer, the smartwatch or wearable device can incorporate the sensor control module 500 to permit direct communication through the sensor control module 500 on the smartwatch or mobile wearable device with the sensor assembly 300. This would allow for applications specific to wearable devices to use sensor data. The wearable devices can synch separately with the receiving device 200, which can be used to perform the majority of the user login, initialization, authentication, and consent features to implement and initiate the receipt of sensor data.

Communication link 102 can be a wireless protocol including Bluetooth®, Bluetooth® Low Energy (BLE, BTLE, Bluetooth® SMART, etc.), Near-Field Communication (NFC) and others. The communication links 102 can each use the same or different wireless protocols. The system 100 may be configured to communicate over other wireless data communication links such as, but not limited to, RF communication link, infrared communication link, or any other type of suitable wireless communication connection between two or more electronic devices, which may further be uni-directional or bi-directional communication. Alternatively, the data communication link may include wired cable connection such as, for example, but not limited to, RS232 connection, USB connection, FireWire, Lightning, or serial cable connection.

For example, and as embodied herein, communication link 102 can be configured to use a Bluetooth protocol, such as BLE, or communication link 102 can be configured to use an NFC protocol. Additionally or alternatively, another communication link not shown may exist between a second sensor assembly and it can be configured to use BLE or both NFC and BLE. The communication links can be configured to perform different operations. For example, communication link 102 can be configured to perform only activation of the sensor assembly. Furthermore, communication links can have different configurations depending on the overall system architecture or the components that are activated or being used in the system at a given time. For example, and as embodied herein, communication link 102 can have a first communication configuration when the receiving device 200 is active in the system and a second communication configuration when the receiving device is not active or not included in the system.

In the first communication configuration, the communication link 102 can be configured only to perform activation of the sensor using an NFC wireless protocol. In another configuration, BLE capability (if provided) can remain inactive between the sensor assembly 300 and the applications 420. The application 420 can activate the sensor assembly 300 using NFC wireless protocol and obtain sensor context information. Sensor context information can include authentication information for authenticating a communication session with the sensor assembly 300, encryption information to enable encrypted data communication over the communication links, and a BLE communication address to initiate a BLE connection with the sensor assembly 300. The software library 400 may also obtain the sensor context information from the sensor assembly 300 over BLE. Using the sensor context information, the software library 400 includes capabilities to allow a session to switch from an application 420 on the receiving device 200 such as a smartphone to another application 420 on another receiving device 200 such as a smartwatch. The sensor context information can be transmitted within the applications 420.

In accordance with the disclosed subject matter, the sensor assembly 300 as shown may include sensing elements for detecting different analytes within the same sensor assembly. The system 100 may also include multiple sensor assemblies 300, as shown, connected via a communication link having similar capabilities of communication to the communication link 102 described herein. Two or more sensor assemblies 300 can also be used in conjunction by having multiple sensing elements that together produce the reading for an analyte, or separately produce readings for different analytes. Any number of sensor assemblies could be used together to measure any number of different analyte values, and two sensor assemblies are shown for illustration, not limitation, in this disclosure.

In some embodiments, the application 420 can be configured to access the software library 400 through a remote cloud 700 infrastructure via wireless communication links 710. In certain embodiments, the communication link 710 includes a wireless communication section configured for bi-directional radio frequency (RF) communication with other devices to transmit and/or receive data to and from the system 100. In addition, the communication link 710 may also be configured to include physical ports or interfaces such as one or more of a USB port, an RS-232 port, a serial port, a IEEE 1394 (Firewire) port, an Ethernet port or any other suitable electrical connection port to allow data communication between the system 100 and receiving device 200, such as a personal computer, a laptop computer, a notebook computer, an iPad, a tablet computing device, a cellular telephone, a smart phone, a personal data assistant, a workstation, a server, a mainframe computer, a cloud computing system, an external medical device, such as an infusion device, an analyte monitoring device, or including an insulin delivery device, or other devices that are configured for similar complementary data communication. In certain embodiments, communication link 710 may include a cellular communication protocol, a Wi-Fi (IEEE 802.1x) communication protocol, or an equivalent wireless communication protocol which would allow secure, wireless communication of several units (for example, per HIPPA requirements) while avoiding potential data collision and interference.

In other embodiments, the wireless communication section 710 may be configured for infrared communication, Bluetooth communication, wireless USB communication, ZigBee communication, cellular communication, Wi-Fi (IEEE 802.11 x) communication, RFID (passive or active) communication, or any other suitable wireless communication mechanism to enable the receiving device 200 to communicate with other devices such as infusion devices, analyte monitoring devices, computer terminals, servers, personal computers, laptop computers, notebook computers, iPads, tablet computers, cell phones, smart phones, workstations, mainframe computers, cloud computing systems, communication enabled mobile telephones, personal digital assistants, or any other communication devices with which the patient or user of the device may use in conjunction therewith, in managing the treatment of a health condition, such as diabetes.

The system 100 may be configured to operate as an open loop system, a closed-loop system, and a hybrid closed-loop system. An open loop system requires manual user input to control certain functionalities related to the sensor assembly 300. A closed-loop system uses data from the sensor assembly 300 and algorithms to control the software library 400 without user input. In a hybrid system, input may be required from a user to control the application 420 and initiate the software library 400. A hybrid closed-loop system can be used in conjunction with, or in place of, a closed-loop system. As disclosed herein, regulatory clearance can be limited to software library 400 irrespective of the type of system configuration used in the system 100.

### Receiving Device

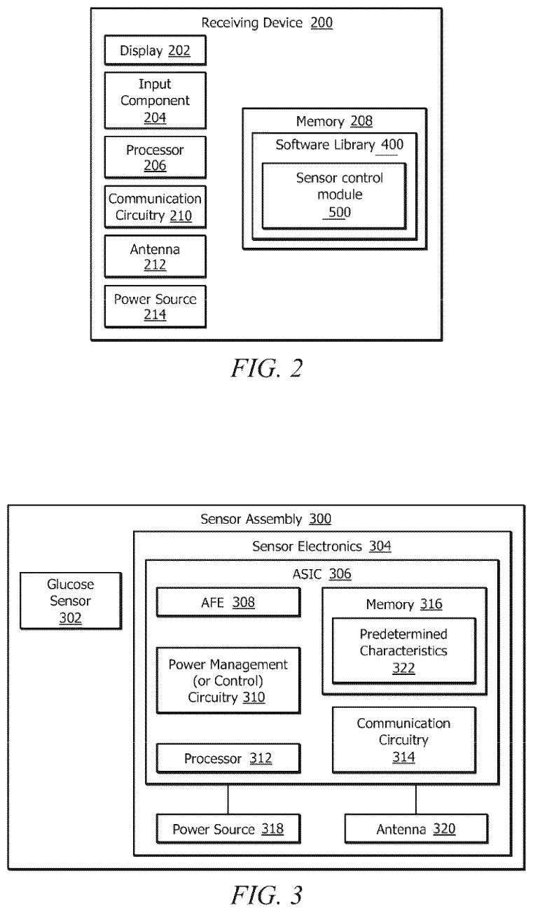

FIG. 2 is a block diagram depicting an example embodiment of a receiving device 200. A software library 400 can be provided to a third-party and incorporated within an application 420 for a multi-purpose receiving device 200, such as a mobile phone, tablet, personal receiving device, or other similar receiving device. Receiving device 200 embodying and executing device application software can also be referred to as a computing device or a multi-purpose device. Receiving device 200 refers to a suitably configured hardware device which is executing an application 420 that incorporates a software library 400 having a sensor control module 500 configured for communication with the sensor assembly 300. Here, receiving device 200 can include a display 202, input component 204, and a processor 206 coupled with memory 208. Also included can be communication circuitry 210 coupled with an antenna 212, and power source 214. As understood by one of skill in the art, these components are electrically and communicatively coupled in a manner to make a functional device. As embodied herein, the memory 208 can include an application and a sensor control module 500 for the sensor assembly 300. The application 420 can also import a software library 400 including the sensor control module 500. The software library 400 and the sensor control module 500 can be developed by the provider of the sensor assembly 300.

The receiving device can have the majority of the processing capability of the system 100 for rendering end-result data suitable for display to a user. The receiving device 200 can be a smartphone or a smartwatch.

The receiving device 200 can receive analyte data, such as glucose data and calculate low and high analyte level and generate corresponding alarms and messages. The receiving device 200 can also mirror an alert generated by another device, such as the sensor assembly 300. The receiving device 200 can process analyte data with the processor 206 and render on the display 202 analyte-related information as value, trend, and graph, and provide additional messaging and notification based on the received analyte level.

### Sensor Assembly

FIG. 3 is a block diagram depicting an example embodiment of a sensor assembly 300 comprising a glucose sensor 302 and sensor electronics 304 (including analyte monitoring circuitry). Glucose sensor 302 can be an in vivo analyte sensor and have a use period of about 13-30 days. Sensor assembly 300 can be without wide-area network communication capability.

The glucose sensor 302 generates raw data signals for measurements of the patient's glucose level. Sensor electronics 304 are operatively coupled to the glucose sensor 302, the sensor electronics 304 comprising a memory 316 storing one or more predetermined characteristics 322 associated with the sensor electronics 304. The memory 316 can be a so-called “one-time programmable” (OTP) memory, which can include supporting architectures or otherwise be configured to define the number times to which a particular address or region of the memory can be written, which can be one time or more than one time up to the defined number of times after which the memory can be marked as unusable or otherwise made unavailable for programming. Subject matter disclosed herein relates to systems and method for updating said OTP memories with new information.

The sensor electronics 304 can include a single semiconductor chip, as depicted, that can be a custom application specific integrated circuit (ASIC 306). Shown within ASIC 306 are certain high-level functional units, including an analog front end (AFE 308), power management (or control) circuitry 310, processor 312, and communication circuitry 314 (which can be implemented as a transmitter, receiver, transceiver, passive circuit, or otherwise according to the communication protocol). As an example, only and not by way of limitation, example communication circuitry 314 can include a Bluetooth Low-Energy (“BLE”) chipset, Near-Field Communication (“NFC”) chipset, or other chipsets for use with similar short-range communication schemes, such as a personal area network according to IEEE 802.15 protocols, IEEE 802.11 protocols, infrared communications according to the Infrared Data Association standards (IrDA), etc. The communication circuitry 314 can transmit and receive data and commands via interaction with similarly capable communication modules. Certain communication chipsets can be embedded in ASIC 306 (e.g., an NFC antennae).

The sensor assembly 300 can use application layer encryption using one or more block ciphers to establish mutual authentication and encryption of other devices in the system 100. The use of a non-standard encryption design implemented in the application layer has several benefits. One benefit of this approach is that in certain embodiments the user can complete the pairing of the sensor assembly 300 and another device with minimal interaction, e.g., using only an NFC scan and without requiring additional input, such as entering a security pin or confirming pairing. Sensor assembly 300 can be configured to dynamically generate authentication and encryption keys. Sensor assembly 300 can also be pre-programmed with a set of valid authentication and encryption keys to use with particular classes of devices. The ASIC 306 can be further configured to perform authentication procedures with other devices (e.g., handshake, mutual authentication, etc.) using received data and apply the generated key to sensitive data prior to transmitting the sensitive data.

In this embodiment, both AFE 308 and processor 312 are used as analyte monitoring circuitry, but in other embodiments either circuit can perform the analyte monitoring function. Processor 312 can include one or more processors, microprocessors, controllers, and/or microcontrollers, each of which can be a discrete chip or distributed amongst (and a portion of) a number of different chips.

Memory 316 included within ASIC 306 and can be shared by the various functional units present within ASIC 306, or can be distributed amongst two or more of them. Memory 316 can also be a separate chip. Memory 316 can be volatile and/or non-volatile memory. In this embodiment, ASIC 306 is coupled with a power source 318, which can be a coin cell battery, or the like. AFE 308 interfaces with glucose sensor 302 and receives measurement data therefrom and outputs the data to processor 312 in digital form. This data can then be provided to communication circuitry 314 for sending, by way of antenna 320, to software library 400.

The glucose sensor 302 can alternatively monitor other analytes, for example, acetyl choline, amylase, bilirubin, cholesterol, chorionic gonadotropin, creatine kinase (e.g., CK-MB), creatine, DNA, fructosamine, glutamine, growth hormones, hormones, ketones, ketone bodies (e.g., ß-hydroxybutyrate), lactate, peroxide, prostate-specific antigen, prothrombin, RNA, thyroid stimulating hormone, and troponin.

The sensor assembly 300 includes a sensor assembly embedded library (not pictured) configured for providing sensor assembly data to the software library 400 based on information received from the sensor assembly 300. Sensor assembly data can include glucose readings, data types, range, real time and historical glucose and trends, sensor operating information, and sensor system information.

### Software Library

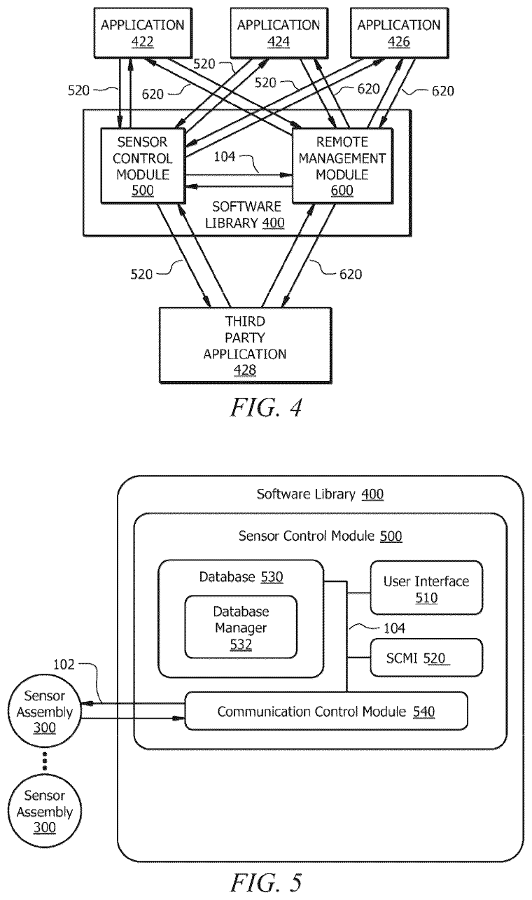

FIG. 4 is a block diagram depicting an example of a software library 400 for communication with applications 420, shown as applications 422, 424, 426, and third-party application 428. References to application 420 refers to one or more of the applications 422, 424, 426 or third-party application 428. Software library 400 includes a sensor control module 500 and a remote management module 600, each of which is capable of independently communicating with applications 422, 424, 426 or third-party application 428. In accordance with the disclosed subject matter, sensor control module 500 and a remote management module 600 may each provide a single uniform interface to communicate with the applications 422, 424, 426 or third-party application 428.

Software library 400 may use a modular architecture and may be made available via a software development kit that can be made for common use by applications 420. The software library 400 may include two modules, each of which could be independently provided for use by other applications 420. The first such module may be a sensor control module 500. The sensor control module may communicate with the sensor assembly 300 and receive a particular result of the value from the sensor assembly 300. The sensor control module 500 may further communicate with applications 422, 424, 426, or third-party application 428 using a sensor control module interface 520.

The software library 400 may further include a remote management module 600 that will be further described below. The remote management module 600 communicates with applications 422, 424, 426, or third-party application 428 using a remote management module interface 620.

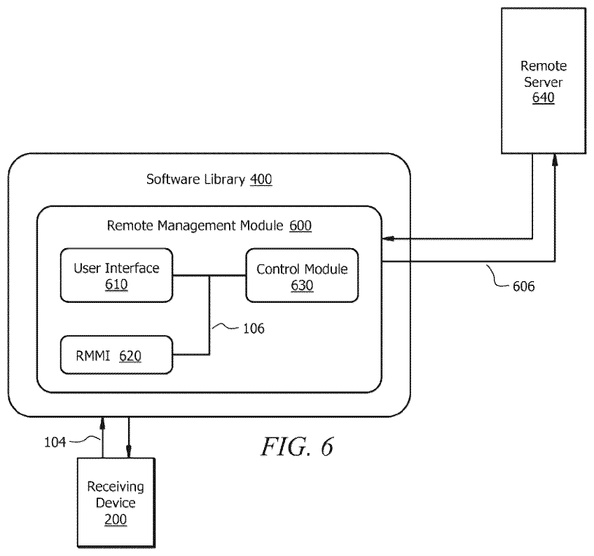

The remote management module 600 may further receive the sensor data from the sensor control module 500 via the inter-module interface 450 and can further be used to store that data in a remote server 640 (shown on FIG. 6) for remote storage, such as in the cloud. By using a remote management module 600, an application developer can also take advantage of a consistent user interface for account management for a user across different third-party applications such as third-party application 428. Data privacy can further be integrated into the remote management module 600 for account management purposes.

The sensor control module 500 may receive a request to initiate the sensor assembly 300. The sensor control module 500 may include logic to identify the particular type of receiving device 200 making the request, and can perform an authentication function for the receiving device 200. Authentication may use a three-pass design with different keys. Keys can be aligned with differential roles (manufacturer, application developer, etc.). Sensitive commands that could leak security information can trigger authenticated encryption using an authenticated additional keyset. The sensor data provided to the sensor control module 500 and sent to the application 422, 424, 426 or third-party application is highly sensitive and can be beneficial to be protected. Medical data associated with a patient is sensitive data at least in part because this information can be used for a variety of purposes, including for health monitoring and medication dosing decisions. As embodied herein, the various modules and applications 422, 424, 426, and third-party application 430 can be configured compliant with a security interface designed to protect the Confidentiality, Integrity and Availability (“CIA”) of this communication and associated data. To address these CIA concerns, to facilitate the confidentiality of data, communication connections between the sensor assembly 300 and the sensor control module 500 can be mutually authenticated prior to transmitting sensitive data. The same would be done for communication between the sensor control module 500 and application 422, 424, 426, and third-party application 428. Communication connections can be encrypted using a device-unique or session-unique encryption key. As embodied herein, the encryption parameters can be configured to change with every data block of the communication.

As embodied herein, to guarantee the integrity of data, encrypted communications between any two components (e.g., a sensor control module 500 and sensor assembly 300) can be verified with transmission integrity checks built into the communications. As embodied herein, session key information, which can be used to encrypt the communication, can be exchanged between two devices after the devices have each been authenticated. Encrypted communications between a sensor assembly 300 and a dedicated sensor control module 500 can be validated with an error detection code or error correction code, including as an example and not by way of limitation, non-secure error-detecting codes, minimum distance coding, repetition codes, parity bits, checksums, cyclic redundancy checks, cryptographic hash functions, error correction codes, and other suitable methods for detecting the presence of an error in a digital message.

The sensor control module 500 may further generate state information to maintain the active status for the receiving device 200 while it remains desirous of the sensor data.

The sensor control module 500 may include a user interface 510 that can enable data sharing for the applications, including necessary permissions to enable data sharing. The user interface 510 at the sensor control module 500 may also display the sensor data received from the sensor assembly 300.

The user interface 510 of the software library is disclosed herein as a modular user interface 510 that allows for sharing and display of the multiple different analytes that can be measured from the different sensor assemblies 300. In particular, as disclosed herein, by using a software library 400 and sensor control module 500, a shared user interface can be developed for display of sensor data from multiple sensor assemblies 300. The user interface 510, when shared, could toggle between sensor data related to the various sensor assemblies 300, display sensor data on one screen, or use multiple different combinations to display the sensor data.

Communication between the sensor control module 500 and applications 422, 424, 426, or third-party application 428 occurs over a sensor control module interface 520. Communication between the remote management module 600 and applications 422, 424, 426, or third-party application 428 occurs over a remote management module 620. The communication is further driven using an event notification or callback process. For example, when the sensor control module 500 receives a request from a third-party application 428 for sensor data, the request may be communicated through the sensor control module interface 520 and an event may be generated at the user interface 510 of the sensor control module 500 to initiate authentication.

As another example, when sensor data is received over communication link 102 by the sensor control module 500, an event can be generated to notify other modules or components within the software architecture that data can be displayed on a user interface 510 of the sensor control module 500.

By having a modular architecture using a software library 400 and sensor control module 500 to interface with the applications 422, 424, 426 and third-party application 428, the system enables communication with different types of sensor assemblies 300, including multiple sensor assemblies 300. In particular, the communication control module 540 may include functionality specific to each of the sensor assemblies 300 within the system, and may simultaneously access and communicate with the various sensor assemblies 300 to receive sensor data.

As another example, a developer of a third-party application 428 may elect to use certain modules of the software library 400 to support the functionalities within the third-party application 428. For example, certain third-party applications 428 may use the sensor data as wellness data. Wellness data can generally include any type of data associated with a person's health, such as their weight, heart rate, blood pressure, blood glucose level, or the like. Sensor assemblies may provide resulting sensor data that may include such wellness data. To the extent a third-party application desires to make use of the sensor data, the third-party application may access the respective module from the software library 400 for the desired sensor data. With the software library 400, the third-party application 428 does not need to directly interface with the sensor assembly 300 to receive sensor data. The software library 400 includes a sensor control module 500 that can receive the sensor data and provide that to the respective third-party application 428. It should be understood that “third party” can correspond to an entity different than the manufacturer of the sensor assembly 300 or software library 400. The third-party application 428 may have access to certain permitted data on database 530 accessible through sensor control module interface 520. Separately, the third-party application 428 may include its own database (not pictured) for storing the sensor data received through the sensor control module 500.

In certain applications, software that operates in conjunction with a medical device such as a sensor assembly sensing data from a user interaction or user health information may be regulated as medical device software. Certain standards pertain to regulation of medical device software, including with reference to ISO 13485:2016 “Medical devices—Quality Management Systems—Requirements for regulatory purposes,” ISO14971:2012 “Medical devices —Application of Risk Management to Medical Devices,” and IEC 62304, Ed 1.1:2015 Medical Device Software—Software Lifecycle Processes.” In particular, regulation requires that software that functions as a medical device (commonly referred to as Software as a Medical Device) is to be regulated by a regulatory agency, such as the Food and Drug Administration in the United States. This regulation at least requires submitting the application for regulatory clearance.

As described by the disclosed subject matter, the regulated portion of software as a medical device can be contained within the software library 400 and the sensor assembly 300. This can allow applications 422, 424, 426, or third-party application 428 not to have to undergo regulatory approval and clearance when making use of the sensor data. In particular, third-party applications may be developed by third-party developers for one or more wellness purposes that will not require the third-party developer to submit the application for approval based on definitions of software as a medical device as the regulated functionalities would all be contained within the software library 400. This will benefit users by allowing the creation of different wellness tracking applications or other uses of the sensor data that may not have originally been considered by the original manufacturer of the sensor assembly 300.

For applications 422, 424, 426, or third-party application 428, a sensor control module interface 520 is used to communicate with the sensor control module 500. By using the sensor control module interface 520, the applications 422, 424, 426 or third-party application 428 can receive data through the sensor control module 500.

The sensor control module 500 may optionally include an alarm module (not pictured) to manage alarms and notifications triggered by the sensor data. In accordance with the disclosed subject matter, the alarm module may include logic to generate alarms for each type of sensor measured by the sensor assembly 300. In particular, the alarms may be triggered if an issue arises with the device hardware of the sensor assembly 300. Additionally, the alarms may be triggered indicating a particular condition with the user being monitored by the sensor assembly 300. In accordance with the modular framework, the alarm logic for the alarm module may be separately maintained within the sensor control module 500.

As described herein, for illustration purposes, the alarm module works with the application 422, 424, 426 or third-party application 428 and the sensor control module 500. The sensor control module 500 receives sensor data from the sensor assembly 300 representing an analyte value. One such value could be a glucose reading. The sensor control module 500 and the alarm module may have threshold detection logic to identify the triggering conditions for an alarm based on a particular analyte value, such as a glucose reading.

During initialization, the third-party application 428 or application 422, 424, 426 can also provide conditions that would require the triggering of an alarm as a callback function. The triggering may involve logic that factors in the value of the sensor data and a temporal relationship. For example, if the sensor assembly provides glucose data, a triggering value may be set to trigger the alarm along with a temporal relationship such as if the value increases by a certain number over a period of time, or remains above a certain value for a period of time. These triggering conditions may also include rate of change as a mechanism to trigger an alarm. By incorporating the alarm module within the sensor control module 500, alarm conditions that require regulatory review and approval can be incorporated within the sensor control module 500, further reducing the need to submit application 422, 424, 426, or third-party application 428 for regulatory approval.

### Sensor Control Module

FIG. 5 is a block diagram depicting an example embodiment of the sensor control module 500 within a software library 400.

In certain embodiments, the sensor control module 500 includes a communication control module 540. The communication control module 540 includes logic to communicate over a communication link 102 to the sensor assembly 300. The communication control module 540 includes further logic for receiving sensor data and displaying the sensor data at a user interface 510. In particular, each sensor assembly 300 includes control logic to perform operations related to sensor communications, especially those that are proprietary. For example, the sensor assembly 300 includes logic provided by the sensor control device's manufacturer to receive sensor measurements and perform complex algorithms on the measurements including data decryption and glucose calculations. In this regard, the communication control module 540 may only need to receive the result of the processing and calculation, with data accuracy and integrity for protection of complex proprietary algorithms occurring at the closed sensor assembly 300. The sensor assembly 300 further includes logic provided by the sensor control device's manufacturer to perform authentication. This allows the sensor assembly 300 to include functionality to provide sensor data that is the resulting data from the sensor measurements for a variety of sensors to the communication control module 540. By using a modular framework, the communication control module 540 includes logic to receive data from a plurality of sensor assemblies 300, enabling substantially simultaneous communication from multiple sensor assemblies 300. This allows authorized third parties to develop mobile apps without requiring that those third parties take on the significant responsibility of independently providing the same level of performance and results accuracy.

This further enables various third-party companies to develop their own mobile applications that work with the manufacturer's sensor assemblies 300 through the software library 400 and the sensor control module 500, with those third-party companies having a variety of use cases that are different from those currently supported by the manufacturer. The utilization of a modular architecture enables third parties to only need to implement a smaller number of interface calls and reference the respective modular components of the software library 400 for implementation.

Communication within the sensor control module 500 to various components occurs over a sensor control module messaging channel 104. The user interface 510 can be used to display the sensor data once received over the sensor control module messaging channel 104.

Applications 422, 424, 426, or third-party application 428 include logic to communicate with the communication control module 540 over a sensor control module interface 520 and operate within the framework to enable receipt of sensor data. The application 420, 424, 426, or third-party application 428 requests sensor control module 500 to perform activation functions by first initiating the sensor control module 500 followed by sending a request to obtain sensor data. The sensor control module 500 includes a sensor control module interface 520 to ensure consistency for overlapping functions required of various applications 422, 424, 426 or third-party application 428. The sensor control module interface 520 is implemented as an application program interface (API) in the underlying application 422, 424, 426, or third-party application 428. A standard interface for shared functions also allows the sensor control module 500 to be used for receipt of sensor data from multiple sensors substantially simultaneously. Logic is contained within the software library for managing the activation of various applications 422, 424, 426 or third-party application 428 that have been authorized to receive sensor data. The sensor control module 500 may further include logic to control and manage the states of the various applications 422, 424, 426 or third-party application 428 via the sensor control module interface 520.

The sensor control module 500 within the software library 400 is positioned as a software as a medical device for regulatory clearance in conjunction with the sensor assembly 300. By housing the components that trigger software as a medical device regulatory issues within the software library communicating with the sensor assembly, additional third-party application 428 avoids the need to be submitted for regulatory approval. This further allows other application developers to build other use cases without having to submit the use-case for the application for regulatory review, and allows unregulated applications to take advantage of the sensor data. This advantage occurs by using the modular logic as described for the software library 400.

The user interface 510 provides a uniform interface for the applications 422, 424, 426 or third-party application 428 to display received sensor data. The user interface 510 may perform a user consent and onboarding function for the applications 422, 424, 426 or third-party application 428. Onboarding includes having a new user of the applications 422, 424, 426 or third-party application 428 completing the necessary consents to have access to the sensor data. The user interface 510 may further include a ready check to determine through the communication control module 540 that the various sensor assemblies 300 are functioning properly. The user interface 510 may include a display functionality to display the sensor data. The user interface 510 can be used in common form for the applications 422, 424, 426 or third-party application 428 for any number of shared functions, such as for account creation of a user, consents for data privacy and sharing, and other similar functions. In accordance with the disclosed embodiments, the sensor control module 500 may present a particular customized user interface 510 when application 422, 424, 426 developed by the manufacturer of the sensor assembly 300 is in operation, but a wholly different user interface 510 for a third-party application 428 that is not developed by the manufacturer of the sensor assembly 300. The look and feel of the user interface 510 thus automatically adjust depending on whether the applications 422, 424, 426 or third-party application 428 has requested the sensor data. As disclosed herein, the sensor control module 500 may be implemented without a user interface 510 component. In this configuration, the sensor control module interface 520 functions to provide information directly to the display of the underlying applications 422, 424, 426 or third-party application 428.

The sensor control module 500 may optionally include integrity and initialization check of accounts to allow connectivity with the sensor and access to the sensor data. Applications 422, 424, 426 or third-party application 428 requests initialization of the sensor control module 500 on start-up of the applications 422, 424, 426 or third-party application 428 by supplying identifying information to the sensor control module 500 and credentials that the sensor control module 500 can use for authentication. If the integrity check fails, the sensor control module 500 will not allow for operation of that application 422, 424, 426 or third-party application 428. For third-party application 428, a remote management module 600 can be used to revoke access to the sensor control module 500 or remove authorization based on the manufacturer's current permissions and goals as determined by the connectivity between the remote management module 600 and the remote server 640. The remote management module 600 can also initiate a process to revoke the authentication of the third-party application 428 from the sensor control module 500 to prevent it from further operation. After successful initialization, the sensor control module 500 initializes the remote management module 600 by providing identifying information and credentials for authentication.

The sensor control module 500 may include protections to ensure that a proper authenticated application 422, 424, 426 or third-party application 428 had made requests for sensor data.

The communication control module 540 may communicate through the communication link 102 to the sensor assembly 300. Using a sensor control module messaging channel 104, the sensor data received from sensor assembly 300 is provided to other components of the sensor control module 500. The sensor data may also communicate to the remote management module 600 via another inter-module interface 450 between the sensor control assembly 500 and the remote management module 600. The sensor data may be further stored in database 530 managed by a database manager 532.

Because of the modular architecture of the software library 400, the communication control module 540 can receive data from any of the various types of sensors represented by sensor assembly 300. This allows for substantially simultaneous receipt of sensor data for the system. Support for multiple different types of sensors occurs at the system level in modular form allowing for future expansion as new sensors are built for tracking additional data by incorporating the necessary modules within the software library 400 and sensor control module 500.

The user interface 510 includes limited functionality to display the sensor data, such as glucose value, and is maintained in this form to allow for uniform use across multiple sensor readings for display of the sensor data. Processing and calculations occur at the sensor assembly 300, and the communication control module 540 receives that sensor data result as a value.

Once the communication control module 540 receives the sensor data, it may post an event by generating an event notification that will inform the respective application 422, 424, 426 that sensor data may be available and accessed through the sensor control module interface 520. The data may be stored in database 530 and accessed directly through the sensor control module interface 520. By using the sensor control module interface 520 and user interface 510, the sensor control module 500 presents a uniform interface for the various applications 422, 424, 426 or a third-party application 428 to activate and receive results of the sensor data. Additionally, the uniform interface 510 includes software logic to identify and register various applications 422, 424, 426 or third-party application 428 to receive certain types of sensor data via callbacks. As an example, if glucose sensor data is available, the uniform interface software logic through sensor control module interface 520 will invoke a callback within the applications 422, 424, 426, or third-party applications 428 authorized to receive glucose sensor data.

The uniform interface logic can use the unique identifier to identify the sensor assembly 300 for which the sensor data request is being made. Although not depicted, according to one aspect of the embodiments, a unique identifier object can be created as an initial step, if one does not already exist. In some embodiments, for example, the unique identifier object can be a user-specific identifier object (e.g., a username, a user profile, or a user account ID) that is inputted, generated, or facilitated by a software application, module, or routine within the software library 400 that is running on the application 420. In other embodiments, the unique identifier object can be associated with a physical device, e.g., a particular sensor assembly 300, and can comprise, for example, a serial number, a media access control (MAC) address, a public key, a private key, or a similar string of characters.

According to another aspect of the embodiments, each of the applications 422, 424, 426, or third-party application 428 includes parameters that can be passed to the sensor control module 500 when a respective call is made by an application 422, 424, 426, or third-party application 42. These various structures and data types can be made available to the sensor control module 500 to assist the sensor control module 500 in accessing the sensor assembly 300 to receive sensor data.

According to another aspect of the embodiments, the sensor control module 500 may store the meta data and state information associated with the sensor assembly 300 or application 422, 424, 426 or a third-party application 428. The sensor control module 500 may further store this data in encrypted form, such as by using the identifier related to the receiving device 200 or sensor assembly 300, state information, and any other information that is useful for establishing and maintaining a connection with the sensor assembly 300, application 422, 424, 426 or a third-party application 428. This database may be separate from the database accessible by the application 422, 424, 426 or a third-party application 428, despite being an active component (though generally inaccessible) component within the application 422, 424, 426 or a third-party application 428. An application 422, 424, 426 or third-party application 428 can also be deactivated or have its access removed from the sensor data.

The sensor control module 500 as embodied herein can identify the application 422, 424, 426 or third-party application 428 based on tag information. When a particular application 422, 424, 426 or third-party application 428 requests access to the sensor data, the sensor control module 500 may identify the application because the sensor control module 500 may be pre-loaded with tagging information corresponding to the application 422, 424, 426 or third-party application 428.

The current framework and system may be compatible with prior applications developed by the manufacturer of the sensor assembly 300. In particular, logic for converting sensor readings into usable data may be included within the sensor assembly 300 or within the respective application 422, 424, 426. In this manner, the system may take advantage of the framework to integrate prior developed applications into the framework of the system.

The sensor control module 500 also has logic to identify whether the request for sensor data comes from an application 422, 424, 426 or a third-party application 428. The sensor control module may further communicate information regarding a sensor data request to a remote management module 600.

The sensor control module 500 may also have logic to receive information regarding hardware issues with the sensor components of the sensor assembly 300. The sensor control module 500 may send a communication to the application 422, 424, 426 or a third-party application 428 to display a status message about an issue with the sensor assembly 300, such as by alerting the user through the application 422, 424, 426 or a third-party application 428 that a sensor is expiring, has a hardware malfunction, or some other problem that would interfere with providing sensor data related to the analyte being monitored by the sensor assembly 300. The sensor control module 500 may send a communication to the receiving device 200 operating system when the application 422, 424, 426 or third-party application 428 is in the background to display a notification identifying an issue with the sensor assembly 300. These issues may include that a sensor is expiring, has a hardware malfunction, or some other issue that would interfere with providing sensor data relating to the analyte being monitored by the respective sensor assembly 300.

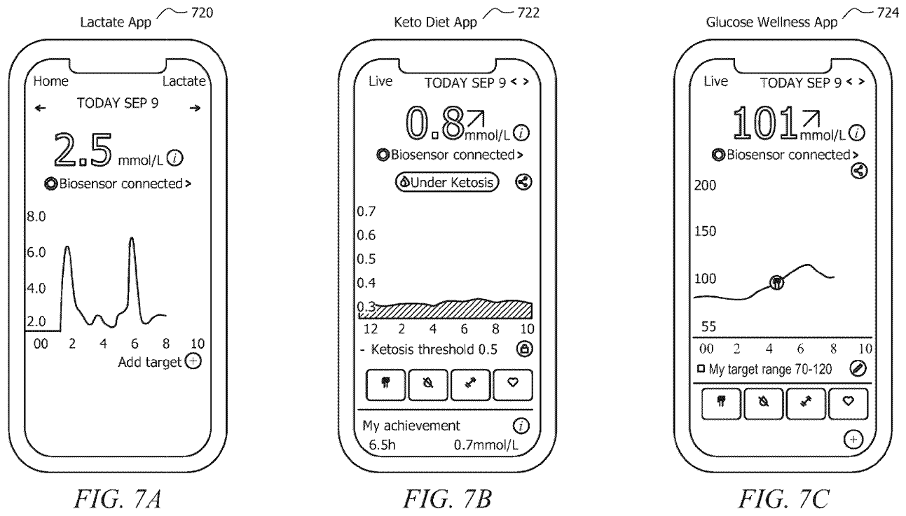

The application 422, 424, 426 or a third-party application 428 may include a user interface (shown further at FIGS. 7A-7C below), including a touch or voice command input, that acts as an interface to receive commands from a user. These commands or input may include a user requesting a sensor reading, visually tapping a display to get sensor data, acknowledging an alarm, or any number of different operations that could be conducted on the display of sensor data.

The sensor control module 500 may be coded in a modular fashion that allows for upgrading the software library 400 to add functionality to communicate with newly developed sensor assemblies. Variables are used in place of hard coded values to enable for modification of the sensor control module 500 to enable communication with newly developed sensor assemblies and to allow applications 422, 424, 426 or a third-party application 428 to get sensor data from those newly developed sensor assemblies without having to submit the underlying application in a new submission or an amended filing for regulatory review and clearance.

### Remote Management Module

FIG. 6 is a block diagram depicting an example embodiment of the remote management module 600.

The user interface 610 of the remote management module 600 provides functionality for applications 422, 424, 426 or a third-party application 428 to have a consistent interface for certain shared functions. As embodied herein, these features and functions can include activities such as data privacy, user consent, third-party consent, application authorization, and more. The user interface 610 of the remote management module 600 provides a consistent interface to allow various applications 422, 424, 426 or a third-party application 428 access to these functions. Communication within the remote management module 600 to various software logic can occur using the remote management module messaging channel 106. The user interface 610 also allows for consistent account management capabilities, allowing a user to create an account, set a password, or set profile related information.

The remote management module 600 further includes a remote control module 630 that enables communication to a remote server 640. The communication with the remote server 640 may occur wirelessly using any available communication means, including BLE and NFC communication.

In an embodiment of the system, the remote management module 600 may further provide transport capabilities for enabling a backup of data stored in the various applications 422, 424, 426 or a third-party application 428 in the event a user upgrades the smartphone or receiving device 200. The remote management module 600 may also communicate with the applications 422, 424, 426, or third-party application 428 over a remote management module interface 620.

The software library 400 including the sensor control module 500 and remote management module 600 may include secure coding layers to assist in the prevention of cyber threats, such hacking and remote access. In one example, protection against such threats may include the use of digital certificates or profile provisioning.

A sensor control module 500 can further identify whether the request for sensor data is generated by an application 422, 424, 426 or a third-party application 428. The sensor control module as embodied herein may pass that information to a remote management module 600 through inter-module interface 450, and the remote management module 600 can further customize the user interface 610 for that application 422, 424, 426 or a third-party application 428 using the remote infrastructure. As part of the customized user interface, a custom user authentication interface may be presented to a user of the application 422, 424, 426 or a third-party application 428. The remote management module 600 further includes logic to disable authentication for application 422, 424, 426 or a third-party application 428. In particular, allowing the remote management module 600 to disable access by a third-party application 428 by removing authorization for the third-party application 428 improves monitoring and control over the applications 422, 424, 426 or third-party application 428 that access sensor data.

### Set-Up

After initiation of set-up of the biosensor, a series of GUIs may be presented to assist the user in applying the biosensor to their skin surface and to pair the biosensor with the application. In some embodiments, the set-up GUIs may only be initiated by selecting the banner. In other embodiments, the set-up GUIs may be opened thorough a set-up button or settings link or button.

The application may present numerous GUIs that describe how to apply the biosensor. A GUI may be presented that shows what is included in the box. In addition to a picture, it may also explain that the user may find a biosensor pack and a biosensor applicator in the box. Another GUI may appear that instructs the user to choose a site on the back of their upper arm away from scars, moles, stretch marks, or lumps, and may be accompanied by a picture highlighting a section of a person's upper arm where it would be appropriate to apply the biosensor. An additional GUI may be provided that instructs the user to clean the selected site by washing the site using plain soap, cleaning the site with an alcohol wipe, and then allowing the site to dry.

The application may provide a GUI that explains how to prepare the biosensor pack and applicator. In addition to a picture showing how to open the pack, the GUI may contain written instructions to peel the lid completely off the biosensor pack and to unscrew the cap from the biosensor applicator. The application may then provide a GUI that includes a picture of a person loading the biosensor in the applicator along with instructions that state to line up the dark marks on the biosensor applicator and the biosensor pack. On a flat, hard surface, press down firmly on the biosensor applicator until it comes to a stop. The application may then provide another GUI that instructs the user to lift the biosensor applicator out of the biosensor pack and informs the user that the biosensor applicator is ready to apply the biosensor. The GUI may include a warning that the biosensor applicator now contains a needle and that the user should not touch inside the biosensor applicator or put it back into the biosensor pack.

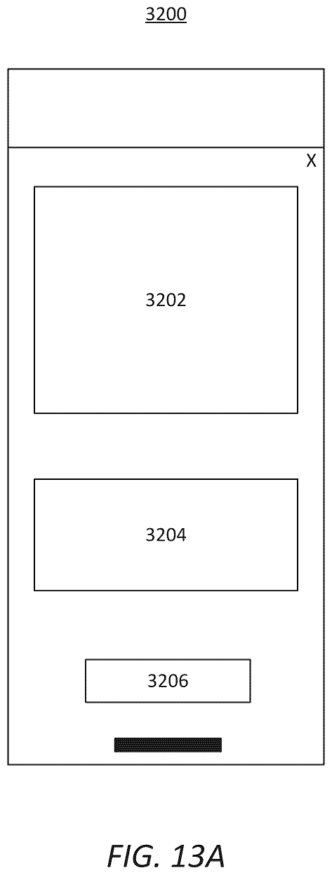

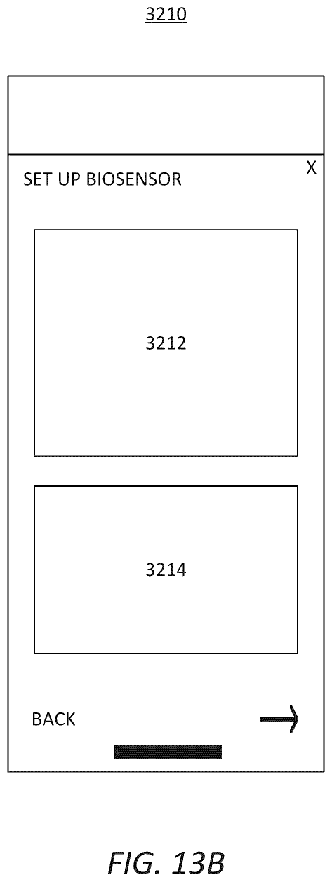

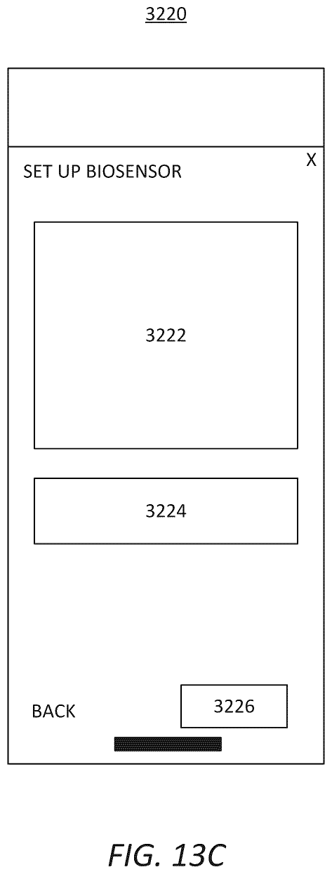

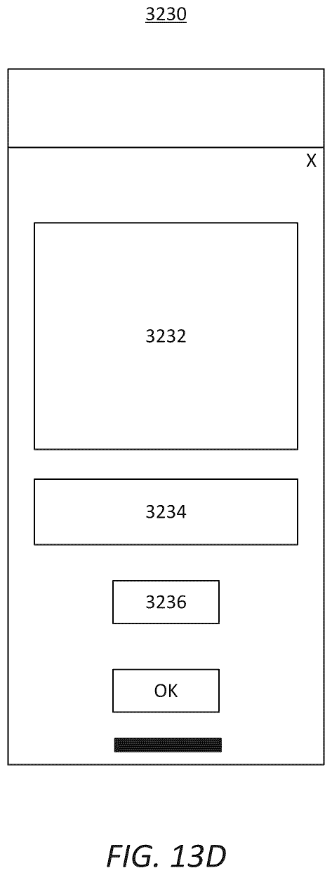

The application may then provide GUIs describing how to apply the biosensor to the user's body. As seen in GUI 3200 in FIG. 13A, an introductory GUI 3200 may include a graphic 3202 of a biosensor, along with introductory text 3204 inviting the user to begin the setup to pair their biosensor. The user may then tap or select a start setup button 3206 to begin the setup process.

As seen in GUI 3210 of FIG. 13B, a Set Up Biosensor GUI 3210 may include a graphic or picture 3212 showing a person applying an applicator to their body at, e.g., the back of their upper arm. The GUI 3210 may also include text 3214 explaining how to apply the biosensor. The text 3214 may instruct the user to place the biosensor applicator over a site and push down firmly to apply the biosensor. The text 3214 may then instruct the user to gently pull the biosensor applicator away from their body. The text 324 may also caution the user not to push down on the biosensor applicator until it is placed over the prepared site to prevent unintended results or injury. The user may then tap the arrow button to proceed to GUI 3220 after the biosensor has been applied.

The application may also present a GUI instructing the user to check the biosensor to make sure that the biosensor is secure by pressing down on the adhesive.

As seen in GUI 3220 of FIG. 13C, in another Set Up Biosensor GUI 3220, a graphic or picture 3222 may show a person pairing the biosensor to the reader device, such as a smart phone, by holding the reader device in close proximity to the applied biosensor. The text 3224 may instruct the user to pair their biosensor by tapping the start pairing button 3226. A pop-up window may appear that instructs the user to hold the reader device (e.g., mobile phone) very close to the biosensor. The phone may vibrate after successfully scanning the biosensor.

After the biosensor has been paired, as seen in FIG. 13D, GUI 3230 may be displayed, which indicates the time remaining for the biosensor to be ready. The GUI 3230 may include a graphic 3232 highlighting the time remaining until the biosensor is active. The graphic 3232 may include a radiating circle of dots, which may be animated. Alternatively, the graphic 3232 may include a progress indicator that may be a bar having a colored portion or may be a colored portion along the perimeter of a circle, where the colored portion is proportional to the amount of time remaining before the sensor is active, e.g., a total perimeter of a circle may be equivalent to 60 minutes and a colored portion of the perimeter may be proportional to the amount of time remaining under an hour for the biosensor to be active. Alternatively, the graphic 3232 may be animated and the color of the perimeter of the circle may change as the time remaining is counting down. The GUI 3230 may also include a display 3236 of the number of minutes until the biosensor is active (e.g., “55:10” where 55 minutes and 10 seconds remain until the sensor is ready or active). Alternatively, in some embodiments, a message of “Ready in 55 mins” may be displayed on the inside of a circle or below the graphic 3236, where a colored portion of the perimeter of the circle is animated and cyclically changes color as the time remaining until the biosensor is active counts down. The GUI may also include a plurality of selectable links that, when selected by the user, can display additional information to the user as explained elsewhere, including how to replace a biosensor, support, learning more about the application, and ordering a biosensor.

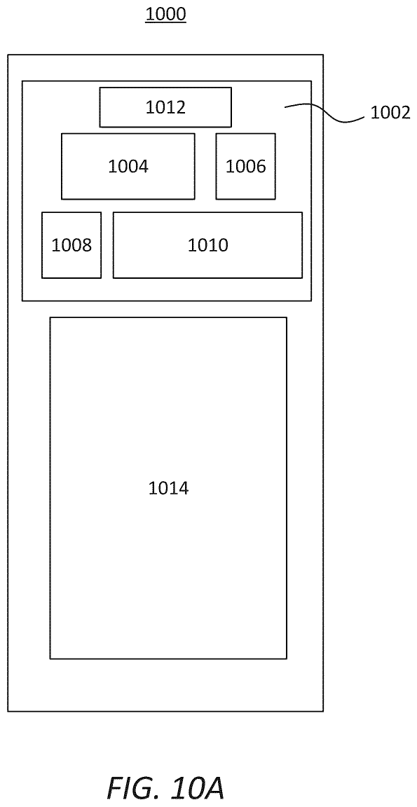

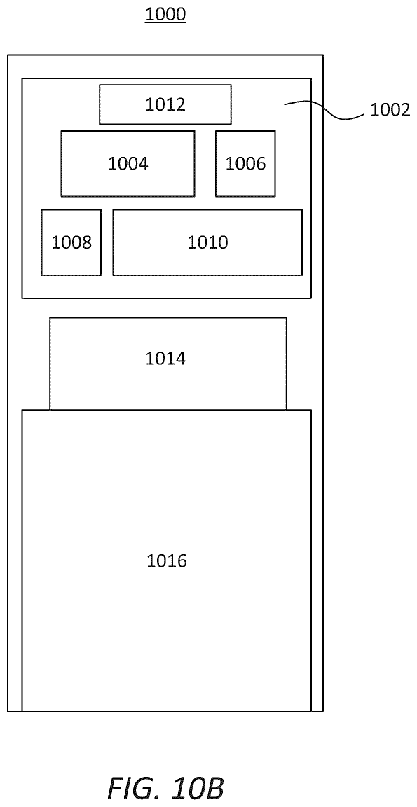

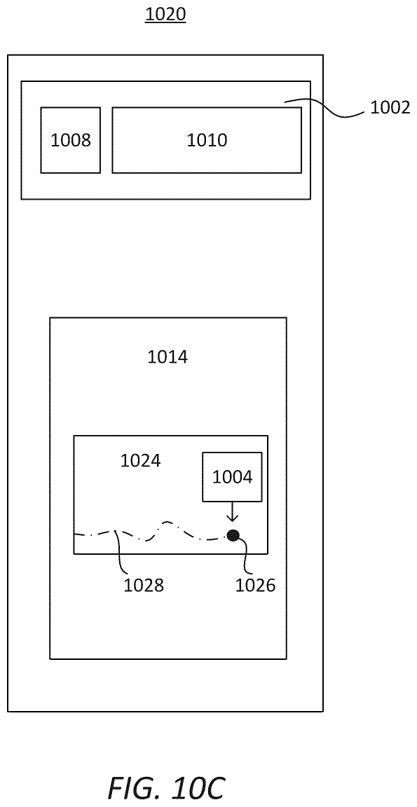

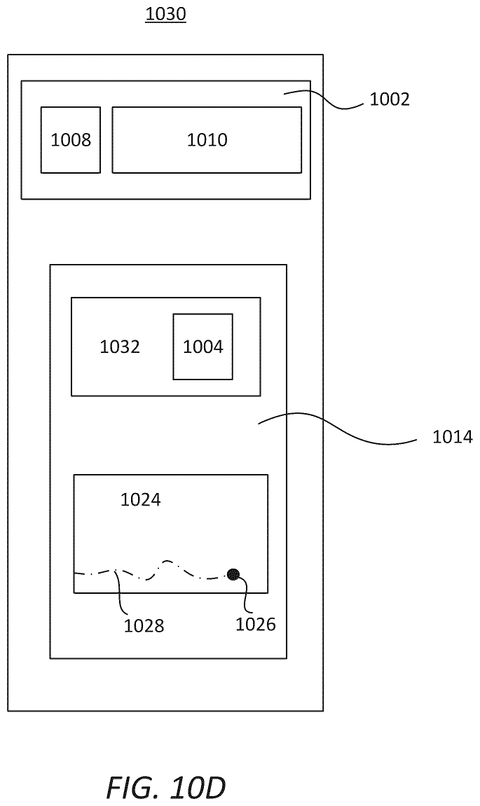

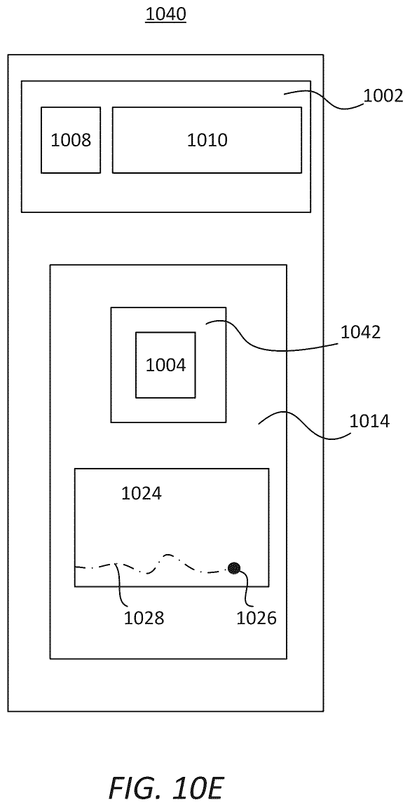

The GUI 3230 may also include a description or message 3234 that informs the user that their biosensor is now getting to know them, and real-time analyte levels will be available in the amount of time displayed in 3236. In some embodiments, elements of GUI 3230 may be provided by the biosensor module. For example, the graphic having a progress indicator 3232 and the display 3236 of the amount of time until the biosensor is active may be provided by the biosensor module. After the user clicks OK, the GUI 3230 may collapse and the home screen with the banner may then be visible to the user. As seen in FIG. 10A, the banner 1002 may display an icon 1008 indicating the status of the biosensor along with information 1010 regarding the status of the biosensor. In some embodiments, similar to the progress indicator described with respect to graphic 3232, the icon 1008 may also include a progress indicator that indicates the time remaining for the biosensor to be ready. The icon 1008 may include a graphic highlighting the time remaining until the biosensor is active. The graphic may include a radiating circle of dots, which may be animated. The icon 1008 may include a progress indicator that may be a bar having a colored portion or may be a colored portion along the perimeter of a circle, where the colored portion is proportional to the amount of time remaining before the sensor is active, e.g., a total perimeter of a circle may be equivalent to 60 minutes and a colored portion of the perimeter may be proportional to the amount of time remaining under an hour for the biosensor to be active. The information 1010 may include text indicating that the biosensor is “READY IN XX,” wherein XX may be displayed in minutes and seconds. For example, the banner 1002 may display “READY IN 55:10” where the biosensor will be active in 55 minutes and 10 seconds.

### Applications

FIG. 7A-7C are exemplary embodiments of applications using the software library 400 and sensor control module 500.

In one example, application 420 may be an application to track analyte values such as lactate as shown in FIG. 7A, ketones or ketone bodies (e.g., ß-hydroxybutyrate), such as shown in FIG. 7B, or glucose, as shown in FIG. 7C. Part of the display may come from the sensor control module interface 520 and part may be displayed based on processing within the underlying application 420.

Furthermore, according to some embodiments, applications 720, 722, 724 represent applications 422, 424, 426 to communicate with the sensor control module 500 to enable receipt of sensor data. By using the sensor control module 500 and remote management module 600, a consistent user experience can be provided for the different applications. Moreover, if an additional analyte value needs to be detected and sensed, that application can further integrate the updated software library 400 without having to develop the full architecture for communication, account management, user privacy, and consents.

The improvements to the GUI in the various aspects described and claimed herein produce a technical effect at least in that they assist the user of the device to operate the device more accurately, more efficiently and more safely. It will be appreciated that the information that is provided to the user on the GUI, the order in which that information is provided and the clarity with which that information is structured can have a significant effect on the way the user interacts with the system and the way the system operates. The GUI therefore guides the user in the technical task of operating the system to take the necessary readings and/or obtain information accurately and efficiently.

### Biosensor Module Banner

As referenced above, user interface 510 of the sensor control module 500 may include various components. As seen in FIG. 10A, GUI 1000 may include a banner 1002, which is created by the user interface 510, that may be incorporated into GUIs generated by the host application, e.g., applications 422, 424, 426 or third-party application 428. The banner 1002 generated by the user interface 510 may show a different interface depending on the host application into which it is integrated. The banner 1002 may include a real time concentration value 1004, a trend arrow 1006, an icon 1008 indicating the status of the biosensor, information 1010 regarding the status of the biosensor, and an additional indication of the biosensor status 1012. The banner 1002, or elements within the banner 1002, may be selectable to link to other GUIs with additional information about the biosensor. For example, where the host application is a glucose wellness application, the banner 1002 may include a real time concentration value 1004, a status icon 1008, and information status 1010 about the biosensor. Moreover, the real time concentration value 1004 may be located in a different part of the GUI (e.g., as part of an analyte graph) than the rest of the banner.

Various different status icons 1008 and information regarding the status of the biosensor 1010 may be displayed. In some embodiments, a status icon 1008 including a circle of dots, which may or may not be animated, may appear next to status information 1010 indicating that the biosensor is not connected or ready. For example, the status information 1010 may state that the biosensor will be ready in a certain amount of time, e.g., “Ready in 30 mins.” In some embodiments, the status information 1010 may state “Searching,” which may indicate that the application is trying to connect to the biosensor. In some embodiments, the status icon may be a circle or an annular ring. If the biosensor is connecting, the ring may have a black dot located at a point along the perimeter of the circle. If there is an error with the biosensor, or if the biosensor is ending soon, then the status icon may have a red dot located at a point along the perimeter. The status icon may be animated such that the dot travels around the perimeter of the circle or annular ring.

In some embodiments, as seen in FIG. 10B, a pop-up screen 1016 may also appear over GUI 1000. The pop-up screen 1016 may convey a message regarding the biosensor starting up. The pop-up screen 1016 may indicate the amount of time in, e.g., hours and/or minutes, remaining until the biosensor is ready. For example, the pop-up screen may indicate that the “Biosensor is ready in 55 minutes.” In other embodiments, if the reader device, such as a smart phone, is locked, a notification may appear on the lock-screen indicating that the biosensor is ready.

As seen in FIG. 10A, in some embodiments, a status icon 1008 including a circle with a color progress indicator may appear next to status information 1010 stating that the biosensor is connected and working properly. For example, the status information 1010 may say “LIVE.” In some embodiments, for the status icon 1008 of the circle with the progress indicator, the progress indicator may be colored, and the color may be proportional to the amount of sensor life remaining for the current biosensor. In some embodiments, the color of the progress indicator may be different colors depending on how much sensor life is remaining. For example, the color indicator may be a blue color if at least about 50%, alternatively at least about 40%, alternatively at least about 30%, alternatively at least about 25%, alternatively at least about 20%, alternatively at least about 10% of the sensor life remains. In some embodiments, the colored progress indicator may be a different color, e.g., orange or red, if the sensor life remaining is less than a certain amount. For example, the color indicator may be an orange color if less than about 50%, alternatively less than about 40%, alternatively less than about 30%, alternatively less than about 20%, alternatively less than about 10%, alternatively less than about 5% of the sensor life remaining.

In some embodiments, the status information 1010 may state “SEE DETAILS” or similar language to indicate that the user should learn more about the status of the biosensor. By selecting on the “SEE DETAILS” or other parts of the banner, one of many explanations, may be displayed to indicate a possible error or issue with the biosensor. In some embodiments, where there is an issue with the biosensor and the status information 1010 states “SEE DETAILS,” a real time concentration value 1004 may not be displayed. In lieu of the real time concentration value 1004, in some embodiments, a plurality of dashes or dots (e.g., two dashes) may appear instead of the concentration value 1004 of the analyte measured by the biosensor.

In some embodiments, the banner 1002 may include a real time concentration value 1004, an icon 1008 indicating the status of the biosensor, and information 1010 regarding the status of the biosensor. As seen in FIG. 10C, a GUI 1020 may have the real time analyte concentration value 1004 located in a different part of the GUI than the other components of the banner 1002. For example, the real time analyte concentration value 1004 may be located in a graph 1024 that is part 1014 of the GUI generated by the host application. The graph may include analyte curve 1028 and a marker 1026 for the current analyte concentration, and the real time analyte concentration value 1004 may be located above the marker 1026. In other embodiments, as seen in FIG. 10D, the real time analyte concentration value 1004 in GUI 1030 may be located in a sentence or statement 1032 about the user's analyte concentration. For instance, the sentence or statement 1032 may be located above the graph 1024 and may say, for example, “Your glucose is 124 mg/dL. Your number is steady, and you're doing great!”

In some embodiments, the banner 1002 may include a real time concentration value 1004, an icon 1008 indicating the status of the biosensor, and information 1010 regarding the status of the biosensor. As seen in FIG. 10E, the real time analyte concentration value 1004 in GUI 1040 may be located in a graphic element 1042 provided by the host application. The graphic element 1042 may be a colored circle or other shape. If the analyte level is within a target range or determined to be steady, as explained elsewhere in this application, the graphic element 1042 may be colored a first color (e.g., green). If the analyte level is outside of a target range, determined to be unsteady (as explained elsewhere in this application), or a spike in the analyte concentration is detected, the graphic element 1042 may be colored a second color (e.g., orange). The color of the graphic element may be the same color as the analyte curve 1028 in the analyte graph 1024.

### System Messages

If there is an issue with the biosensor, a system message may appear regarding the status of the biosensor. The system message may appear in a pop-up window or alert. Alternatively, in some embodiments, the system message may appear after the user selects “SEE DETAILS.”

In some embodiments, the details message 1210 may include a “Pairing Error” message that may state that the pairing was unsuccessful. Moreover, the application may suggest trying to pair the biosensor again.

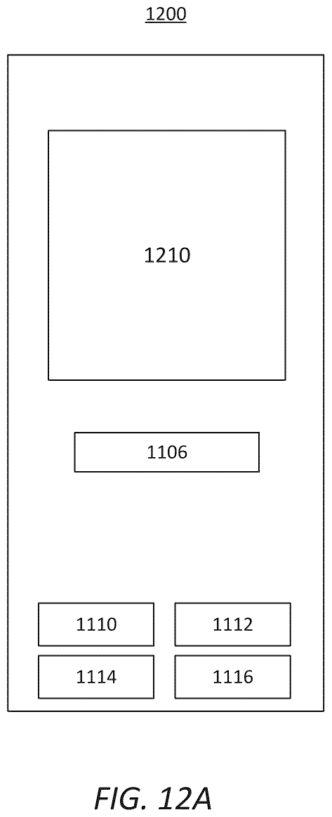

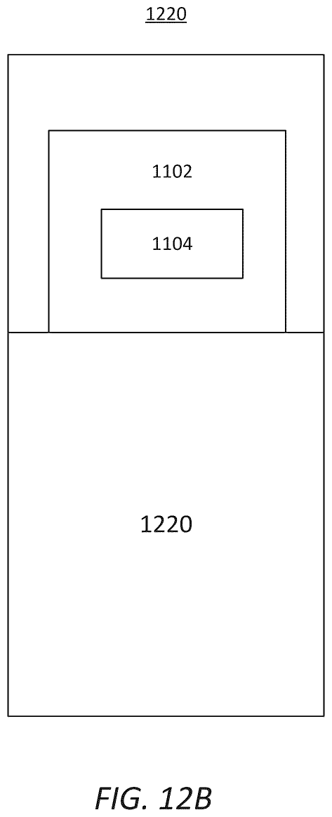

After a user selects “SEE DETAILS,” as seen in FIG. 12A, user interface 510 of the sensor control module 500 can be configured to display one of a plurality of messages in a GUI 1200 that may include a details message 1210, the serial number of the current biosensor 1106, and a plurality of selectable links 1110, 1112, 1114, 1116 that, when selected by the user, can display additional information to the user as explained elsewhere. In some embodiments, the details message 1210 may be presented in a circular graphic that includes a progress indicator to visually illustrate the remaining life of the current biosensor. As explained with respect to other embodiments, the graphic may be a circle and the progress indicator may be a different color perimeter of the circle, where the progress indicator may be proportional to the amount of sensor life remaining for the current biosensor. And as explained with respect to other embodiments, the color of the progress indicator may be different colors depending on how much sensor life is remaining.

In some embodiments, the banner 1002 may display a plurality of dashes or dots (e.g., two dashes) instead of the concentration value or level 1004 of the analyte measured by the biosensor when there is an issue with the biosensor. If the user taps on the banner 1002 when the banner is not displaying a real-time analyte concentration or level, e.g., displaying plurality of dashes or dots, then one of a plurality of system messages regarding problems with the biosensor may be displayed. When the user taps or selects the banner, a details message GUI may appear that gives additional details about the biosensor status.

In some embodiments, the details message 1210 may include a “Check Biosensor” message that may state that it looks like the user's biosensor isn't applied correctly. The details message 1210 may also include further instructions indicating if the biosensor is not attached firmly to the user's skin, then the user should apply a new biosensor and pair it. The details message 1210 may also include further instructions indicating if the biosensor is applied properly, then the user should try pairing the biosensor again. The details message 1210 may optionally also include a selectable “Pair” or “Pair Biosensor” button that, when selected, would display a GUI that assists the user to begin the pairing process.

In some embodiments, the details message 1210 may include a “Signal Loss” message that may caution the user to keep their phone in range of the biosensor at all times. The details message 1210 may further instruct that if the user continues having issues, then they should turn their phone's Bluetooth off and on, or restart their phone.

In some embodiments, the details message may relate to the temperature of the biosensor. In some embodiments, the details message 1210 may include a “Biosensor Too Hot” message that may inform the user that the biosensor is too hot to give readings. The details message 1210 may further request that the user to please check again in a few minutes. In some embodiments, the details message 1210 may include a “Biosensor Too Cold” message that may inform the user that the biosensor is too cold to give readings. The details message 1210 may further request that the user to please check again in a few minutes.

In some embodiments, the details message 1210 may include a “Biosensor Error” message that may inform the user that a biosensor reading is unavailable. The details message 1210 may further request that the user to please check again in a certain time period, e.g., 5 minutes, alternatively 10 minutes, alternatively 30 minutes.

In some embodiments, when the signal from the biosensor is suddenly lost, the biosensor status may change immediately to “SEARCHING”. When the “searching” description is selected or tapped, a details message 1210 may appear that suggests that the user keep their phone in range of the biosensor at all times. If the user continues having issues, the application suggests that the user turn the phone's BLUETOOTH® off and on, or restart the phone.

In some embodiments, the details message 1210 may include a “Biosensor Incompatible” message that may inform the user that the biosensor cannot be used with this version of the application. The message may suggest removing the biosensor and pairing a new one.

In some embodiments, the details message 1210 may include a “Biosensor Ended” message that may inform the user that the biosensor has ended and instruct the user to pair a new biosensor.

In some embodiments, the details message 1210 may include a “Biosensor already in use” message that may inform the user that a biosensor has already been paired and cannot be used. The message may also instruct the user to remove the biosensor and pair a new one.

In some embodiments, the details message 1210 may include a “Current Biosensor” message, which may be in a different color than messages indicating problems or errors. The message may indicate that the biosensor that the user tried to pair is in use and that the user will automatically receive real-time analyte readings directly to their device. The message may also include a different icon, such as a check mark.

In some embodiments, the details message 1210 may include an “ENABLE BLUETOOTH” message that requests the user to please turn on BLUETOOTH. The details message 1210 may further explain that BLUETOOTH is required to receive biosensor readings.

In some embodiments, the details message 1210 may include a “Replace Biosensor” message that may inform the user that the biosensor is not working. The details message 1210 may further request that the user please remove the biosensor and pair a new one.

As seen in FIG. 12B, a pop-up window 1220 may also appear, which may include a details message. The contents of the details message in pop-up window 1220 may be the same or substantially the same as described with respect to the plurality of details message 1210 described above that may appear in GUI 1200.

The biosensor module may also create and store an error log.

### Biosensor Module Details GUI

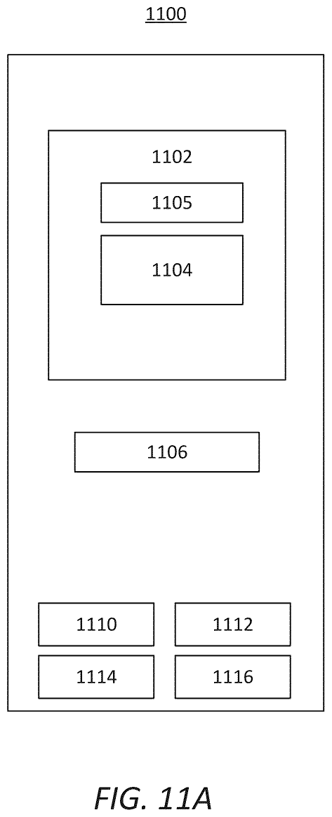

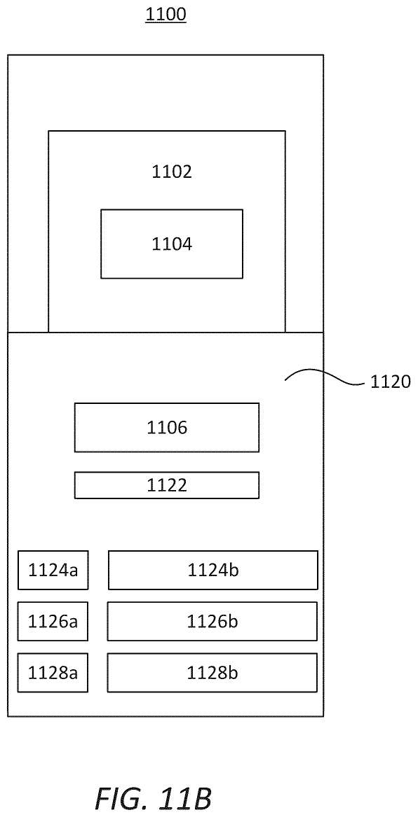

If the user selects any of the elements of the banner 1002, user interface 510 of sensor control module 500 can be configured to output a biosensor module details GUI 1100, as seen in FIG. 11A. The biosensor module details GUI 1100 may include a graphic 1102 that includes a progress indicator to visually illustrate the remaining life of the current biosensor. In some embodiments, the graphic 1102 may be a circle and the progress indicator may be a different color perimeter of the circle, where the progress indicator may be proportional to the amount of sensor life remaining for the current biosensor. For example, for a biosensor with a total life of X days, where the biosensor has Y days of life remaining, then the color indicator around the circumference will be a different color, e.g., blue, for Y/X*100 of the circumference of the circular graphic 1102. For example, for a biosensor with a total life of 14 days, where the biosensor has 12 days of life remaining, then the color indicator around the circumference will be blue for approx. 85.7% of the circumference of the circle 1102.

In some embodiments, the color of the progress indicator may be different colors depending on how much sensor life is remaining. For example, the color indicator may be a blue color if at least about 50%, alternatively at least about 40%, alternatively at least about 30%, alternatively at least about 25%, alternatively at least about 20%, alternatively at least about 10% of the sensor life remains. In some embodiments, the colored progress indicator may be a different color, e.g., orange or red, if the sensor life remaining is less than a certain amount. For example, the color indicator may be an orange color if less than about 50%, alternatively less than about 40%, alternatively less than about 30%, alternatively less than about 20%, alternatively less than about 10%, alternatively less than about 5% of the sensor life remaining.

The GUI 1100 may also include a real time indicator of amount of time remaining of the biosensor life 1104. For instance, the real time indicator may state “12 days” in the middle of the circular graphic 1102, in the example above. The real time indicator 1104 may be proportional to the progress indicator of the graphic 1102. The real time indicator 1104 may indicate the amount of time remaining of the biosensor life as a number of days when the amount of time remaining is greater than about 1 day, alternatively greater than about 23 hours and 59 minutes. The real time indicator 1104 may indicate the amount of time remaining as a number of hours when the amount of time remaining of the biosensor life is less than about 24 hours. In some embodiments, when the amount of time remaining of the biosensor life is less than about 24 hours, then the progress indicator may switch to a different color, e.g., the color may change from blue or green or orange or red. The real time indicator 1104 may indicate the amount of time remaining as a number of minutes when the amount of time remaining of the biosensor life is less than about 1 hour or less than about 61 minutes. The GUI may also include an additional message 1105 stating that the Biosensor Life is “Ending Soon.”

The GUI 1100 may also list the serial number of the current biosensor 1106, which may assist the user if seeking assistance with customer service. If the user selects the serial number 1106, as seen in FIG. 11B, a pop-up screen 1120 with additional details about the biosensor may appear. The pop-up screen 1120 may include the serial number of the current biosensor 1106 and the status of the current biosensor 1122. The pop-up screen 1120 may also include a list of past biosensors 1124, 1126, 1128. The list of past biosensors may include a date 1124a, 1126a, 1128a associated with each biosensor (e.g., the date of activation, or the date that the biosensor was disconnected), along with the serial number and status of the past biosensor 1124b, 1126b, 1128b. In some embodiments, the serial numbers of the current and past biosensors and other information may be copied to assist the user in conveying this information if needed to, e.g., their HCP or customer support.

The GUI 1100 may also include a plurality of selectable links 1110, 1112, 1114, 1116 that, when selected by the user, can display additional information about how to apply the biosensor, options to buy additional biosensors, instructions for use, how to replace a biosensor, support, learning more about the application, learning more about the biosensor, etc.

### Support

If the user taps or selects the link to “Support,” a GUI may include a Help and Learning section, an ABOUT section, and a Customer Care section, which includes details about how to contact a help line. The Help and Learning section may include links to FAQs, Understanding Glucose Readings, and App Tutorial. The ABOUT section may include links to Biosensor IDs, Error History, and App and Biosensor information. As explained elsewhere, the Biosensor IDs link may display a GUI that includes the serial number of the current biosensor, along with a list of past biosensors. The list of past biosensors may include a date associated with each biosensor (e.g., the date of activation, or the date that the biosensor was disconnected), along with the serial number and status of the past biosensor. The Error History link may display a GUI listing each error code, a brief description, and a time and date that the error occurred. The App and Biosensor information link may display a GUI listing the name of the application, the software full version, the SDK version, the OS version, the smartphone model, the country, and a reference number.

### Notifications

In some embodiments, the biosensor module may provide in-app notifications to alert the user of the status of the biosensor. The in-app notifications may also alert the user as to possible actions to take related to the biosensor. The biosensor module may function to facilitate the host application to display operating system-based notifications related to the status of the biosensor (e.g., informing the user that the biosensor has ended). The in-app notifications may appear in a pop-up notification with the host application in the foreground. In some embodiments, the in-app notifications provided by the third-party application related to the status of the biosensor may only be provided by the biosensor module.

The notifications may relate to the status of the biosensor, as described elsewhere herein. The notifications may relate to enabling BLUETOOTH® and alert the user that Bluetooth is required to receive Biosensor readings, and the notification may request that the user turn Bluetooth on now. The notifications may relate to checking their biosensor and alert the user that it looks like their Biosensor is not applied properly. The notifications may relate to replacing the Biosensor, and alert the user that their Biosensor is not working, instructing the user to remove the Biosensor and pair a new one. The notifications may relate to the end of a Biosensor session, alerting the user that their Biosensor session is completed and that it is time to pair a new Biosensor and continue to learn about their glucose levels. The notifications may relate to the Biosensor being ready, and state that Biosensor data is being received and will be displayed automatically. The notification may also invite the user to explore the glucose wellness application. The notifications may relate to the Biosensor ending in a certain number of hours, and request that the user buy a new Biosensor and replace the current Biosensor soon. The number of hours remaining may be 24 hours, 12 hours, 6 hours, 1 hour, or 30 minutes. The notifications may relate to signal from the Biosensor being lost and indicate that the user's phone is out of range of the Biosensor. The notifications may relate to the Biosensor being too hot to give readings, and request that the user check again in a few minutes. The notifications may relate to the Biosensor being too cold to give readings, and request that the user check again in a few minutes. The notifications may relate to a Biosensor error and inform the user that Biosensor reading is unavailable, and request that the user check again in 10 minutes.

### Biosensor Ended/Session Completed

When a biosensor session has ended and it is time to replace the biosensor, the sensor control module 500 may cause a reminder to pair a new biosensor in numerous ways. In some embodiments, as seen in FIGS. 10A-10B, an icon 1008 and a status information message 1010 may appear that reminds the user to start a new biosensor. The icon 1008 for starting a new biosensor may be a different icon than others used regarding the status of the biosensor and may resemble a full moon, a light bulb, etc.

In some embodiments, a pop-up window 1016 may appear, either in addition to the status information message 1010 or in the alternative, and remind the user that the biosensor session has ended and a new biosensor must be paired. In some embodiments, if the life of the biosensor is 14 days, a pop-up winder 1016 may appear when the user has just finished a 14-day session and may say that the biosensor session is completed. The pop-up window 1016 may also remind the user that it is time to replace the biosensor by pairing a new biosensor. The pop-up window 1016 may include also include a selectable “Pair” or “Pair Biosensor” button that, when selected, would display a GUI that assists the user to begin the pairing process. The pop-up window 1016 may also include a link to instructions regarding how to apply the biosensor and/or a link to a website where the user may purchase another biosensor.

In some embodiments, as seen in FIG. 10B, the pop-up window 1016 may appear when the user comes back to the host application but has not set up a new sensor. In some embodiments, the pop-up window 1016 may welcome the user back to the application. The pop-up window 1016 may also remind the user to pair a new biosensor. The pop-up window 1016 may include also include a selectable “Pair” or “Pair Biosensor” button that, when selected, would display a GUI that assists the user to begin the pairing process. The pop-up window 1016 may also include a link to instructions regarding how to apply the biosensor and/or a link to a website where the user may purchase another biosensor. When the user is not browsing the application or when the application is in the background or closed, notifications may appear on the device, e.g., on the lock screen. In some embodiments, the notification may indicate that “Your session is completed!” and suggest that the user pair a new biosensor to keep learning about their body.

The biosensor module details GUI 1100 may also display a reminder to start a new biosensor in as a message associated with graphic 1102, as seen in FIGS. 11A-11B. The message may remind the user to start a new biosensor. The message may also advise the user to please remove the biosensor and pair a new biosensor. GUI 1100 may also include a selectable “Pair” or “Pair Biosensor” button that, when selected, would display a GUI that assists the user to begin the pairing process. In some embodiments, the pop-up window 1120 may appear with a reminder that the new biosensor is ready to scan after the user selects the “Pair” or “Pair Biosensor” button. The pop-up window 1120 may include a graphic of a phone or reader device and may also include instructions reminding the user to hold the top of the phone very close to the biosensor. The pop-up window 1120 may also remind the user that the phone will vibrate or otherwise notify the user (e.g., a sound) after successfully scanning the biosensor.

As mentioned with respect to different GUIs, e.g., the Details GUI, the application may provide a link to “how to replace your biosensor,” which may be helpful to new users. If the user selects the link to “how to replace your biosensor,” a GUI may be presented with selectable options, (1) removing the biosensor, and (2) applying a new biosensor. The GUI may also include an option for replacing the current biosensor before it ends.

If the user wants to remove the biosensor, a GUI may be displayed that includes a warning that the action of removing the biosensor is not reversible. Once the user removes their biosensor, they will need to start a new one. The GUI may also contain instructions to pull up the edge of the adhesive that keeps the biosensor attached to the user's skin and slowly peel away the adhesive from their skin in one motion. If any remaining residue remains on the skin, it can be removed with warm soapy water or isopropyl alcohol. The GUI may also contain instructions for the user to discard the used biosensor according to their local regulations. Furthermore, it may instruct the user that when they are ready to apply a new biosensor, follow the instructions in a provided link to “Apply New Biosensor.” By selecting or tapping the link to “Apply New Biosensor,” the GUIs described in the set-up section may appear.

If the user opts to replace the biosensor before it ends, a pop-up window may appear asking if the user wants to end the biosensor early. If the user taps “END BIOSENSOR,” this will force-end the current biosensor and the user will need to remove it and apply a new biosensor. The pop-up may also contain a warning that this action is not reversible. If the user selects the link to end the biosensor, then the application may display the GUIs associated with replacing the biosensor discussed elsewhere.

After the first biosensor has ended, a pop up may appear that prompts the user to rate the monitoring application.

### Replace Biosensor Due to Sensor Errors

The application may display a pop-up or alert if the biosensor needs to be replaced. The pop-up or alert may include a warning icon (e.g., an orange triangle with an exclamation point) that warns the user that their biosensor is not working and instructs the user to please replace their biosensor and pair the new biosensor. The pop-up may also include selectable links to “pair” or “pair biosensor” or instructions on how to replace their biosensor. The live screen may also include indications that the biosensor is not working properly. Instead of displaying a current analyte level, the banner may display dash marks (e.g., “- -”) instead of a numerical value, and the informational text may state “SEE DETAILS.” If the user taps on any part of the banner, the Details GUI, described elsewhere, may appear with the message to replace their biosensor. As described elsewhere, the Details GUI may include links to additional information. For example, the Details GUI may include a link to how to replace the biosensor, order a biosensor, support, and information about the monitoring application.

### Communication Between Sensor and Applications

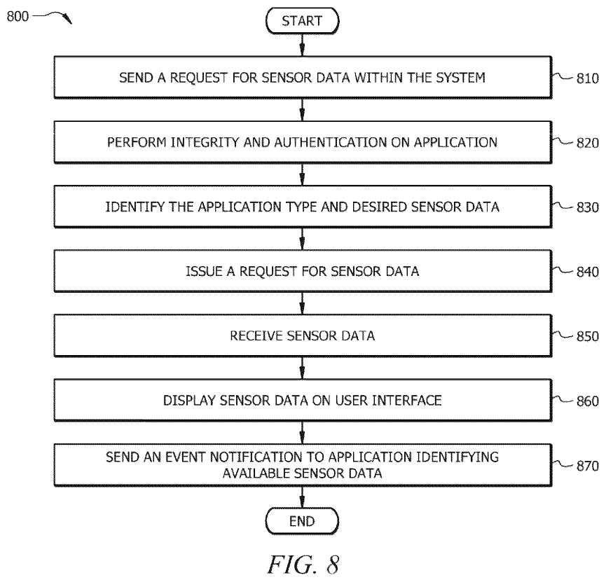

FIG. 8 depicts an example method for communicating sensor data from a sensor to a third-party application 428. As an initial matter, it will be understood by those in the art that any or all of the method steps and/or routines described herein may comprise instructions (e.g., software, firmware, etc.) stored in non-volatile memory of a sensor control device, a remote device (e.g., smartphone, reader), and/or any other computing device that is part of, or in communication with, an analyte monitoring system. Furthermore, the instructions, when executed by the one or more processors of their respective computing device, can cause the one or more processors to perform any one or more of the method steps described herein. Computing device may be the receiving device 200. In addition, although one or more of the method steps and/or routines described herein may comprise software and/or firmware stored on a single computing device, those of skill in the art will recognize that, in certain embodiments, the software and/or firmware may be distributed across multiple similar or disparate computing devices or software modules.

According to one aspect of the embodiments, method 800 can support an application 422, 424, 426 or a third-party application 428 from receiving sensor data for use within the application. At step 810, a third-party application 428 sends a request for sensor data within the system. The request routes to sensor control module 500 through the sensor control module interface 520; the second control module 500 communicates to the sensor assembly 300 using communication control module 540. At step 820, the sensor control module 500 verifies the authenticity of the third-party application 428 and integrity of the session. The sensor control module 500 may further communicate with the remote management module 600 to support user authentication and obtain content specific information for the third-party application 428. These modules may be available within a software library 400 so that a third-party application 428 developer can integrate as a framework within the system of the third-party application 428. At step 830, the sensor control module 500 using logic can identify the third-party application type and desired sensor data.

At step 840, the sensor control module 500 can issue a request for sensor data to the sensor assembly. Alternatively, the sensor control module 500 can be in receipt of sensor data based on a predetermined transmission rate (e.g., every 30 seconds, every minute, every 5 minutes, etc.). According to some embodiments, the sensor data can comprise data indicative of an analyte level, such as, for example, a glucose level, a glucose rate-of-change, a glucose trend, or a glucose alarm condition, among others. At step 850, the sensor data is delivered through a communications link 102 and stored within the database 530 of the sensor control module 500, and displayed at the user interface 510 as shown at step 860.

The sensor control module 500 database 530 includes and can store sensor data separately for each value generated by the various sensor assemblies 300. Database manager 532 may control one or more databases 530 with each separately storing the different types of sensors comprising the sensor assemblies 300. The data may also be stored together within a single database 530. The pictured database 530 is for illustration purposes, not limitation. Separate databases may also be dedicated to storing alarm conditions and triggered alarm results or notifications for each alarm at the sensor control module 500 database 530.

The user interface 510 may also be used to generate alarm notifications to users for alarms that have been triggered based on the sensor data or based on the condition of the sensor assembly 300. The sensor control module 500 may need to alert a user concerning the presence of an alarm. That communication would occur through the sensor control module interface 520 and driven by the user interface 510.

The disclosed subject matter further includes that the remote management module 600 may store alarm notifications and events for the application 422, 424, 426, or third-party application 428 as a backup at the remote server 640. This would allow alarm events to be generated for application 422, 424, 426 or a third-party application 428 that can be stored outside of a module that requires regulatory review and approval. In this manner, different applications developed to monitor a user's health and wellness can use the alarm events for wellness purposes that do not require regulatory clearance. The application 422, 424, 426 or a third-party application 428 may also store sensor data, alarm conditions, or notifications within its own database or shared database separate from database 530 within the sensor control module 500.

As disclosed herein, one such benefit of the software library 400 and modular approach of using the sensor control module 500 is that it would allow users and application developers to identify and develop different wellness related applications for the sensor data. This would enable users that do not traditionally use tracking of analytes, such as glucose monitoring, to consider adding it for purposes of health and wellness such as food tracking, customizable diabetes management, and other unregulated uses. By having the modular sensor control module 500, third-party applications 428 could use the sensor data in any unregulated manner without having to perform the regulatory clearance process. This in turn would expand the user-base for a manufacturer's sensor assemblies 300 by virtue of having more functions available for a user considering using the manufacturer's sensors. Those features can be implemented and improved on these third-party applications 428 without having to submit the revised improvements for regulatory review and clearance, further demonstrating how this disclosure improves initiatives to target wellness for users.

This allows the software library to be expandable to use the sensor control module 500 to collect and provide sensor data for yet to be developed sensors. The modular approach disclosed herein would reduce the need to rewrite code for shared functions and approaches to reading data from the various existing sensors and newly developed sensors, minimizes costs for introducing new sensors, and increases the functions and options for use of that sensor data in a wellness application. The expandable configuration allows the overall system to be extendable to future generations of sensor assemblies 300 and applications of the sensor data to additionally promote wellness use cases. The modular configuration allows third-party application 428 to use a mix and match approach to building and scaling the underlying third-party application 428 and expanding the capabilities offered by third-party application 428. The third-party application 428 can choose which analytes to monitor and incorporate into a wellness program based on the sensor data.

The sensor control module may further, at step 870, issue an event notification to the third-party application 428 identifying that the sensor data is available. The sensor data can be further transmitted using the sensor control module interface 520.

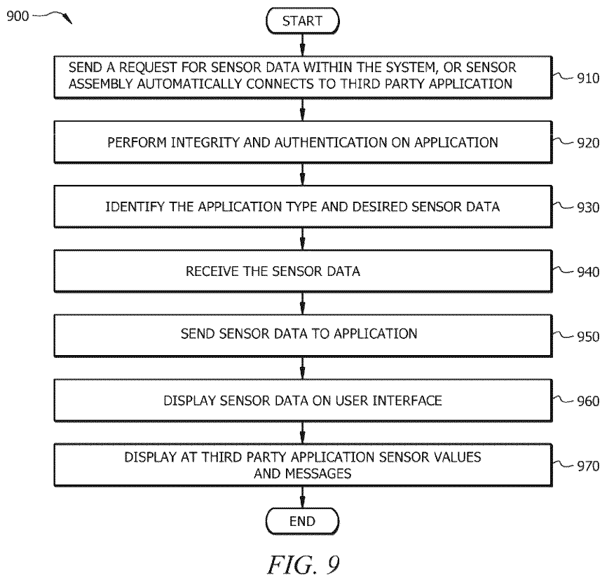

FIG. 9 depicts an example method for communicating sensor data from a sensor to a third-party application 428. As an initial matter, it will be understood by those in the art that any or all of the method steps and/or routines described herein may comprise instructions (e.g., software, firmware, etc.) stored in non-volatile memory of a sensor control device, a remote device (e.g., smartphone, reader), and/or any other computing device that is part of, or in communication with, an analyte monitoring system. Furthermore, the instructions, when executed by the one or more processors of their respective computing device, can cause the one or more processors to perform any one or more of the method steps described herein. Computing device may be the receiving device 200. In addition, although one or more of the method steps and/or routines described herein may comprise software and/or firmware stored on a single computing device, those of skill in the art will recognize that, in certain embodiments, the software and/or firmware may be distributed across multiple similar or disparate computing devices or software modules.

According to one aspect of the embodiments, method 900 can support an application 422, 424, 426 or a third-party application 428 from receiving sensor data for use within the application. At step 910, a third-party application 428 sends a request for sensor data within the system, or the sensor assembly 300 automatically connects to the third-party application 428 using, for example, a BLE connection by issuing a discovery request for BLE capable receiving devices 200 having the third-party application 428. At step 920, the sensor control module 500 verifies the integrity and performs authentication of the third-party application 428. The sensor control module 500 may further communicate with the remote management module 600 to support integrity and obtain content specific information for the third-party application 428. These modules may be available within a software library 400 that a third-party application 428 developer can integrate as a framework within the system of the third-party application 428. At step 930, the sensor control module 500 using logic can identify the third-party application 428 type and desired sensor data to issue requests for the desired data. Optionally, because the session has been authenticated, the sensor assembly 300 may send sensor data through the communication control module 540 and sensor control module interface 520 without a request.

At step 940, the sensor control module 500 receives the sensor data. As stated above, the sensor data can comprise data indicative of an analyte level, such as, for example, a glucose level, a glucose rate-of-change, a glucose trend, or a glucose alarm condition, among others.

At step 950, the sensor data at the sensor control module 500 is sent to the third-party application through the sensor control module interface 520. At step 960, the sensor data is displayed on the user interface 510 of the sensor control module.

At step 970, the third-party application 428 displays any additional messaging related to the sensor assembly 300, including the sensor data relating to analyte levels, notifications, alarms, a message, or other issue regarding the sensors or meal and exercise recommendations based on received sensor data from step 950. Thus, part of the display is via sensor control module 500 regarding analyte levels, whereas another portion of the display on the third-party application 428 is done specifically by the third-party application 428 outside of the control of the sensor control module 500.

The software library 400 and sensor control module 500 as disclosed herein can be used with applications 422, 424, 426. Applications 422, 424, 426 may include various current applications, such as glucose sensor for diabetic monitoring, glucose and ketone sensor for diabetic monitoring, glucose sensor and an insulin delivery device for diabetic monitoring and closed-loop insulin delivery system, and glucose sensor for a wellness application. As disclosed herein, these applications may require various regulated functions and thus need to be submitted in full for regulatory clearance. As disclosed herein, various modifications and functionalities can be added to these applications that do not fall within the core functions for diabetic monitoring and insulin delivery, allowing for unregulated expansion of functions provided by applications based on the sensor data. As further disclosed herein, additional functions can be implemented by applications 422, 424, 426 or third-party applications 428 for wellness, such as glucose sensor for sports or fitness monitoring or for wellness and diet, ketone sensor for wellness or diet plan, such as a keto diet plan, lactate sensor for sports and fitness monitoring, or any number of other applications including alcohol monitoring for treatment and compliance, sST2, Calprotectin, HNL, NT-pro-BNP. Such functionalities can be performed by applications 422, 424, 426 or by third-party applications 428 and reside outside of the core functionality necessary for regulatory review. Thus, enhancements to these functionalities would not need to be submitted for regulatory clearance before introducing the functionalities to the consumer market by use of the modular framework as disclosed herein.

### Alternative Analyte System Configuration

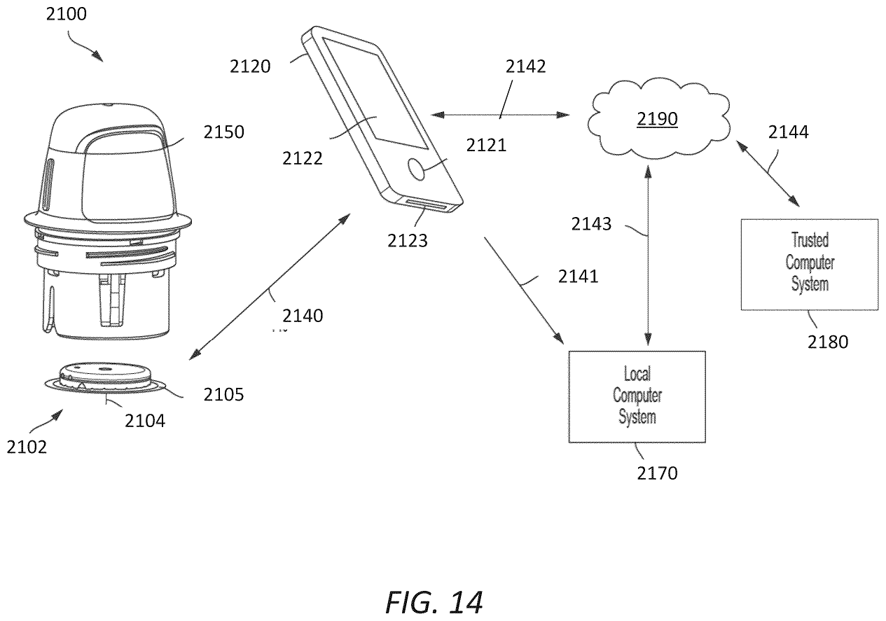

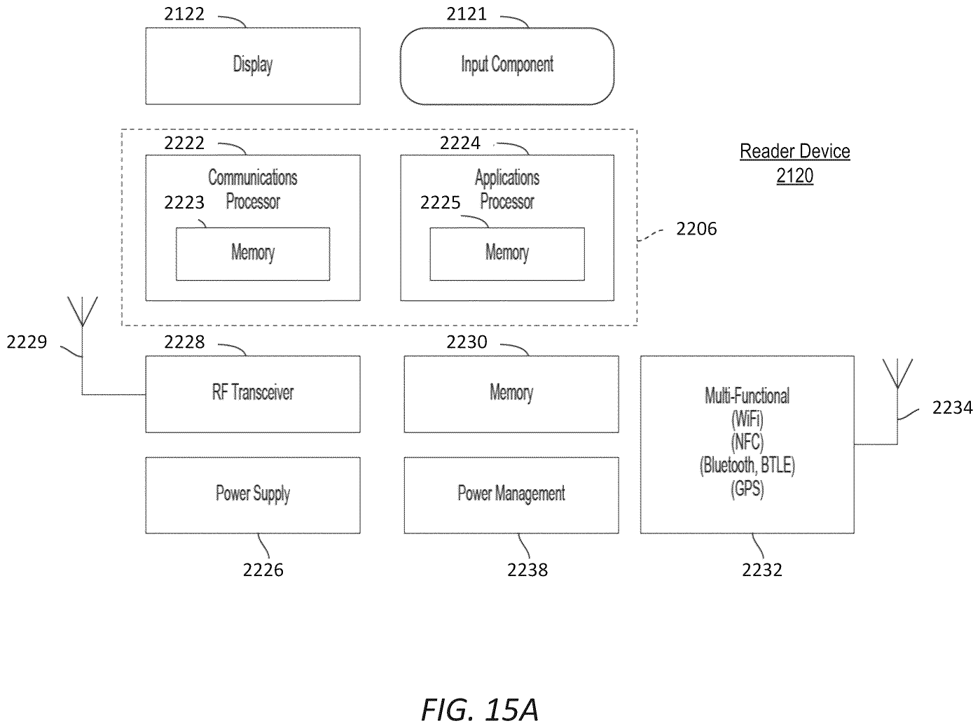

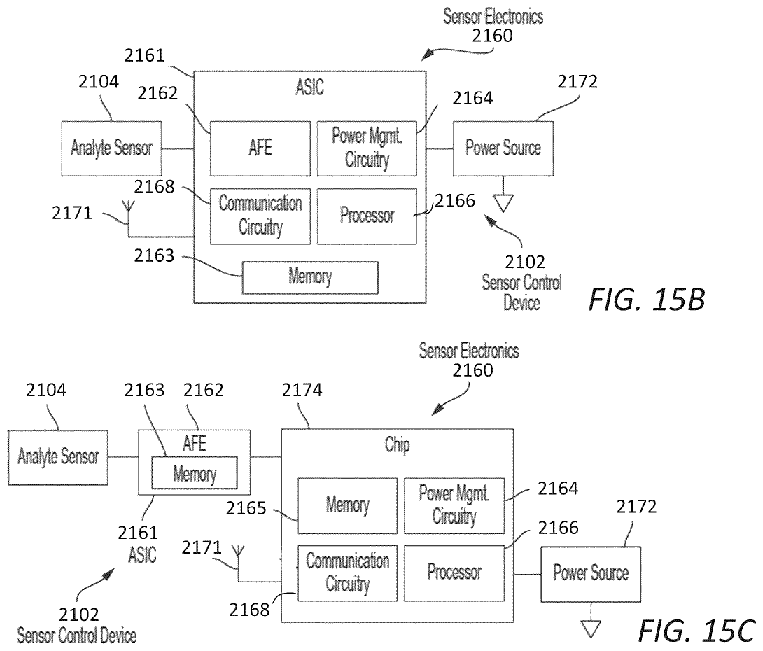

FIG. 14 is a conceptual diagram depicting an example embodiment of an analyte monitoring system 2100 that includes a sensor applicator 2150, a sensor control device 2102, and a reader device 2120. Here, sensor applicator 2150 can be used to deliver sensor control device 2102 to a monitoring location on a user's skin where a sensor 2104 is maintained in position for a period of time by an adhesive patch 2105. Sensor control device 2102, which is further described in FIGS. 15B and 15C, can communicate with reader device 2120 via a communication path 2140 using a wired or wireless technique. Example wireless protocols include Bluetooth, Bluetooth Low Energy (BLE, BTLE, Bluetooth SMART, etc.), Near Field Communication (NFC) and others. Users can view and use applications installed in memory on reader device 2120 using screen 2122 (which, in many embodiments, can comprise a touchscreen), and input 2121. A device battery of reader device 2120 can be recharged using power port 2123. While only one reader device 2120 is shown, sensor control device 2102 can communicate with multiple reader devices 2120. Each of the reader devices 2120 can communicate and share data with one another. More details about reader device 2120 is set forth with respect to FIG. 15A below. Reader device 2120 can communicate with local computer system 2170 via a communication path 2141 using a wired or wireless communication protocol. Local computer system 2170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 2170 can communicate via communications path 2143 with a network 2190 similar to how reader device 2120 can communicate via a communications path 2142 with network 2190, by a wired or wireless communication protocol as described previously. Network 2190 can be any of a number of networks, such as private networks and public networks, local area or wide area networks, and so forth. A trusted computer system 2180 can include a server and can provide authentication services and secured data storage and can communicate via communications path 2144 with network 2190 by wired or wireless technique.

### Example Embodiment of Reader Device

FIG. 15A is a block diagram depicting an example embodiment of a reader device 2120, which, in some embodiments, can comprise a smartphone. Here, reader device 2120 can include a display 2122, input component 2121, and a processing core 2206 including a communications processor 2222 coupled with memory 2223 and an applications processor 2224 coupled with memory 2225. Also included can be separate memory 2230, RF transceiver 2228 with antenna 2229, and power supply 2226 with power management module 2238. Further, reader device 2120 can also include a multi-functional transceiver 2232, which can communicate over Wi-Fi, NFC, Bluetooth, BTLE, and GPS with an antenna 2234. As understood by one of skill in the art, these components are electrically and communicatively coupled in a manner to make a functional device.

### Example Embodiments of Sensor Control Devices

FIGS. 15B and 15C are block diagrams depicting example embodiments of sensor control devices 102 having an analyte sensor 2104 and sensor electronics 2160 (including analyte monitoring circuitry) that can have the majority of the processing capability for rendering end-result data suitable for display to the user. In FIG. 15B, a single semiconductor chip 2161 is depicted that can be a custom application specific integrated circuit (ASIC). Shown within ASIC 2161 are certain high-level functional units, including an analog front end (AFE) 2162, power management (or control) circuitry 2164, processor 2166, and communication circuitry 2168 (which can be implemented as a transmitter, receiver, transceiver, passive circuit, or otherwise according to the communication protocol). In this embodiment, both AFE 2162 and processor 2166 are used as analyte monitoring circuitry, but in other embodiments either circuit can perform the analyte monitoring function. Processor 2166 can include one or more processors, microprocessors, controllers, and/or microcontrollers, each of which can be a discrete chip or distributed amongst (and a portion of) a number of different chips.

A memory 2163 is also included within ASIC 2161 and can be shared by the various functional units present within ASIC 2161, or can be distributed amongst two or more of them. Memory 2163 can also be a separate chip. Memory 2163 can be volatile and/or non-volatile memory. In this embodiment, ASIC 2161 is coupled with power source 2172, which can be a coin cell battery, or the like. AFE 2162 interfaces with in vivo analyte sensor 2104 and receives measurement data therefrom and outputs the data to processor 2166 in digital form, which in turn processes the data to arrive at the end-result glucose discrete and trend values, etc. This data can then be provided to communication circuitry 2168 for sending, by way of antenna 2171, to reader device 2120 (not shown), for example, where minimal further processing is needed by the resident software application to display the data.

FIG. 15C is similar to FIG. 15B but instead includes two discrete semiconductor chips 2162 and 2174, which can be packaged together or separately. Here, AFE 2162 is resident on ASIC 2161. Processor 2166 is integrated with power management circuitry 2164 and communication circuitry 2168 on chip 2174. AFE 2162 includes memory 2163 and chip 2174 includes memory 2165, which can be isolated or distributed within. In one example embodiment, AFE 2162 is combined with power management circuitry 2164 and processor 2166 on one chip, while communication circuitry 2168 is on a separate chip. In another example embodiment, both AFE 2162 and communication circuitry 2168 are on one chip, and processor 2166 and power management circuitry 2164 are on another chip. It should be noted that other chip combinations are possible, including three or more chips, each bearing responsibility for the separate functions described, or sharing one or more functions for fail-safe redundancy.

### Glucose Wellness Application

During the day, a person's blood glucose rises and falls many times. A glucose spike, however, is a sharp, marked rise in the amount of glucose in a person's blood, followed by a comparable decline. In a glucose vs. time graph, a spike may appear as a tall mountain, not a hill or a steady, flat plain. Most people get a rise in glucose after a meal, but the type of food consumed, stress levels, exercise levels, and the person's metabolic health can affect the speed and amount of the rise in levels. The wellness application may help the user understand what caused a spike and how to manage and prevent spikes.

After eating, it can take up to 90-120 minutes before a spike may occur. The wellness application allows a user to look at the last meal before the spike. Many people may see two peaks in their spike after a meal, one around about 30 minutes after eating and another around about 90 minutes after eating. This can indicate good metabolic fitness.

Post-prandial hyperglycemia (PPHG) is a sharp rise in plasma glucose concentrations following food intake and is influenced by many factors including the timing, quantity and composition of a consumed meal. The state of post-prandial hyperglycemia begins when plasma glucose rises above the level of 140 mg/dL (7.8 mmol/L), 1-2 hours after ingestion of food in individuals without Diabetes Mellitus (DM) and >180 mg/dL (10.0 mmol/L) in individuals with DM. A degree of fluctuation in blood glucose is common after food intake, especially with meals containing carbohydrate, but broadly speaking, in individuals without DM, glucose levels peak approximately 1 hour after the start of a meal and return to baseline within 2-3 hours. Most meals peak below 140 mg/dl. Comparatively, in individuals with DM, glucose levels frequently exceed 180 mg/dl following food ingestion, and the rate of restoration to baseline is dependent upon the subject's diabetic control and treatment regime. See Jarvis, P. E. et al., “Continuous Glucose Monitoring in a Healthy Population: Understanding the Post-prandial Glycemic Response in Individuals without Diabetes Mellitus.” 10.31219/osf.io/mdrpt, which is expressly incorporated by reference in its entirety for all purposes.

Healthy people have glucose values in the range of 70 mg/dL-140 mg/dL for about 23 hours a day. Occasional variations above and below are expected. The more time that a person is in the target range (70 mg/dL-140 mg/dL), the better the person may feel. With the glucose wellness application, the person will likely see spikes in their glucose. A spike or excursion is a sharp rise in the amount of glucose in the blood. It is often followed by a sharp decline. The user may find themselves going over 140 or under 70 mg/dL, which is okay. It is normal to be out of range for 30 minutes to 2 hours a day. Spikes can be brief or last several hours. The spikes can take different shapes, such as sharp and tall, have multiple peaks, or look like a plateau. Occasionally, a person's body will overcorrect and release too much insulin, which is sometimes called a crash. Glucose crashes sometimes result in cravings or drowsiness. Common causes of spikes include: meals, snacks and drinks, exercise, stress, and disrupted sleep. Over time, spikes damage your metabolism as they trigger insulin and can lead to insulin resistance, a cause of pre-diabetes, diabetes, and other metabolic problems. Minimizing spikes (smaller and fewer spikes) is how a person can improve their health and metabolism.

Spikes may also sometimes appear outside of the graph's visible range. This can be caused by long-duration, high-intensity exercise or long-duration, high-intensity exercise combined with a meal high in carbohydrates. Exercise may cause the user to spike because muscles need glucose.

In some embodiments, a wellness application that monitors glucose levels in a subject or user may communicate with the sensor control module 500 to obtain glucose data. In other embodiments, the wellness application may communicate with the sensor control device to obtain glucose data. The wellness application may receive glucose data in “real-time,” i.e., in 5-minute increments, as they become available from the sensor or cloud. In some embodiments, the wellness application may also receive or be capable of receiving back-fill data that becomes available upon sensor reconnection. From this data, both current spike status as well as prior spikes may be provided to the user. The algorithm in the wellness application may remain functional during disconnections from the sensor or cloud, filling in missing data upon reconnection. The user may not have diabetes but may want to monitor their glucose levels to improve their health. When a user first opens or logs into the glucose wellness application, a series of GUIs may appear to customize the user's experience and assist the user in making goals for the glucose wellness application to monitor.

The wellness application may be connected to and receive data from other applications or wearable devices. In some embodiments, the application may be synchronized with a health application, such as Apple Health, and incorporate a user's profile information to assist in the set-up process. In some embodiments, the wellness application may receive at least one of a date of birth, height, sex, weight, and workouts from a connected health application. In some embodiments, the user may select and approve which of a plurality of data will be accessed by the wellness application. In some embodiments, a GUI may be displayed in the wellness application that asks the user to confirm and/or edit information obtained from the health application, such as date of birth, height, weight, and sex.

The wellness application may include a counts system. A user may be assigned a daily budget of counts for a time period, with a goal or reducing the daily budget or daily counts goal over time through diet and lifestyle changes. When the user experiences a glucose spike, or a spike or peak in the user's glucose levels (or blood sugar), an amount of counts will be assigned to the spike based on a glucose metric determined for the glucose spike.

A home screen or live screen of the wellness application may display the user's daily counts budget along with the user's current counts score for the day. The first daily budget assigned may be a general starting budget number assigned to all users. In some embodiments, the first daily budget assigned may be assigned based on the user's age. For example, the first daily budget may be assigned based on an age group in which the user falls, such as 51-60 year olds. In the following weeks, a new daily counts budget may be determined. In some embodiments, the new daily counts budget may be determined based on the total counts spent during an assessment period or the previous week. In some embodiments, the new daily counts budget may also be determined based on a comparison to a population of other users with similar total daily counts scores. Such a comparison to a population of other users with similar total daily counts scores may be helpful in determining a new daily counts budget because users that spend a higher number of counts may experience more improvements when using the wellness application in the beginning than users who spend a lower number of counts. Machine learning models may be used to best match users to other users like them for a more accurate prediction of user outcomes and the advice to which they may respond.

The home or live screen may also display a background color that reflects the current state of the user's glucose, e.g., whether or not they are experiencing a spike or not. Moreover, if the user is experiencing a spike, a first color may be displayed if the spike is rising, while a second color may be displayed if the spike is declining, and a third color may be displayed if the user is flat but still in the spike. The color displayed may be determined by calculating a glucose trend status based on the glucose counts assigned. In some embodiments, different colors may appear when a user is spiking. A first color may be used to indicate that the user is spiking and accruing counts at a slower rate. A different color may be used to indicate that the user is spiking and accruing counts at a faster rate.

The live or home screen may also display text reporting the current state, such as stating that the user is steady, the user is coming off a glucose spike, or the user's glucose is spiking. In some embodiments, the live or home screen may also display a recommendation related to the user's current state. In some embodiments, the live or home screen may also display recommendations related to reducing their glucose counts.

The wellness application may also be configured to receive and track logged events, such as meals, exercise, stress, and sleep. In some embodiments, the wellness application may also prompt the user to provide input regarding events. The prompts may be an alert, an icon with a question mark in a graph, or a list of untracked events. The live or home screen may also include a graph of the glucose levels vs. time for the current day. Logged events may also appear as icons along the graph according to their respective time stamps. Icons with a question mark or other alert may also appear on the graph at the start of a glucose spike if there is no logged event with that glucose spike.

The wellness application may also provide a periodic report, e.g., a weekly report, which summarizes the user's count totals for the various days. The periodic report may identify a focus area on which the user may concentrate. The focus area may be a time-of-day period in which the user spent the most counts during the time period covered by the periodic report. The periodic report may also display the average total counts spent each day relative to the total counts goal or budget. The periodic report may also include a graphical display of the average amount of counts spent in each time-of-day period. The graphical display may also be color-coded to highlight the time-of-day period when the most counts were spent or where the most glucose spikes occurred.

Recommendations and tips in the wellness application may be centered around fundamental principles. The principles may include prioritizing proteins, fueling with healthful fats, choosing more non-starchy vegetables during the day, starting all meals with vegetables, choose savory foods over sweet foods to reduce overall carbohydrate intake, and move and/or exercise more. The recommendations and tips may also encourage the user to cat foods in a preferred order. The ideal order for a steady glucose level is vegetables first, proteins and fats second, starches (bread, pasta, rice, potatoes), and sugars last. The recommendations and tips may also include prioritizing protein, which is known to balance blood glucose levels, reducing hunger, repairing and rebuilding muscle, and supporting the immune system. The recommendations and tips may also include fueling with healthy fats instead of carbohydrates.

### On-Boarding

During setup, the glucose wellness application may ask the user to identify their priority in using the application, such as more energy, managing hunger, or better mood. A GUI presenting this question may optionally include a text box for the user to input a different goal or priority. The list of possible options may be modified to include additional goals that were received from various user input.

The glucose wellness application may also prompt the user to describe their hunger in the past 24 hours. A GUI may be displayed that includes a slider with a button where the user can set the button between low and high.

The glucose wellness application may also ask the user when they usually cat. The application may ask for time ranges in which the user usually eats breakfast, lunch, dinner, and snacks. The user may also have the option to add time ranges for additional meals or snacks. Timing is important to keeping glucose levels or blood sugar in check or within a target range. The glucose wellness application may give the user tips on how to cat to reap the biggest glucose benefits.

The glucose wellness application may also ask the user what they normally cat in a day. A GUI may display breakfast, lunch, dinner, and meals/snacks options that the user may select as applicable. After clicking continue, a GUI may appear for the user to enter meal-time scheduling, where the user may pick a time frame that applies to their normal eating pattern. For each of the options selected (breakfast, lunch, dinner, and meals/snacks before bed), inputs for beginning and ending times may be displayed. The inputs may be text boxes in which the user may type in times. Alternatively, the inputs may be wheels that can be dialed to the correct times, or sliding scales in which the user can select the correct time ranges.

The glucose wellness application may also ask the user how many hours that the user spends sitting in a day. It may present options of 1-2 hours, 3-4 hours, 5-6 hours, and 7 or more hours.

The glucose wellness application may also ask the user when they usually go to sleep and wake up and display a prompt for the user to enter a time range for bedtime and waking up.

The glucose wellness application may also ask the user if they follow any specific diets. Such information may be used to present relevant tips and suggestions in line with the diets that the user is following. A GUI may appear that lists a plurality of diet options, from which the user can select appropriate diet(s). The list may include keto, low carb, gluten-free, vegetarian, vegan, and none of the above. The GUI may also include a text box in which the user may enter a diet that is not on the list. The list may be modified to include additional diets that were received from various user input.

The glucose wellness application may also ask the user which foods they crave the most. The answer options may be salty and crunchy (e.g., pretzels), sugary (e.g., candy, cookies), savory and greasy (e.g. fries, pizza), fatty (e.g., ice cream, fried chicken), or none of the above.

The glucose wellness application may also ask the user what is their go-to snack. The answer options may include veggies, nuts or nut butter, something salty and crunchy, fruits, something sweet and sugary.

They glucose wellness application may also ask the user about their cooking habits. The answer options may include mostly cooking their own meals, mostly getting meals delivered, mostly eating out at restaurants, or none of the above.

The glucose wellness application may also ask the user to select one or more benefits, goals, or focus area from a list of benefits, goals, or focus areas on which the user wishes to focus. These may include, but are not limited to, boost energy, manage hunger, improve mood, better sleep, and stay focused. Each benefit may be accompanied by an emoji. Boost energy may be selected if glucose spikes leave the user drained and the user wishes to learn to stay steady and energized with the glucose wellness application. Manage hunger may be selected if the user's glucose and hunger is unsteady and the user wishes to manage both their glucose and hunger with glucose tracking with the glucose wellness application. Improve mood may be selected if the user wishes to steady their glucose and mood and the user wishes to manage their glucose to live a better life. Better sleep may be selected if the user wishes to improve their amount of sleep or quality of sleep. The glucose wellness application will track their glucose during the day to help them sleep soundly at night. Stay focused may be selected if the user wishes to understand their glucose to help manage their glucose crashes and mental fatigue. If the user wishes to change the benefit that is currently selected, the user may change the selected benefit in the settings.

The glucose wellness application may also ask how the user's energy was over a period of time. The period of time may be, but is not limited to, the last week, last 24 hours, last 12 hours, last 8 hours, last 4 hours. The glucose wellness application may present options regarding the energy level over the period of time. The energy level options may include, but are not limited to, very good, good, neutral, bad, and very bad. The options may be selectable or included in a picklist and be listed with a corresponding emoji.

The glucose wellness application may also request that the user enable notifications. If notifications are enabled, the user will be sent alerts, advice, and sound and icon badges as they use the application. These may include alerts or notifications when the user is spiking, or experiencing a glucose spike, the availability of a daily briefing, and the availability of a weekly report.

### Glucose Count System

According to one aspect of some embodiments, a glucose wellness application can include a glucose count system configured to determine and display a metric associated with a user's glucose exposure. The count system is intended to help the user assess the impact of glucose excursions or spikes on their metabolism. The count value assigned to each excursion or spike is calculated based on the rise and duration of the spike. An algorithm used to determine the count value may consider rise or height of the spike or excursion, the rate of change, and the overall glucose value over baseline. The count system focuses on the spikes or excursions that are most meaningful for the user's metabolism. Smaller variations in glucose are not taken into account. The count system is intended to translate the user's glucose exposure in a simple, actionable way. Different spike shapes can result in the same count value. Additionally, the user's body may process glucose differently at different times. The user is also encouraged to keep track of what makes them spike (i.e., have a glucose excursion), log those events, and see how their counts change over time. The glucose wellness application also tracks what keeps the user's glucose steady and helps the user stick to those habits. Logging intense exercise removes the count assigned to the spike for that event. Over time, the user should see fewer spikes and their glucose should stay in range longer. Staying steady will help the user build better habits. When the user is in control of their glucose, they will experience fewer spikes, feel more energized, and sleep better.

Glucose variability (% CV) is a measure of how much the user's glucose fluctuates from their baseline. Most healthy people have a % CV of 11-23%. A higher count correlates with a higher % CV. All glucose fluctuations, large or small, contribute to % CV.

A user will get a daily budget of counts, with a goal to stay on or below the daily target budget each day, also referred to as a count goal or target count goal. The overall goal is to reduce the daily budget of counts or target count goal over time. The user's daily target may evolve with the user's journey. When there is a spike in a user's blood sugar, the application will assign counts to the glucose spike. These counts will be added into a user's daily score. The lower the score that a user has, the better. An aim of the application is for the user to keep their daily counts total within their counts target each day. The user will hopefully stay within the target amount, but also use their counts. In some embodiments, users may have the option to adjust their count goal to make the daily goal either higher (easier to achieve) or lower (harder to achieve). A user is also encouraged to track events as they happen to enable the user to see what causes the spikes, which will give the user a better understanding of their personal relationship with glucose, and will help the user get to a lower counts score over time. At the end of each week, the user may get a report that summarizes how the user did the previous week, along with including suggestions on what to focus on to continue lowering their counts budget.

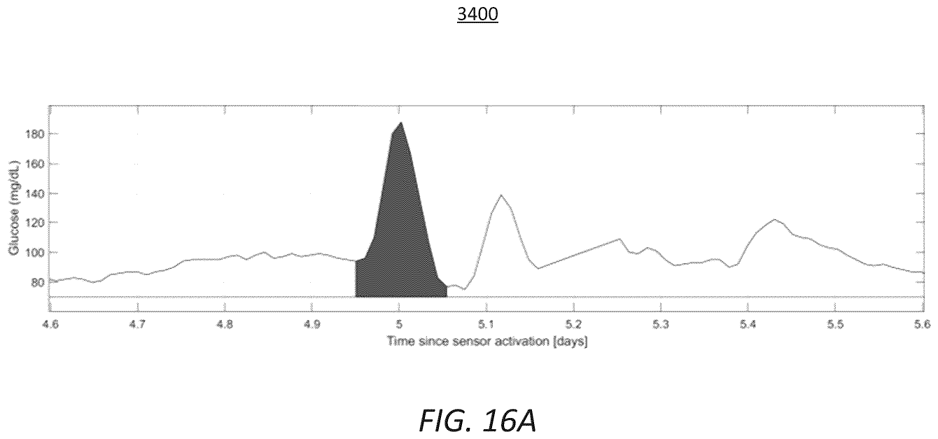

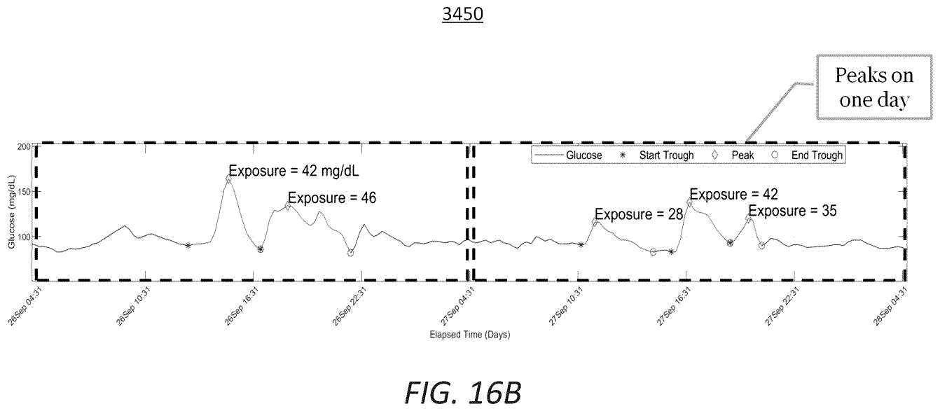

By way of background, glycemic load is the theoretical cumulative exposure to glycemia over a period of time. As can be seen in graph 3400 of FIG. 16A, the measurement of glycemic response can be based on the incremental area under a glucose response curve (IAUC), or “glucose spike,” for a predetermined period of time (e.g., two to three hours) after consuming food. A glucose spike or excursion may be a rapid and sustained increase in glucose levels, followed by a comparable decrease. According to an aspect of the embodiments, each glucose spike can have a starting trough, peak, and an ending trough. Glucose (or glycemic) exposure may be the cumulative exposure to glucose over a period of time. In some embodiments, the measurement of glucose exposure may be based on the size of the spike or excursion, and may be based on at least an area under the curve and a time duration of the curve. In some embodiments, the measurement of glucose exposure may be based on the area under the curve divided by a time duration of the spike portion of the curve or a derivative thereof. As can be seen in graph 3450 of FIG. 16B, glucose exposure can be calculated on a daily basis by identifying the troughs and peaks in the analyte data for a twenty-four-hour period, then calculating the incremental areas under each glucose spike for the twenty-four-hour period. In some embodiments, the calculated glucose exposure may be converted to counts. Counts may be an index value converted from glucose exposure based on a population scale. In some embodiments, the population scale may be a universal population scale. A daily counts value may be determined, which may be an aggregated count value across all glucose spikes for a day.

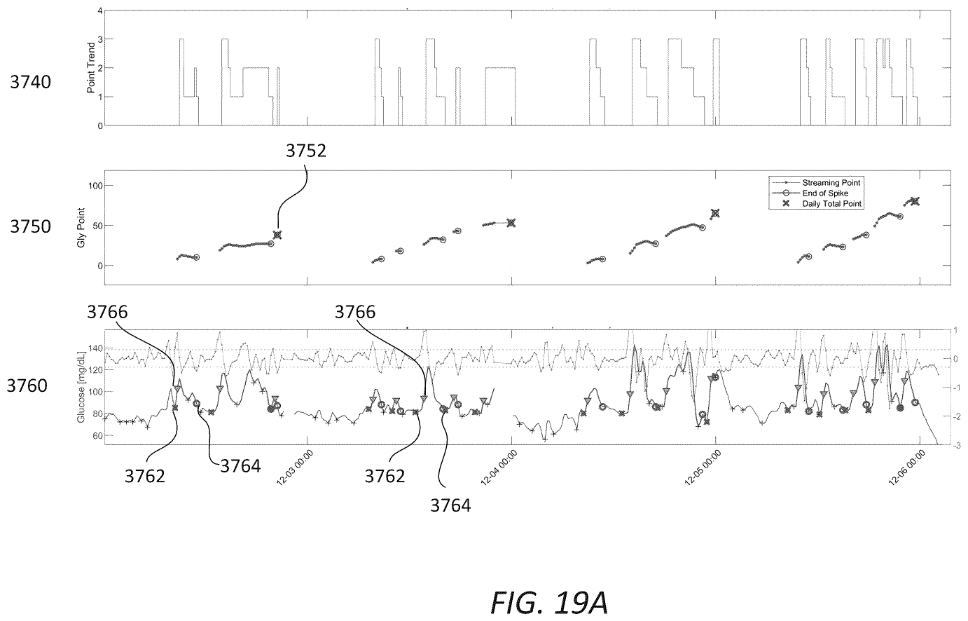

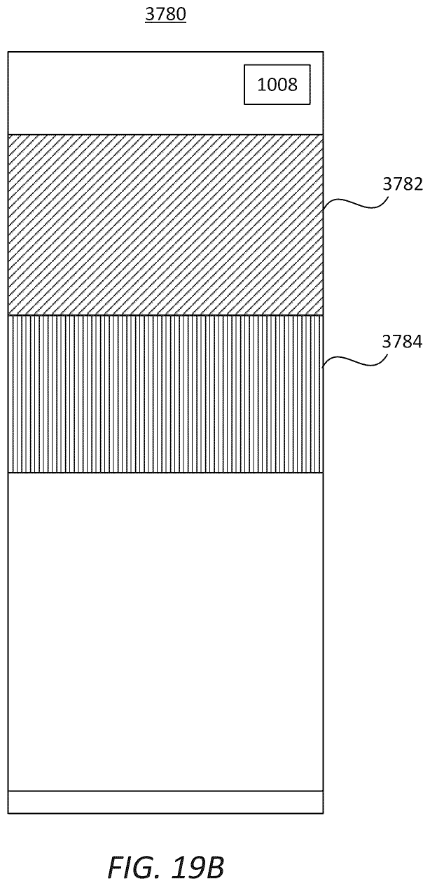

As seen in FIG. 19A, in graph 3760, which shows a graph of glucose vs. time, start counts 3762 and end counts 3764 may be identified as described herein. Moreover, alert counts 3766 may also be identified. After an alert point 3766 in a spike is detected, a notification regarding the spiking condition or that a spike is occurring may be output to the user.

In some methods, once a spike is detected, a glycemic metric, such as area under the curve, or integrated area under the curve over time, may be calculated for the spike, a portion of the spike, or the whole spike. In some embodiments, the glycemic metric may be calculated in parts as the spike is occurring. Once the glycemic metric is calculated for the spike or portion of the spike, a count value may be assigned to that spike or portion of the spike based on the calculated glycemic metric. The count value may be assigned based on a comparison to a distribution of the glucose metric determined from a predetermined population. Graph 3750 of FIG. 19A shows the count values assigned to the various spikes identified in graph 3760. A daily count total 3752 may be calculated, which may be a summation of the glucose counts from the spikes that occurred that day.

A count trend status may also be calculated. The count trend status may be determined by comparing a count value with the preceding count value. If there is no difference in values and the count values are 0, a count trend status may be determined to be balanced, or not in a spike. If the count value is less than the preceding count value, than the count trend status may be determined to be declining in a spike. If the count value is greater than the preceding count value, than the count trend status may be determined to be rising in a spike. If there is no difference in values, and the count values are greater than 0, a count trend status may be determined to be flat during a spike. Graph 3740 of FIG. 19A depicts the count trend status values for each of the peaks below in graph 3760. Alternatively, the count trend status may be determined by determining a slope of a line formed from a plurality of count values. If the slope is positive, then the count trend status may be determined to be rising in a spike. A color representing the count trend status may differ depending on the magnitude of the slope. If the slope is negative, then the count trend status may be determined to be declining in a spike. If the slope is constant or substantially constant, then the count trend status may be determined to be flat in a spike.

Example embodiments of a glucose count system for monitoring and managing a user's glucose exposure, and methods relating thereto, will now be described. As an initial matter, those of skill in the art will recognize that the method steps described herein can comprise software instructions stored in a memory of a computing device of system 100 (e.g., a reader 120, a local computer system 170, a trusted computer system 180), such that the instructions, when executed by one or more processors of the computing device, cause the one or more processors to perform any or all of the method steps described herein.

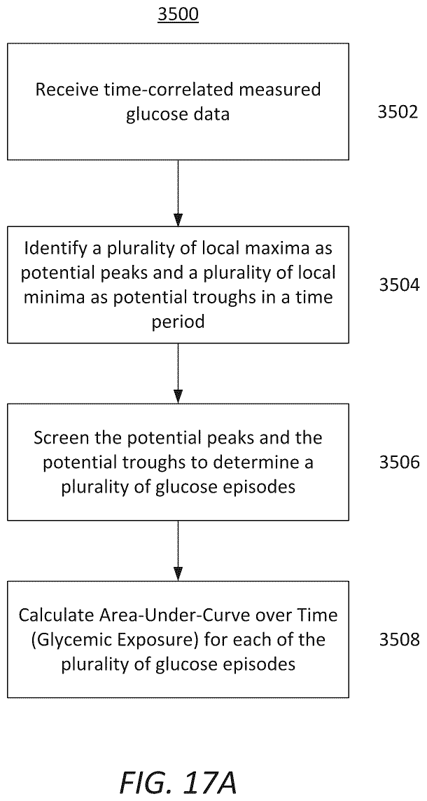

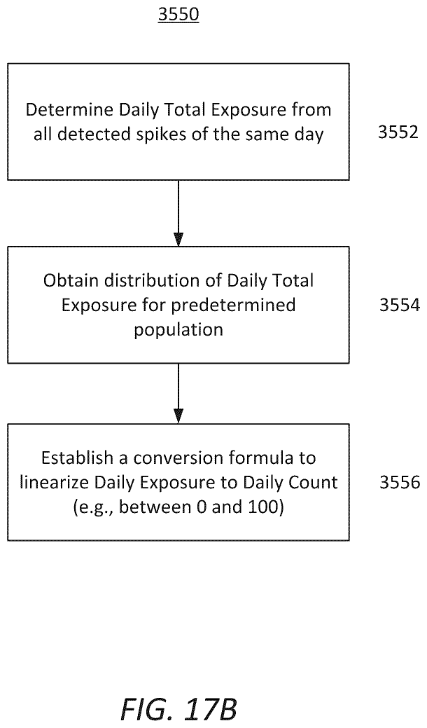

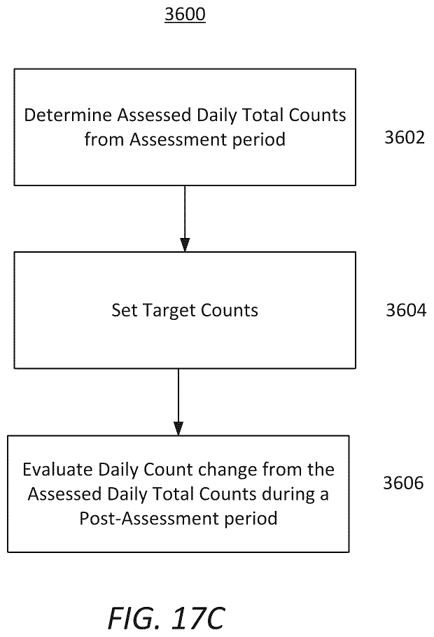

Turning to FIG. 17A, a flow diagram depicts an example embodiment of a method 3500 for calculating glucose exposure. At Step 3502, time-correlated measured glucose data is received. According to some embodiments, the data can be received by a reader 120, local computer system 170, trusted computer system 180, or any computing device used in conjunction with an analyte monitoring system.

At Step 3504, a plurality of local maxima are identified as potential peaks and a plurality of local minima are identified as potential troughs in a time period.

At Step 3506, the potential peaks and the potential troughs are screened to determine a plurality of glucose episodes. One or more algorithms may be used to screen the potential peaks and potential troughs. The one or more algorithms may be applied to screen, merge, eliminate, and/or filter the received data to determine potential peaks and troughs that define glucose episodes.

According to some embodiments, the one or more algorithms can include merging certain adjacent peaks as one peak (e.g., if there are two glucose peaks within a predetermined minimum amount of elapsed time, the maximum glucose value can be selected), and then finding an ending trough for each peak, then screening, merging, and removing peak-troughs of shorter duration, with low rate of change, and missing data.

In some embodiments, the one or more algorithms can include calculating the area under each peak-trough curve (effective drop), and selecting those peak-troughs that meet one or more predetermined area conditions.

In addition, in some embodiments, after each ending trough is found, the one or more algorithms can include a subsequent step of identifying a starting trough for each peak, then screening, merging, and removing trough-peaks of shorter duration, low rate of change, and missing data.

In addition, according to some embodiments, the one or more algorithms can include combining beginning troughs with peak to ending troughs, and removing trough-troughs with low rates of change.

In some embodiments, the one or more algorithms can include calculating the areas under the starting trough to peak (effective rise) and starting trough to ending trough (effective change) for each glucose curve, and selecting those trough-troughs with glucose exposure that meet one or more predetermined change conditions.

At Step 3508, a glycemic exposure value for each of the plurality of glucose episodes determined. According to some embodiments, this is computed as the integral area under trough-peak-trough curve divided by the time duration.

Those of skill in the art will recognize that each of the one or more algorithms described above are optional, and can be applied in any order and/or combination. It will further be understood by those of skill in the art that any of the one or more algorithms can be performed iteratively. In addition, the one or more algorithms described herein are not meant to be limiting, and other algorithms can be implemented to identify qualified spikes, and are considered within the scope of the present disclosure. Additional details can be found, for example, in U.S. Publ. Nos. 2020/0105397 and 2022/0059215, which are hereby incorporated by reference in their entireties for all purposes.

Turning next to FIG. 17B, a flow diagram depicts an example embodiment of a method 3550 for determining a conversion formula for converting glucose exposure values to counts. At Step 3552, a daily total exposure is determined by calculating a rolling sum of glucose exposure for all detected spikes in the same day (e.g., twenty-four-hour period). This step may be performed for each person in the sample population. At Step 3554, a distribution of daily total exposure for a predetermined population (e.g., non-diabetics) is obtained.

At Step 3556, a conversion formula to linearize the daily exposure values to a daily point range is established. According to some embodiments, for example, the daily exposure values of a main portion of a population distribution (e.g., 0 to 90th percentile) can be linearized to a range of daily glucose counts (e.g., between 0 and 100 counts). In some embodiments, for example, the glucose counts can comprise a continuous system from 0 to 100 counts, with intervals of one. In addition, according to some embodiments, for daily exposure values above the 90th percentile, a point value can still be calculated using the same conversion formula—that is, with a point value above 100—but can be capped at a predetermined maximum point value (e.g., 500 counts). This would allow the point system to be used by pre-diabetic and diabetic users, or individuals with high glycemic exposure.

Those of skill in the art will recognize that the conversion formula described above is meant to be an illustrative embodiment, and that other conversion formulas or point systems can be implemented in addition to, or in lieu of, the conversion formula described above, and are fully within the scope of the present disclosure. Additional details of count systems can be found, for example, in U.S. Publ. No. 2018/0256103, which is hereby incorporated by reference in its entirety for all purposes.

Once a conversion formula is established, the formula may be used to convert any glucose exposure value to a count value. Because the conversion formula is linear, the formula can be applied to any glucose exposure value (e.g., the value of a single spike). In some embodiments, the formula may be used to convert a full-day's-worth of glucose exposure values. In other embodiments, the formula may be used to convert a smaller subset of glucose exposure values. For example, the formula may be used to assign a count value to each spike, and the count value of the spike could allow the glucose wellness application to provide certain messages or warnings. In some embodiments, the counts in a time period (e.g., a day) may also be aggregated to generate a count total for the time period.

Turning next to FIG. 17C, a flow diagram depicts an example embodiment of a method 3600 for assessing the daily count performance of an individual. At Step 3602, a baseline Daily Glucose Counts is determined during an assessment period. In many embodiments, the assessment period can comprise the first seven days of the first sensor wear. According to some embodiments, an initial category (e.g., recruit, novice, apprentice, intermediate, advanced, master, etc.) can be assigned to the individual based on a population scale. The total daily count value for each day during the assessment period may be computed using the methods described above. In some embodiments, in each day, qualifying glucose spikes may be identified. The computing device may then compute glucose exposure values for each spike and convert the glucose exposure value to a count value using the conversion formula. The counts assigned to all of the qualifying spikes in the day may then be added together to derive a daily counts total. In some embodiments, the daily counts total of each day of the assessment period may be averaged to compute a baseline for the user's daily counts total.

At Step 3604, a target count value can be set. In some embodiments, the target count value can comprise a target daily total counts value. In some embodiments, the target count value may be based on the baseline computed in Step 3602. For example, the target count value (or daily count budget), may be set to a value that is lower than the baseline in order to encourage the user to improve his glucose exposure. In some embodiments, a challenge may be presented to the user. If the user successfully met their target for a period of time, the wellness application may issue a challenge to reduce the daily target. If the user accepts the challenge, the target daily budget will be reduced.

At Step 3606, after the assessment period, the daily count change from the assessed daily total counts is evaluated during a post-assessment period. In some embodiments, the evaluation can be a comparison of each daily count total against the target counts to assess the user's progress. In some embodiments, in addition to comparing the daily count totals, the computing device may also display or output the user's mid-day progress by summing the counts assigned to every qualifying spike detected thus far in the day. This allows the user to see their current progress relative to the target. In addition, according to some embodiments, the post-assessment period can comprise an eight-week wear time. In this regard, an individual can track his or her progress with respect to reducing his or her glucose exposure over a longer period of time.

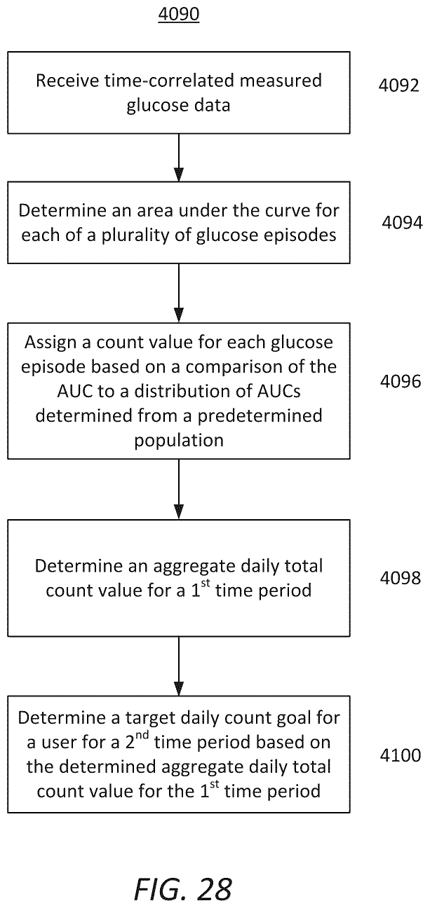

Turning next to FIG. 28, a flow diagram depicts an example embodiment of a method 4090 for assessing a new target daily count goal for a new time period. At Step 4092, time-correlated measured glucose data is received. According to some embodiments, the data can be received by a reader 120, local computer system 170, trusted computer system 180, or any computing device used in conjunction with an analyte monitoring system.

At Step 4094, an area under the curve for each glucose episode of a plurality of glucose episodes in in a dataset of time-correlated glucose data is determined. In some embodiments, an area-under-the-curve over time or an integrated area-under-the-curve over time may be determined.

At Step 4096, a count value for each for each glucose episode of the plurality of glucose episodes may be assigned based on a comparison of the determined area under the curve to a distribution of areas under the curve determined from a predetermined population. In some embodiments, the predetermined population may be a population of users having an aggregate daily total count value within a threshold of the determined aggregate daily total count value of the user.

At Step 4098, an aggregate daily total count value for a first time period may be determined. In some embodiments, the aggregate daily total count value may be determined by averaging a count total for each day in the first time period. In other embodiments, the aggregate daily total count value may be determined by identifying a median count total for each day in the first time period.

At Step 4100, a target daily count goal for a user for a second time period may be determined based on the determined aggregate daily total count value for the first time period.

In some embodiments, the target daily count total for the new time period (e.g., the next week), may be determined in part from a comparison of the user to a population of other users with similar count scores. In some embodiments, the population may have scores within about +/−10%, alternatively about +/−15%, alternatively about +/−20%. In some embodiments, the target daily count goal for the user for the second time period is determined based on a comparison of the determined aggregate daily total count value for the first time period to a population of users having an aggregate daily total count value within a threshold of the determined aggregate daily total count value of the user. In some embodiments, the comparison may be to users in a similar demographic.

In some embodiments, the first time period is a first week and the second time period is a second week. The first and second time periods may be consecutive or non-consecutive. The second time period may occur after the first time period.

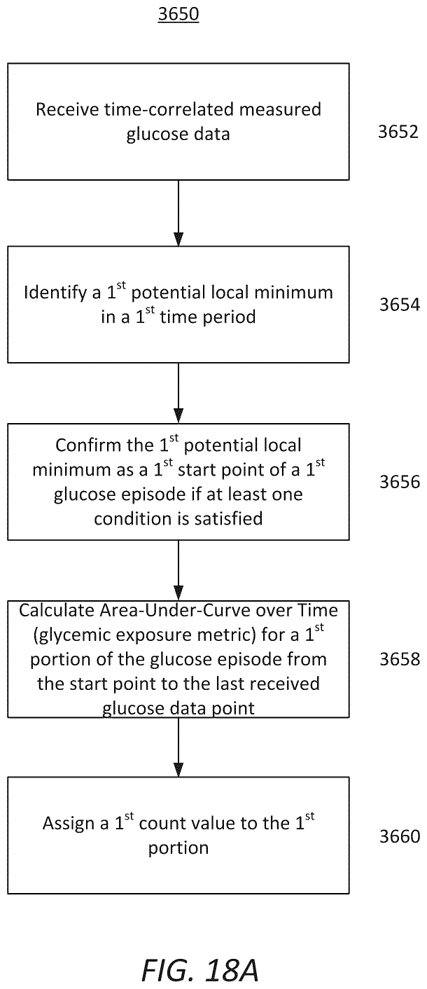

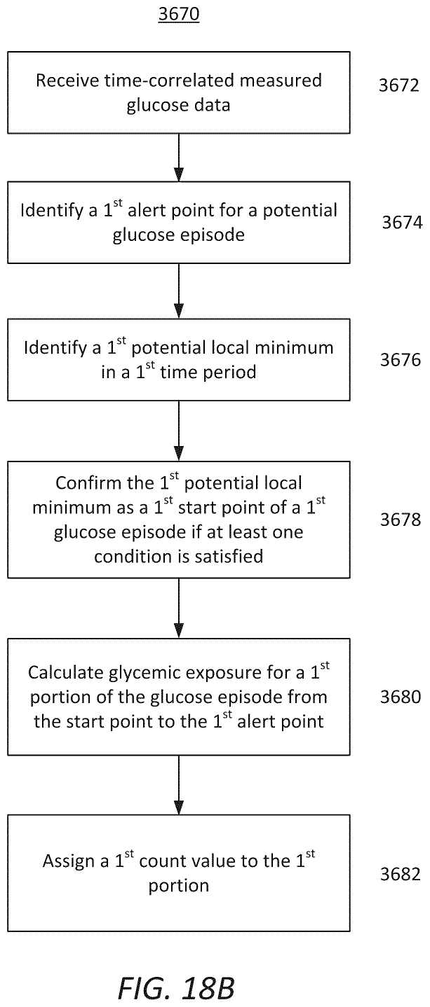

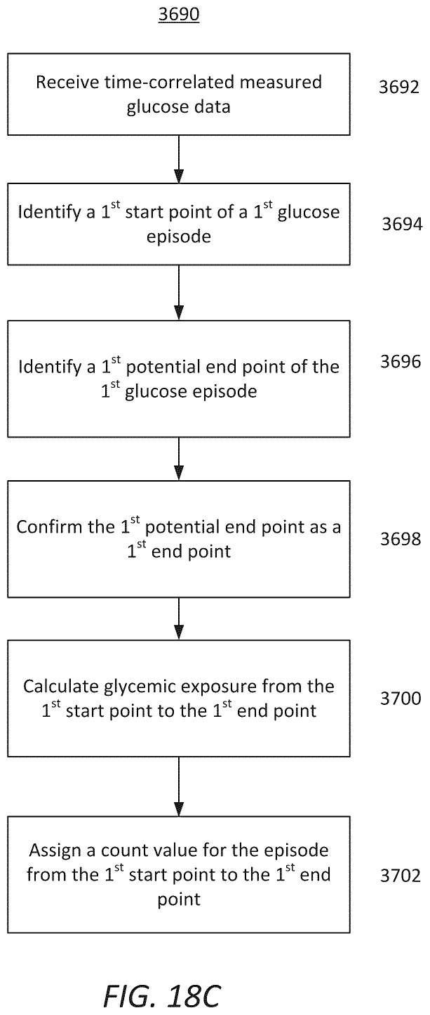

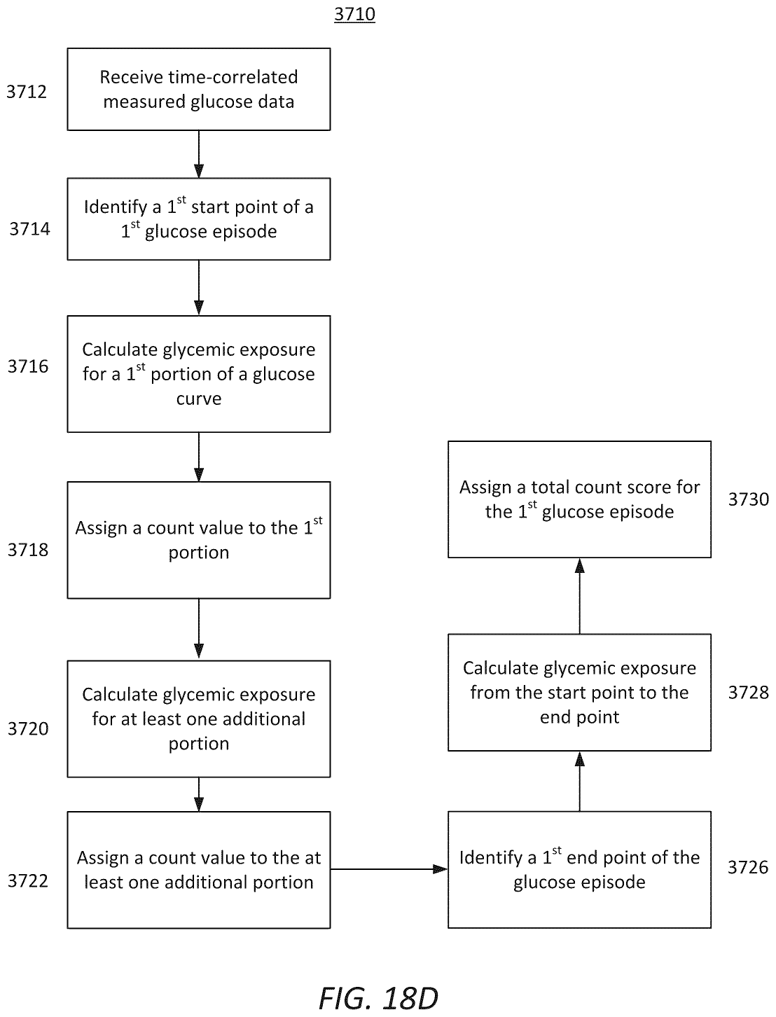

Turning next to FIG. 18A, a flow diagram depicts an example embodiment of a method 3650 for assessing the daily count performance of an individual. In some methods, the daily counts may be determined in real time, as a peak or spike is occurring. At Step 3652, time-correlated measured glucose data is received. According to some embodiments, the data can be received by a reader 120, local computer system 170, trusted computer system 180, or any computing device used in conjunction with an analyte monitoring system.

At Step 3654, the received data is analyzed, and a first potential local minimum is identified in a first time period. The first time period may include a beginning data point and a last received glucose data point (or a current glucose data point). In some embodiments, the beginning glucose data point may have a timestamp that is between about 60 to about 90 minutes, alternatively between about 60 to about 80 minutes, alternatively between about 60 to about 120 minutes, alternatively about 60 minutes, alternatively about 65 minutes, alternatively about 70 minutes, alternatively about 75 minutes, alternatively about 80 minutes, alternatively about 85 minutes, alternatively about 90 minutes, alternatively about 120 minutes before a timestamp of the last received glucose data point. In other embodiments, the beginning data point may be an end point of a previous adjacent glucose spike or episode or the end point of the glucose spike that previously occurred closest in time to the current glucose spike.

At Step 3656, the first potential local minimum may be confirmed as a first start point (see, e.g., 3762 of FIG. 19A) of the first glucose spike or episode if at least one condition is satisfied. In some embodiments, the first potential local minimum may be confirmed as the first start point if a rate of change between the first potential local minimum and a previous point within the first time period is above a rate of change threshold value. The previous point within the first time period that is being compared may be a point with a timestamp within the last about 20 minutes of the first potential local minimum. In some embodiments, the previous point may be the previous adjacent point to the first potential local minimum (i.e., the point received before the first potential local minimum). In other embodiments, the previous point may be the second to last point before the first potential local minimum or the penultimate point. In some embodiments, the first potential local minimum may be confirmed as the first start point if a difference in a level between the first potential local minimum and an alert point is above a threshold.

At Step 3658, a glycemic exposure metric may be calculated for a first portion of the glucose episode starting from the start point to the last received glucose data point. The glycemic or glucose exposure metric may be an area-under-the-curve over time, or an integrated area-under-the-curve over time.

At Step 3660, a first count value may be assigned to the first portion of the glucose episode based on the calculated glycemic exposure metric. In some embodiments, the count value may be based on a comparison to a distribution of the glycemic exposure metric determined from a predetermined population.

Turning next to FIG. 18B, a flow diagram depicts an example embodiment of a method 3670 for assessing the daily count performance of an individual that includes determining an alert point for a glucose episode. In some methods, the daily counts may be determined in real time, as a peak, spike, or episode is occurring. At Step 3672, time-correlated measured glucose data is received. According to some embodiments, the data can be received by a reader 120, local computer system 170, trusted computer system 180, or any computing device used in conjunction with an analyte monitoring system.

In an optional step, at Step 3674, a first alert point (see, e.g., 3766 of FIG. 19A) for a potential glucose episode may be identified. A last received glucose data point may be identified as a first alert point in a first time period if the last received glucose data point satisfies at least one alert condition. In some embodiments, the at least one alert condition includes confirming that a calculated rate of change between the first potential alert point and a previous point within about 20 minutes of the first potential alert point is above an alert rate of change threshold. In some embodiments, the at least one alert condition includes confirming that a calculated rate of change between the first potential alert point and a second previous data point (i.e., the data point received before the previous data point) is above an alert rate of change threshold. In some embodiments, the at least one alert condition includes confirming that a difference between the first potential alert point and the first potential local minimum is above a local minimum alert threshold. In some embodiments, the at least one alert condition includes confirming that a calculated integrated area under the curve from the first potential local minimum to the first potential alert point corresponds to a count value above a threshold count value. In some embodiments, an alert point is identified if the last received glucose data counts satisfies a single alert condition. In other embodiments, an alert point is identified if the last received glucose data counts satisfies multiple alert conditions.

In an optional step, in some embodiments, a glucose baseline is calculated for a day. In some embodiments, the glucose baseline may be calculated based on the first data point of each new calendar day. The glucose baseline may be defined as the level at certain percentile of non-clipped glucose values from the previous 24 hours.

At Step 3676, the received data is analyzed and a first potential local minimum is identified in a first time period. The first time period may include a beginning data point and a last received glucose data point (or a current glucose data point). In some embodiments, the first time period may include a beginning data point up to the first alert point. In some embodiments, the beginning glucose data point may have a timestamp that is between about 60 to about 90 minutes, alternatively between about 60 to about 80 minutes, alternatively between about 60 to about 120 minutes, alternatively about 60 minutes, alternatively about 65 minutes, alternatively about 70 minutes, alternatively about 75 minutes, alternatively about 80 minutes, alternatively about 85 minutes, alternatively about 90 minutes, alternatively about 120 minutes before a timestamp of the last received glucose data point or the first alert point. In other embodiments, the beginning data point may be an end point of a previous adjacent glucose episode or the end point of the glucose episode that previously occurred closest in time to the current glucose episode.

At Step 3678, the first potential local minimum may be confirmed as a first start point (see, e.g., 3762 of FIG. 19A) of the first glucose episode if at least one condition is satisfied. In some embodiments, the first potential local minimum may be confirmed as the first start point if a rate of change between the first potential local minimum and a previous point within the first time period is above a rate of change threshold value. The previous point within the first time period that is being compared may be a point with a timestamp within the last about 20 minutes of the first potential local minimum. In some embodiments, the previous point may be the previous adjacent point to the first potential local minimum or the penultimate point before the first potential local minimum. In other embodiments, the previous point may be the second to last point before the first potential local minimum. In some embodiments, the at least one condition that must be satisfied to confirm the first potential local minimum as the first start point is that the first potential local minimum is a local minimum. In some embodiments, the first potential local minimum may be confirmed as the first start point if the glucose rise or rate of change from the first potential local minimum to the first alert point is above a threshold value. In some embodiments, the first potential local minimum may be confirmed as the first start point if the glucose exposure from the first potential local minimum to the first alert point is above a threshold exposure value. In some embodiments, the first potential local minimum may be confirmed as the first start point if the glucose variability from the first potential local minimum to the first alert point is above a variability value.

At Step 3680, a glycemic exposure metric may be calculated for a first portion of the glucose episode starting from the start point to the last received glucose data point. The glycemic exposure metric may be an area-under-the-curve over time, or an integrated area-under-the-curve over time.

At Step 3682, a first count value may be assigned to the first portion of the glucose episode based on the calculated glycemic exposure metric. In some embodiments, the count value may be based on a comparison to a distribution of the glucose exposure metric determined from a predetermined population.

Optionally, in some embodiments, after the first start point and the first alert point are confirmed, the wellness application may output a notification and/or alert. The notification and/or alert may inform the user that they are currently spiking or in a glucose spike or episode. Optionally, a glycemic exposure metric may be calculated from the first start point to the first alert point, and a count value may be assigned to this glycemic metric. In some embodiments, the notification and/or alert may include the count value of the episode calculated from the first start point to the first alert point.

Optionally, in some embodiments, after the first potential local minimum is confirmed as the first start point, a method may include the step of identifying a nearest additional local minimum occurring before the first start point. The optional method may further include the step of determining if the nearest additional local minimum satisfies at least one condition. As stated previously with regard to step 3678, the nearest additional local minimum may be confirmed as the first start point if a rate of change between the nearest additional local minimum and a previous point is above the rate of change threshold value. The previous point that is being compared may be a point with a timestamp within the last about 20 minutes of the nearest additional local minimum. In some embodiments, the previous point may be the previous adjacent point to the nearest additional local minimum or the penultimate point before the nearest additional local minimum. In other embodiments, the previous point may be the second to last point before the first potential local minimum.

Turning next to FIG. 18C, a flow diagram depicts an example embodiment of a method 3690 for assessing the daily count performance of an individual that includes determining an end point for a glucose episode. In some methods, the daily counts may be determined in real time, as a peak, spike, or episode is occurring. At Step 3692, time-correlated measured glucose data is received. According to some embodiments, the data can be received by a reader 120, local computer system 170, trusted computer system 180, or any computing device used in conjunction with an analyte monitoring system.

At Step 3694, a first start point (see, e.g., 3762 of FIG. 19A) of a first glucose episode is identified.

At Step 3696, a first potential end point of the first glucose episode is identified. At Step 3698, the first potential end point is confirmed as the first end point (see, e.g., 3764 of FIG. 19A) if the first potential end point satisfies at least one end point condition.

In some embodiments, the first potential end point is confirmed as the first end point if a difference between a glucose level of the first start point of the first glucose episode and a glucose level of the first potential end point is below a threshold difference. In other embodiments, the first potential end point is confirmed as the first end point if the first potential end point is a local minimum as compared to a previous adjacent data point. In some embodiments, the first potential end point is confirmed as the first end point if a calculated integrated area under the curve over time of a portion of the graph from the start point to the first potential end point is less than a minimum episode point threshold score. In other embodiments, an episode duration from the start point to a potential end point must be above a minimal length of time. In other embodiments, a glucose difference between the glucose level at the end point and the glucose level at the start point may be compared. In some embodiments, a potential end point is identified as the end point if the potential end point satisfies a single end point condition. In other embodiments, a potential end point is identified as the end point if the potential end point satisfies multiple end point conditions.

At Step 3700, a glycemic exposure metric is calculated from the first start point to the first end point. In some embodiments, the glycemic exposure metric may be an area-under-the-curve over time, or an integrated area-under-the-curve over time.

At Step 3702, a count value may be assigned to the episode in the glucose curve from the first start point to the first end point based on the calculated glycemic exposure metric. In some embodiments, the count value may be based on a comparison to a distribution of the glucose exposure metric determined from a predetermined population.

Turning next to FIG. 18D, a flow diagram depicts an example embodiment of a method 3710 for assessing the daily count performance of an individual that includes calculating a count score for a glucose episode from a start point to an end point. In some methods, the daily counts may be determined in real time, as a peak, spike, or episode is occurring. At Step 3712, time-correlated measured glucose data is received. According to some embodiments, the data can be received by a reader 120, local computer system 170, trusted computer system 180, or any computing device used in conjunction with an analyte monitoring system.

At Step 3714, a first start point (see, e.g., 3762 of FIG. 19A) of a first glucose episode is identified.

At Step 3716, a glycemic exposure metric may be calculated for a first portion of a glucose episode from the start point to a last received glucose data point in a first time period. In some embodiments, the glycemic exposure metric may be an area-under-the-curve over time, or an integrated area-under-the-curve over time. In some embodiments, the last received glucose data point may be an alert point. At Step 3718, a count value is assigned to the first portion. In some embodiments, the count value may be based on a comparison to a distribution of the glucose exposure metric determined from a predetermined population.

At Step 3720, a glycemic exposure metric may be calculated for at least one additional portion of the glucose episode from the start point to a last received glucose data point in at least one additional time period. In some embodiments, the glycemic exposure metric may be an area-under-the-curve over time, or an integrated area-under-the-curve over time. At Step 3722, a count value is assigned to the at least one additional portion of the glucose episode from the start point to the last received glucose data point in the at least one additional portion. In some embodiments, the last received glucose data point may be the next data point after the last received data point in the first portion. In some embodiments, the count value may be based on a comparison to a distribution of the glucose exposure metric determined from a predetermined population.

At Step 3726, a first end point (see, e.g., 3764 of FIG. 19A) of the first glucose episode may be identified.

At Step 3728, a glycemic exposure metric may be calculated for the episode from the start point to the end point. In some embodiments, the glycemic exposure metric may be an area-under-the-curve over time, or an integrated area-under-the-curve over time. At Step 3730, a total count score may be assigned for the metric calculated from the start point to the end point. In some embodiments, the count value may be based on a comparison to a distribution of the glucose exposure metric determined from a predetermined population.

For example, after an episode is detected, the first glucose count may be computed after about 15 minutes after the episode started. The next computation may occur about 30 minutes after the episode. These periodic computations may continue until the end of the episode is detected. The user's total glucose counts may be updated accordingly after each computation.

Any of the methods described in this application that include steps of assigning counts described herein may utilize a conversion formula to linearize the daily exposure values to a daily count range is established. According to some embodiments, for example, the daily exposure values (e.g., integrated area under the curve over time) of a main portion of a population distribution (e.g., 0 to 90th percentile, alternatively 0 to 80th percentile, alternatively 0 to 75th percentile) can be linearized to a range of daily glucose counts (e.g., between 0 and 100 counts). In some embodiments, for example, the glucose counts can comprise a continuous system from 0 to 100 counts, with intervals of one. In addition, according to some embodiments, for daily exposure values above the 90th percentile, a count value can still be calculated using the same conversion formula—that is, with a count value above 100—but can be capped at a predetermined maximum count value (e.g., 500 counts). This would allow the count system to be used by pre-diabetic and diabetic users, or individuals with high glycemic exposure.

Any of the methods described in this application that calculate or determine an area under the curve, or an area under the curve divided by time, may be determined with respect to a non-zero base value. In some embodiments, the area under the curve, or an area under the curve divided by time may be calculated from a base value of between about 50 mg/dL to about 100 mg/dL, alternatively between about 60 mg/dL to about 100 mg/dL, alternatively between about 60 mg/dL to about 90 mg/dL, alternatively between about 70 mg/dL to about 90 mg/dL, alternatively about 60 mg/dL, alternatively about 65 mg/dL, alternatively about 70 mg/dL, alternatively about 75 mg/dL, alternatively about 80 mg/dL, alternatively about 85 mg/dL, alternatively about 90 mg/dL, alternatively about 95 mg/dL, alternatively about 100 mg/dL. The calculation may be done using a non-zero base value because glycemic exposure below a certain threshold (e.g., about 70 mg/dL) is healthy, and the glucose wellness application does not want a user to lower their glucose levels into a hypoglycemic range.

For episodes that are in progress at a specified time that bridges two time periods (e.g., midnight), the algorithm in the wellness application may automatically terminate the episode at the last data point before the end of the previous time period (e.g., 11:59 pm), and assign the counts accumulated in the episode before the specified time to the previous time period. For the remainder of the episode that was in progress, the algorithm in the wellness application may set the next data point after the specified time as the start of a new episode for the new time period (e.g., the day starting at 12:00 am), and continue to calculate glycemic exposure and assign counts until the end of the episode is detected, thus the counts from a single episode may be split between the previous time period (e.g., day 1) and new time period (e.g., day 2). In another example, the two time periods may be time of day periods within a single day, e.g., afternoon and evening, and the specified time that bridges the two time periods may be 4 pm.

If data is missing in an identified episode as it is progressing, the algorithm may end the episode and the algorithm may calculate the counts associated with the episode. In some embodiments, the data may need to be missing for at least an hour, alternatively at least 30 minutes, in order for the algorithm to terminate a episode due to missing data.

### Live/Home Screens

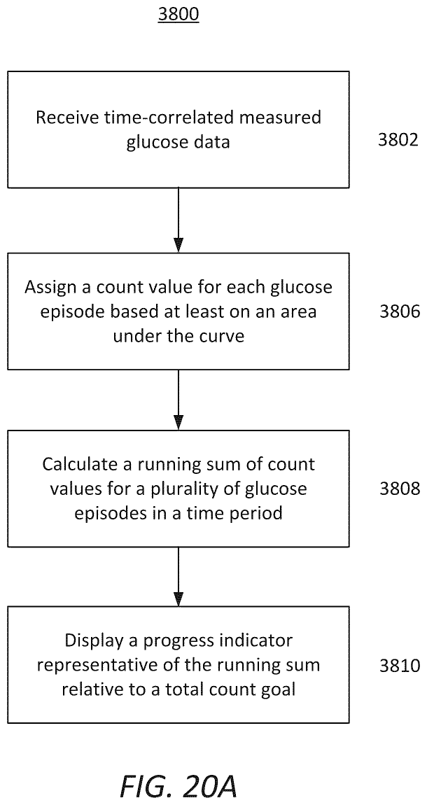

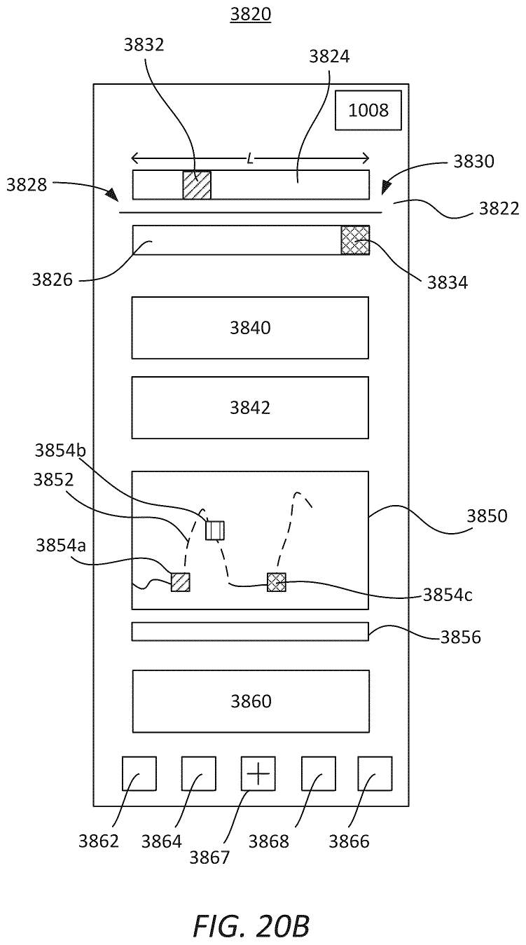

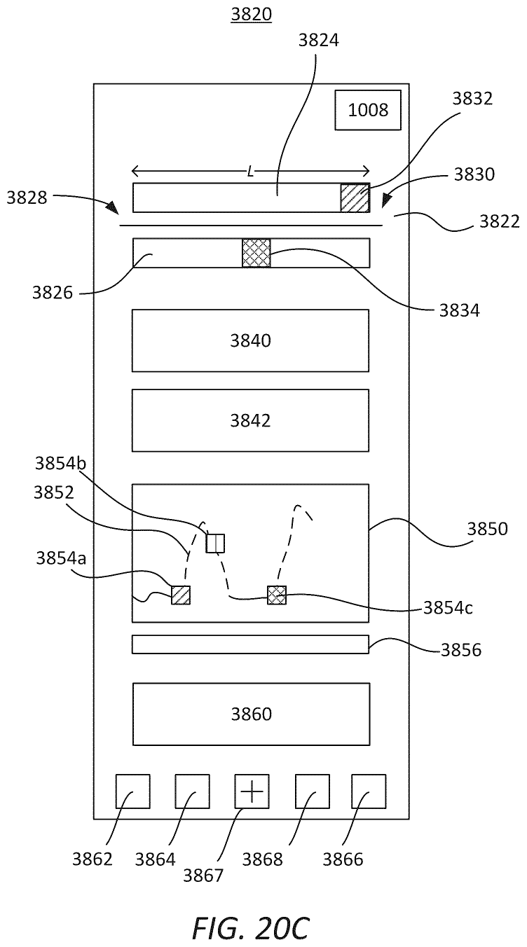

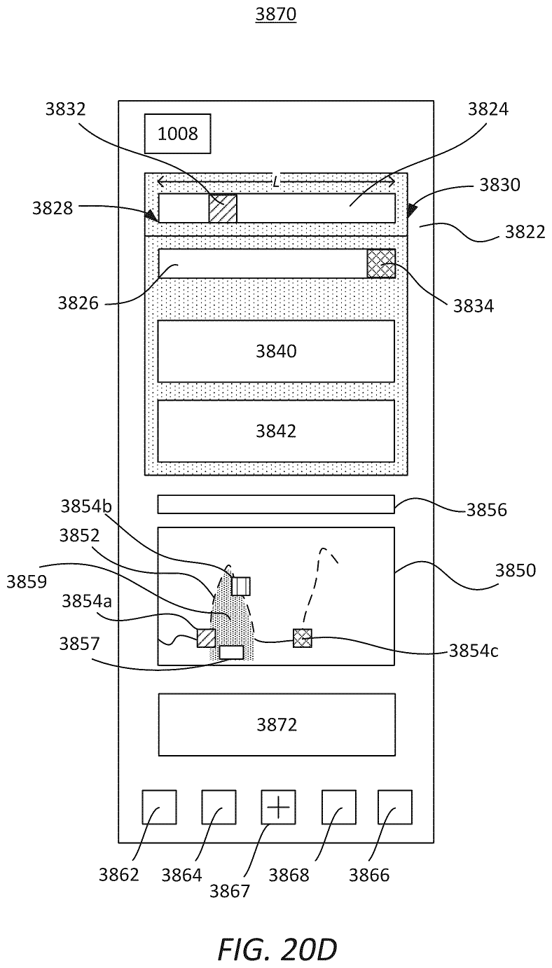

As seen in FIGS. 20B-20C, the glucose wellness application may include a live or home screen 3820 displaying a progress indicator 3822, a message 3840 regarding the user's current glucose trend status, a recommendation 3842, an event summary for the day 3850 that includes a graph 3852 and may also include a plurality of icons 3854a-3854c, options 3856 for displaying a different day or time frame, and a recommendations section 3860. Links may also be displayed to enable the user to quickly view GUIs related to today's live screen 3862, their journey 3864, which may link to GUIS including various summaries or reports of data in past time periods, and discover 3868, which may link to GUIS including various content that may explain concepts, make recommendations, or otherwise provide helpful information to assist the user in improving their glycemic control.

As seen in FIG. 20C, the glucose wellness application may include a live or home screen 3870 displaying a progress indicator 3822, a message 3840 regarding the user's current glucose trend status, a recommendation 3842, an event summary for the day 3850 that includes a graph 3852 and may also include a plurality of icons 3854a-3854c, options 3856 for displaying a different day or time frame, and a logged events section 3872, which may correspond with the plurality of icons 3854a-3854c appearing in the graph 3852. The glucose spikes or excursions in the graph 3852 or trace may also include a label 3857 of the number of counts assigned to the glucose spike. In some embodiments, the area under the curve of the glucose spike or excursion that is used to determine the number of corresponding counts may also be shaded 3859. Links may also be displayed to enable the user to quickly view GUIs related to today's live screen 3862, their journey or plan 3864, which may link to GUIs including various summaries or reports of data in past time periods, log 3867, which may include an editable log of events and entries, and discover 3868, which may link to GUIS including various content that may explain concepts, make recommendations, or otherwise provide helpful information to assist the user in improving their glycemic control.

### Progress Indicator for Daily Counts

The wellness application may calculate a running sum of counts earned throughout a time period for a peak (excursion) or multiple peaks (excursions). In some embodiments, the calculated running sum may be displayed associated with a user's target count goal so that the user may easily assess how much of their budget has been spent and how much of their budget remains for the day. Turning next to FIG. 20A, a flow diagram depicts an example embodiment of a method 3800 for assessing the daily count performance of an individual and displaying a progress indicator representative of a running glucose counts sum relative to a target count goal. In some methods, the daily counts may be determined in real time, as a peak or spike is occurring. At Step 3802, time-correlated measured glucose data is received. According to some embodiments, the data can be received by a reader 120, local computer system 170, trusted computer system 180, or any computing device used in conjunction with an analyte monitoring system.

At Step 3806, a count value may be assigned for each glucose episode based at least on an area under the curve of the each glucose episode in a dataset of time-correlated glucose data. In some embodiments, the count value is assigned based on a comparison to a distribution of the glucose metric determined from a predetermined population. In some embodiments, the count may be assigned based on an area-under-the-curve over time, or an integrated area-under-the-curve over time. In some methods, the counts may be assigned in real time, as a peak or spike or episode is occurring. In some embodiments, the count may be determined retrospectively after a peak or spike or episode has ended or at the end of a period of time, e.g., about 1 hour, about 2 hours, about 4 hours, about 6 hours, about 12 hours, about 24 hours.

At Step 3808, a running sum of the count values for a plurality of glucose episodes in a time period may be calculated. In some embodiments, the time period may be one day, one week, or one month.

At Step 3810, a progress indicator representative of the running sum relative to a target count goal for the time period may be displayed.

In some embodiments, as seen in GUI 3820 of FIG. 20B, the progress indicator 3822 may be a fraction having the running sum of count values in a numerator 3824 and the target count goal in a denominator 3826. The display of the fraction has a first end 3828, a second end 3830, and a length L.

In some embodiments, where the running sum of count values is less than or equal to the target count goal, a numerical value of the running sum of count values 3832 may be displayed at a position along the length L of the numerator 3824 that is proportional to [the running sum of count values]/[the target count goal]. For instance, if the target count goal is 60 and the running sum of count values is 20, the “20” would be located approximately ⅓ of the length from the first end 3828. If the target count goal is 60 and the running sum of count values is 30, the “20” would be located approximately ½ of the length L of the numerator 3824. The numerical value of the target count goal may be located at the second end 3830 of the denominator.

In some embodiments, as seen in FIG. 20C, where the running sum of count values is greater than the target count goal, a numerical value of the target count goal 3834 is displayed at a position along the length L of the denominator that is proportional to [the target count goal]/[the running sum of count values]. The numerical value of the running sum of count values may be located at the second end 3830 of the numerator. For instance, if the target count goal is 60 and the running sum of count values is 80, the “60” would be located approximately ¾ of the length from the first end 3828. For instance, if the target count goal is 60 and the running sum of count values is 100, the “60” would be located approximately ⅗ of the length from the first end 3828.

In some embodiments, if the sensor is warming up, expired, or if there is a sensor error, then the numerator may be displayed as a dashed line “−” instead of a numerical value.

### Count Trend

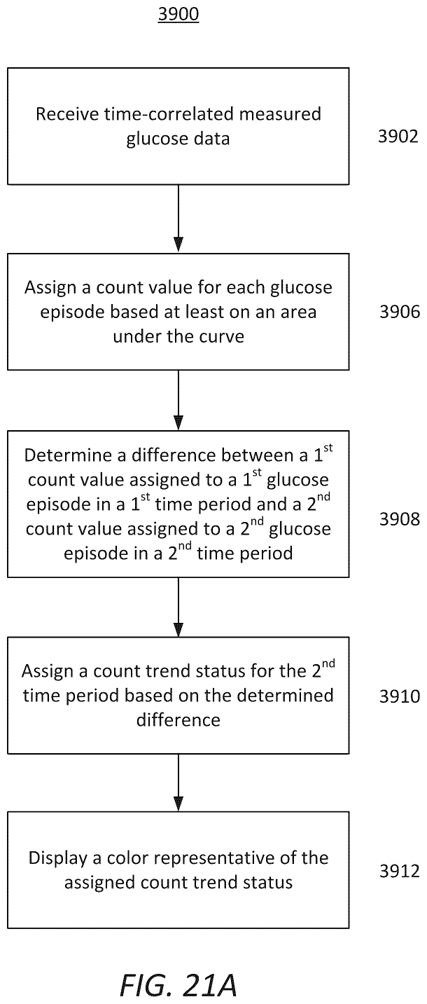

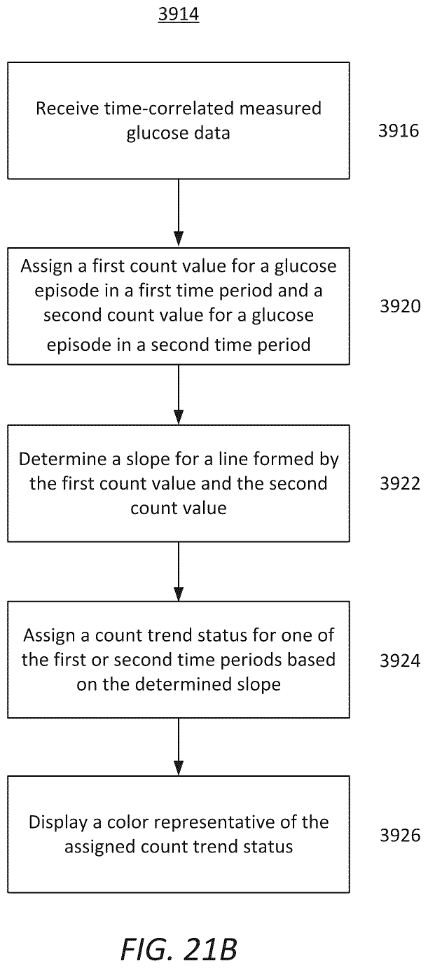

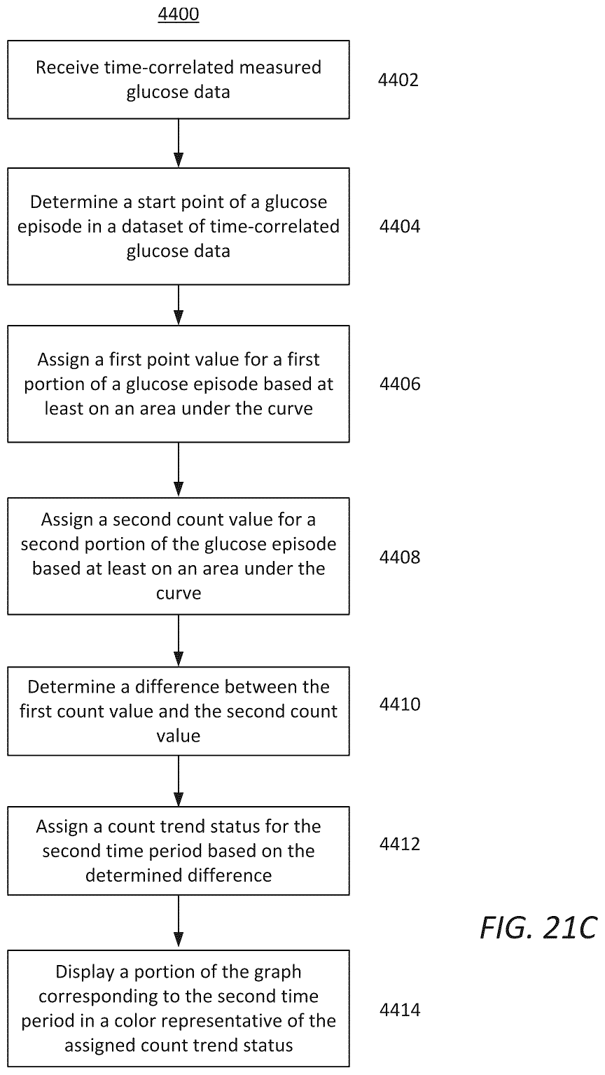

In some embodiments, the wellness application may display a color or graphic indicative of whether the user is currently experiencing a glucose spike or not. In some embodiments, a user's status may be determined by comparing a current count value to a previous count value. Turning next to FIG. 21A, a flow diagram depicts an example embodiment of a method 3900 for determining a count trend status and displaying a representation of the determined count trend status. At Step 3902, time-correlated measured glucose data is received. According to some embodiments, the data can be received by a reader 120, local computer system 170, trusted computer system 180, or any computing device used in conjunction with an analyte monitoring system.

At Step 3906, a count value is assigned based at least on an area under the curve of the each glucose episode in a dataset of time-correlated glucose data. In some embodiments, the count value is assigned based on a comparison to a distribution of areas under the curve determined from a predetermined population. In some embodiments, the count value may be assigned based on an area-under-the-curve over time, or an integrated area-under-the-curve over time.

At Step 3908, a difference between a first count value assigned to a first glucose episode in a first time period and a second count value assigned to a second glucose episode in a second time period is determined.

At Step 3910, a count trend status for the second time period is assigned based on the determined difference. In some embodiments, the count trend status may be assigned from a plurality of count trend status reflecting a balanced or spiked (rising, falling, or flat) state. If there is no difference in values and the count values are 0, a count trend status may be determined to be balanced, or not in a spike. If the count trend status for the second time period is less than the preceding count value from the first time period, then the count trend status may be determined to be declining in a spike. If the count trend status for the second time period is greater than the preceding count value from the first time period, than the count trend status may be determined to be rising in a spike. If there is no difference in values, and the count values are greater than 0, a count trend status may be determined to be flat during a spike.

At Step 3912, in an optional step, a color representative of the assigned count trend status may be displayed. The assigned color may be displayed in many different ways.

In some embodiments, in addition to or in replace of the color, a trend arrow may be displayed that is representative of the assigned count trend status.

In some embodiments, a user's status may be determined by comparing a count value to a plurality of surrounding count values. Turning next to FIG. 21B, a flow diagram depicts an example embodiment of another method 3914 for determining a count trend status and displaying a representation of the determined count trend status. At Step 3916, time-correlated measured glucose data is received. According to some embodiments, the data can be received by a reader 120, local computer system 170, trusted computer system 180, or any computing device used in conjunction with an analyte monitoring system.

At Step 3920, a first count value for a glucose episode in a first time period and a second count value for a glucose episode in a second time period are assigned based at least on an area under the curve of the each glucose episode in a dataset of time-correlated glucose data. In some embodiments, the count value is assigned based on a comparison to a distribution of the glucose metric determined from a predetermined population. In some embodiments, the first and second count values may be assigned based on an area-under-the-curve over time, or an integrated area-under-the-curve over time.

At Step 3922, a slope for a line formed by the first count value and the second count value is determined.

At Step 3924, a count trend status for one of the plurality of time periods is assigned based on the determined slope. In some embodiments, if the slope is positive, then the assigned count trend status may be rising in spike. If the slope is negative, then the assigned count trend status may be declining in spike. If the slope is constant or substantially constant, then the assigned count trend status may be flat in spike. In some embodiments, the count trend status may be assigned to a latest time period of the plurality of time periods.

At Step 3926, in an optional step, a color representative of the assigned count trend status may be displayed. The assigned color may be displayed in many different ways.

In some embodiments, in addition to or in replace of the color, a trend arrow may be displayed that is representative of the assigned count trend status.

In some embodiments, as seen in FIG. 19B, a GUI 3780 may be colored to reflect a current count trend status, or a count trend status of the latest time period. For example, a balanced status may be expressed as a first color, e.g., a cool color like blue. A declining in spike status may be expressed as a second color. A rising in spike status may be expressed as a third color. A flat during spike status may be expressed as a fourth color. The colors may be displayed in numerous parts of a GUI or different GUIs. For example, in some embodiments, the color reflecting the current status may be displayed as a background color. In one embodiment, a color reflecting a current status may be displayed as a first portion 3782 of a background of a GUI 3780. As a new count trend status is determined for a new time period, the corresponding color of the new count trend status may be displayed in the first portion 3782 of the background and the color of the count trend status for the previous time period may be displayed in a second portion 3784 of the GUI. In some embodiments, the first portion is a top portion and the second portion is a bottom portion of the GUI. Thus, a new color corresponding to the current status may be displayed in a top portion of a GUI and the colors corresponding to older time periods may be pushed downward on the GUI. In some embodiments, the GUI may have at least two portions with colors corresponding to the count trend status from at least two consecutive time periods being displayed, with the most recent time period depicted at a top of the GUI. In another embodiment, the GUI may have three portions and colors corresponding to the count trend status from three consecutive time periods may be displayed, with the most recent time period depicted at a top of the GUI. Color changes related to the count trend status may be reflected in any of the GUIs described herein. For example, a change in color of the background screen may occur for a portion of GUI 3820, seen in FIGS. 20B-20C. In some embodiments, a top portion of the GUI behind the progress indicator 3822 may reflect the current status trend and a second portion behind the text in 3840 and 3842 may reflect the previous status trend.

In some embodiments, a graph of glucose levels vs. time may include the colors determined from count trend statuses for different time periods. The colors may be displayed as the glucose trace. In other embodiments, the colors may be displayed in an area under the curve or in part of the area under the curve. For example, a color reflecting a rising in spike or declining in spike status may be displayed at a top portion of the area under the curve closest to the glucose trace. In some embodiments, the color may be displayed in about a top ⅓ portion of the area under the curve, alternatively a top ½ portion of the area under the curve. In some embodiments, the colors may be blended. In other embodiments, the colors are not blended.

As seen in FIG. 21C, a flow diagram shows an example embodiment of a method 4400 for determining a count trend status and displaying a representation of the determined count trend status. At Step 4402, time-correlated measured glucose data is received. According to some embodiments, the data can be received by a reader 120, local computer system 170, trusted computer system 180, or any computing device used in conjunction with an analyte monitoring system.

At Step 4404, a start point of a glucose episode in a dataset of time-correlated glucose data is determined.

At Step 4406, a first count value for a first portion of a glucose episode is assigned based at least on an area under the curve. In some embodiments, the first portion of the glucose episode begins at the start point and extends to a last received glucose data point in a first time period.

At Step 4408, a second count value for a second portion of the glucose episode is assigned based at least on an area under the curve. In some embodiments, the second portion of the glucose episode begins at the last received glucose data point in the first time period and extends to a last received glucose data point in a second time period, wherein the second time period is immediately after the first time period.

At Step 4410, a difference between the first count value and the second count value is determined.

At Step 4412, a count trend status for the second time period from a plurality of count trend statuses based on the determined difference.

At Step 4414, a graph of glucose data vs. time is displayed. In some embodiments, a portion of the graph corresponding to the second time period is displayed in a color representative of the assigned count trend status.

In optional steps, at least one additional count value for at least one additional portion of the glucose episode is assigned based at least on an area under the curve. The at least one additional portion of the graph may begin at the last received glucose data point in the at least one additional time period to the last received glucose data point in the at least one additional time period. The at least one additional time period may occur immediately after the second time period. In another optional step, a difference between the at least one additional count value and the second count value may be determined. A count trend status for the at least one additional time period may be assigned from a plurality of count trend statuses based on the determined difference. In another optional step, the graph of glucose data vs. time may be displayed, where a portion of the graph corresponding to the at least one additional time period is displayed in a color representative of the assigned count trend status for the at least one additional time period.

### Event Summary

An event summary for the day 3850 may include a graph 3852, which has a glucose trace. As seen in FIGS. 20B-20D, the glucose trace may have multiple colors, where a first color (see, e.g., solid line in graph 3852) is used for the portions of the graph that are steady or in balance, and a second color (see, e.g., dashed line in graph 3852) is used for the portions of the graph that in a spike. The graph 3852 may be and can include a plurality of icons 3854a-3854c, which may correspond to detected or logged events. In some embodiments, the graph may not include numerical values on the y-axis. In some embodiments, the GUI 3820 may support a tap and scroll feature, where the user could place a finger on the display and scroll horizontally along the graph and see the glucose value corresponding to the location of the user's finger.

Display options 3856 may appear below the graph 3850 (FIG. 20B) or above the graph 3850 (FIG. 20D). The display options 3856 may include different time ranges of the current day, different days, or a link to a calendar view where different days may be selected. The display options 3856 may include, the current day (e.g., “today”), the previous day (e.g., “yesterday”), list a specific date (e.g., “October 10th”), or a calendar icon that links to a calendar GUI. The display options 3856 may also include different time ranges to display, such as 4 hours, 8 hours, 12 hours, or 24 hours.

As seen in FIG. 20C, an event summary for the day 3850 may include a graph 3852 and may also include a plurality of icons 3854a-3854c, options 3856 for displaying a different day or time frame, and a logged events section 3872, which may correspond with the plurality of icons 3854a-3854c appearing in the graph 3852. The glucose spikes in the graph 3852 or trace may also include a label 3857 of the number of counts assigned to the glucose spike. In some embodiments, the area under the curve of the glucose spike that is used to determine the number of corresponding counts may also be shaded 3859.

### Logging

The Home or Live screen may also include a logged events section 3872. The logged events section 3872 may include a listing of events that the user has logged. The logged events may each include an icon corresponding to the type of logged event, e.g., fork and spoon for food and drinks consumed, or a running cartoon figure for exercise, the type of logged event, e.g., exercise, food and drinks, mood, or other event, a description of the event, and a time of the event.

The user may log an event multiple ways. In some embodiments, the user may log the event by selecting the log link 3867 or “+” at the bottom of various glucose wellness GUIs. In other embodiments, an icon with a question mark (“?”) may appear at the beginning of a spike if a user has not logged any event or activity in connection with the spike. If a user clicks, taps, or selects the icon, a logging GUI may appear in which the user may enter relevant information. After a user has logged an entry, either by tapping on the “?” icon or by otherwise logging the entry or activity, a icon depicting a symbol relevant to the entry may be displayed. For instance, a biker or runner icon may appear for logged exercise and a fork, plate, or food may appear for a logged meal or snack, and a bed may appear for logged sleep. In other embodiments, a notification or alert may be displayed on the lock screen of the display device. The notification or alert may appear a predetermined time period after a typical meal start time, which may have been previously entered by the user. In some embodiments, the notification may appear about 30 minutes after the scheduled meal time.

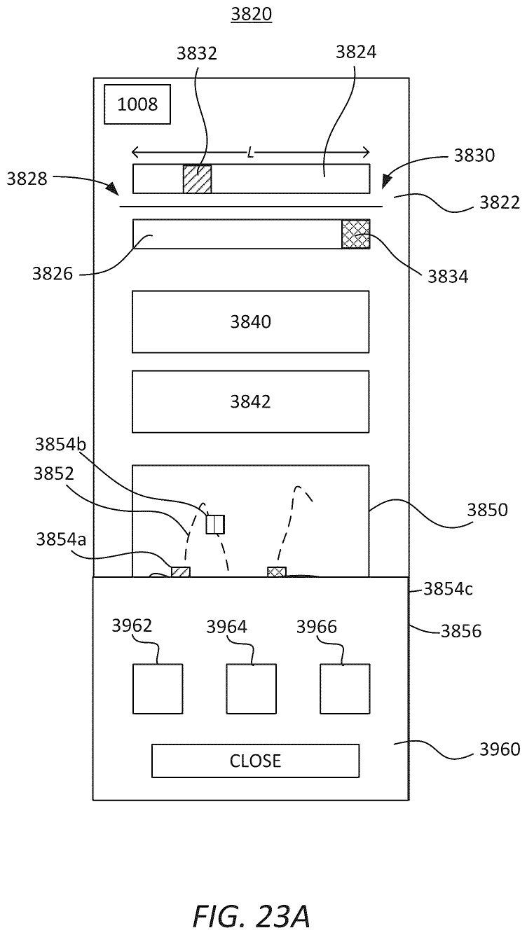

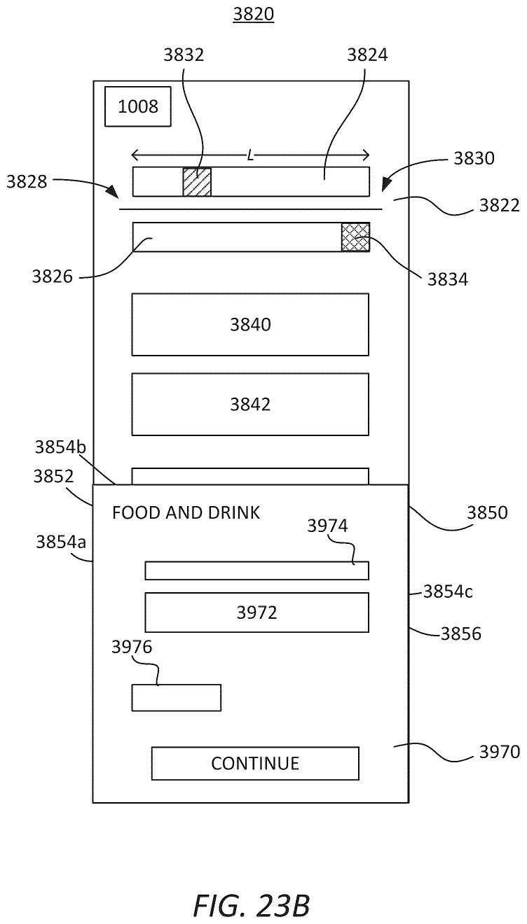

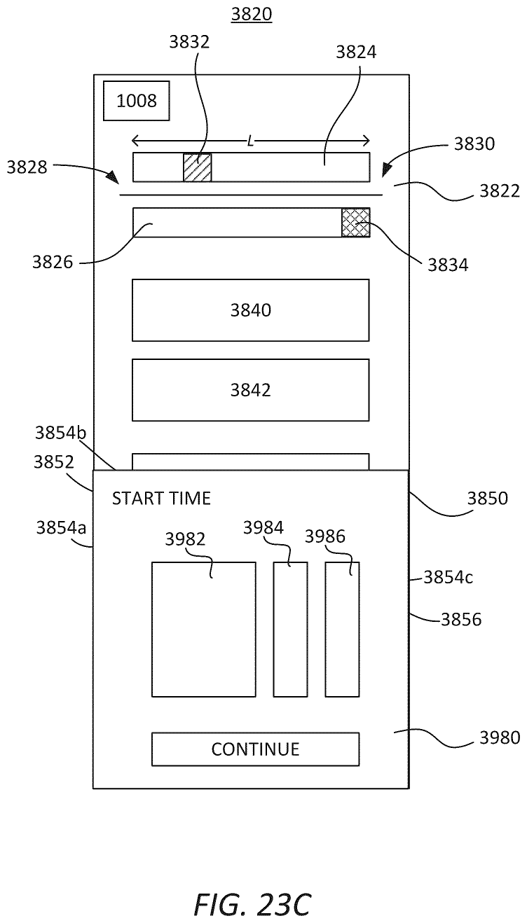

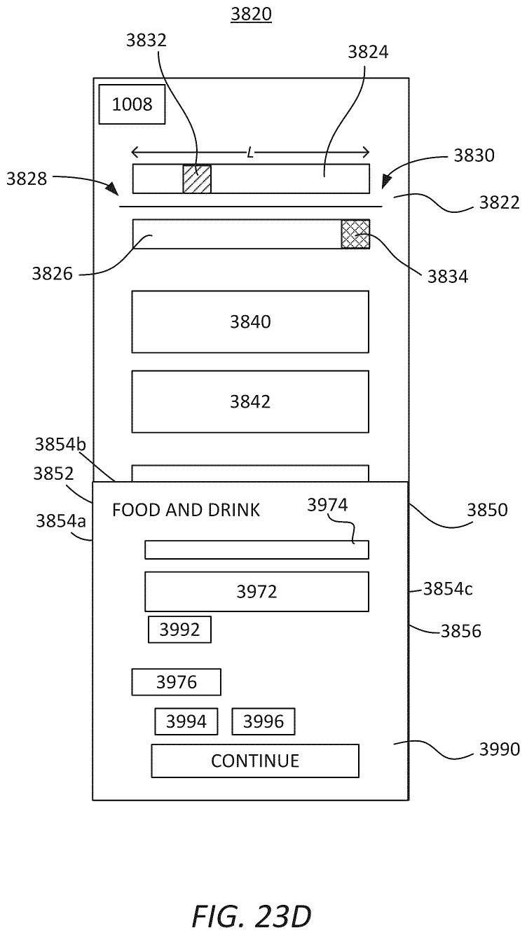

After a user selects “log” in a menu or home screen, a series of GUIs relating to logging may appear. As seen in FIG. 23A, a modal window 3960 or another GUI (not shown) may appear that includes links for logging food and drink 3962, exercise 3964, and other 3966.

If a user selects the food and drink link 3962, another modal window 3970 of GUI may appear, as seen in FIG. 23B. Modal window 3970 or GUI may include text 3974 asking the user what they ate or drank, along with a text box 3972 configured to receive a text description inputted by the user. In some embodiments, the modal window 3970 or GUI may also be configured to receive a picture or a tag of a previously entered food as input. Modal window or GUI may also include text 3976 prompting the user to enter the start time of the meal or snack and the date.

After the user enters the description of the food and/or drink, the user may select “continue” and, as seen in FIG. 23C, modal window 3980 or GUI may appear that includes an input for the time 3984, 3986 and date 3982 of the meal. The input may be a rolling wheel for each of the date 3982, hour 3984, and minute 3986 for the user to dial in the applicable information. Alternatively, the input may be a text box or a plurality of text boxes configured to receive the date and time associated with the meal, e.g., associated with the start of the meal. In some embodiments, the date and time stamp may be automatically populated. For instance, if the user clicks on a “?” icon, the date and time stamp may be automatically populated based on the detected beginning of the spike.

After the relevant information is entered, the user may select the button to set the date and time and modal window 3990 may appear, as seen in FIG. 23D. Similar to modal window 3960, modal window 3990 or GUI includes meal tag cloud 3972, date tag cloud 3994, and time tag cloud 3996. These tag clouds may be deleted or edited if the user wishes to edit any of the entries.

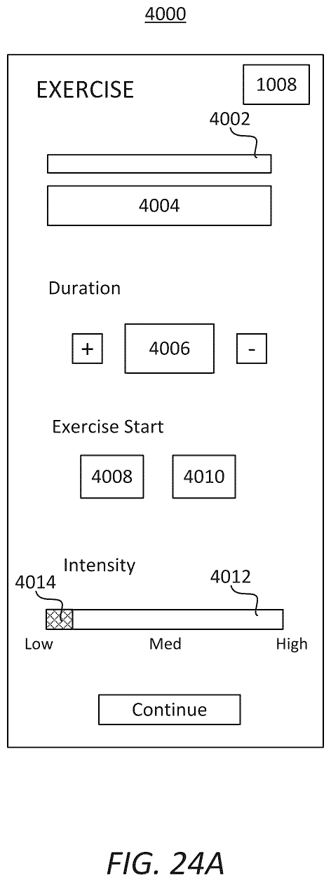

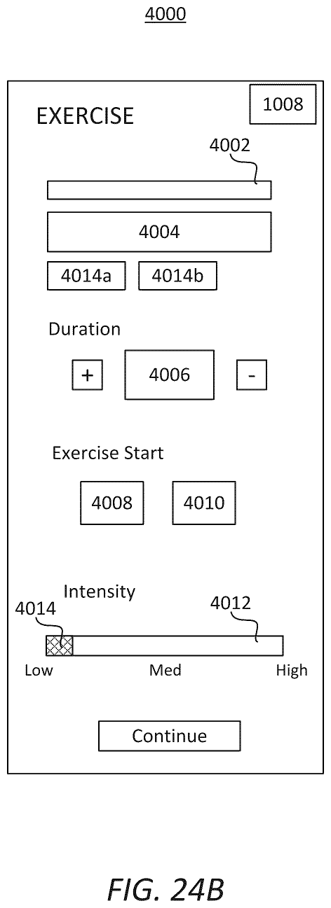

If a user selects the exercise link 3964, another modal window or GUI 4000 may appear. As seen in FIG. 24A, the modal window or GUI 4000 may include text 4002 asking the user what exercise they performed or participated in, along with a text box 4004 configured to receive a text description inputted by the user. In some embodiments, the modal window or GUI 4000 may also be configured to receive a picture or a tag of a previously entered exercise. After the relevant information is entered, as seen in FIG. 24B, exercise tag clouds 4014a-4014b may be displayed. These tag clouds may be deleted or edited if the user wishes to edit any of the entries.

The modal window or GUI may also include text prompting the user to enter the start time 4008 and date 4010 of the exercise. After the user enters the description of the exercise, the user may select “continue” and another modal window may appear that includes an input for the time and date of the exercise. The input may be a rolling wheel for each of the date, hour, and minute for the user to dial in the applicable information. Alternatively, the input may be a text box or a plurality of text boxes configured to receive the date and time associated with the exercise, e.g., associated with the start of the exercise. In some embodiments, the date and time stamp may be automatically populated. For instance, if the user clicks on a “?” icon, the date and time stamp may be automatically populated based on a detected beginning of a spike.

In some embodiments, a prompt may be displayed in the modal window or a new modal window or GUI 4000 that asks the user to input the intensity of the exercise. As seen in FIG. 24B, the input may be a sliding switch 4014 along a range of intensity 4012 that can be set from low or mild through medium to high or intense, or may prompt the user to select one of a series of numbers to indicate the intensity. Alternatively, the modal window may include a text box where the user can type in a description of the intensity of the exercise.

Unlogged events may be noted with a “?” icon on the graph. The user may select the log button and opt to remove the counts for this unlogged event.

Alternatively, for certain events in which the counts were not included in the daily total, the user may opt to add the counts to the daily total.

In some embodiments, the GUIs related to logging may include an option for the user to select to exclude counts attributed to a spike associated with the logged event from the user's budget. Thus, counts from the associated spike would not be added to the user's running sum for that day. For instance, the exercise logging GUI may include an option for the user to select to exclude counts attributed to a spike associated with the logged event from the user's budget.

In some embodiments, the counts attributed to a spike associated with a logged exercising event will not be included in the user's daily counts budget automatically.

In some embodiments, the wellness application may be linked to a health application or other sensors from which other data may be received that would indicate the user is exercising, such as increased heart rate or data from an accelerometer. The wellness application may optionally prompt the user to log an event if it detects that an exercise event is occurring or has occurred. In some embodiments, if the exercise event overlaps with the beginning of a spike or occurs within a predetermined time period of the start of the spike, the algorithm of the wellness application may automatically attribute the spike to an exercise event and exclude the associated counts from the daily count total. The predetermined time period may be between about 5 minutes and about 45 minutes, alternatively between about 5 minutes and about 30 minutes, alternatively between about 5 minutes and about 25 minutes, alternatively between about 5 minutes and about 20 minutes, alternatively between about 5 minutes and about 15 minutes, alternatively between about 5 minutes and about 10 minutes.

If a user selects the “other” link 3966, another modal window or GUI may appear. The modal window or GUI may include text asking the user what event they would like to add, along with a text box configured to receive a text description inputted by the user. For example, the user may wish to log stress, meditation, or sleep events. In some embodiments, the modal window or GUI may also be configured to receive a picture or a tag of a previously entered event. After the relevant information is entered, tag clouds may be displayed. These tag clouds may be deleted or edited if the user wishes to edit any of the entries.

The modal window may also include text prompting the user to enter the start time and date of the event. After the user enters the description of the event, the user may select “continue” and another modal window may appear that includes an input for the time and date of the event. The input may be a rolling wheel for each of the date, hour, and minute for the user to dial in the applicable information. Alternatively, the input may be a text box or a plurality of text boxes configured to receive the date and time associated with the event, e.g., associated with the start of the event. In some embodiments, the date and time stamp may be automatically populated. For instance, if the user clicks on a “?” icon, the date and time stamp may be automatically populated based on a detected beginning of a spike.

In another embodiment, a user may elect to exclude spike counts associated with non-food related events or activities, such as stress or exercise, from their daily total. While stress-related glycemic exposure may be deleterious, it is not necessarily amenable to improvement by modifying dietary intake. This would allow the user to concentrate entirely on their spikes related to food, or postprandial hyperglycemia.

In some embodiments, the user may track spike counts associated with a particular logged activity or event, such as stress or exercise, separately from their daily total counts related to food. Stress-related spikes may be similar in mechanism to high intensity exercise related spikes where cortisol and adrenaline are causing the liver to release glucose into the circulation. While stress-related glycemic exposure may be deleterious, it is not necessarily amenable to improvement by modifying dietary intake. Separately tracking non-food related spike counts may allow the user to employ separate strategies for their spikes related to food, or postprandial hyperglycemia, and their spikes related to non-food activities such as stress and/or exercise.

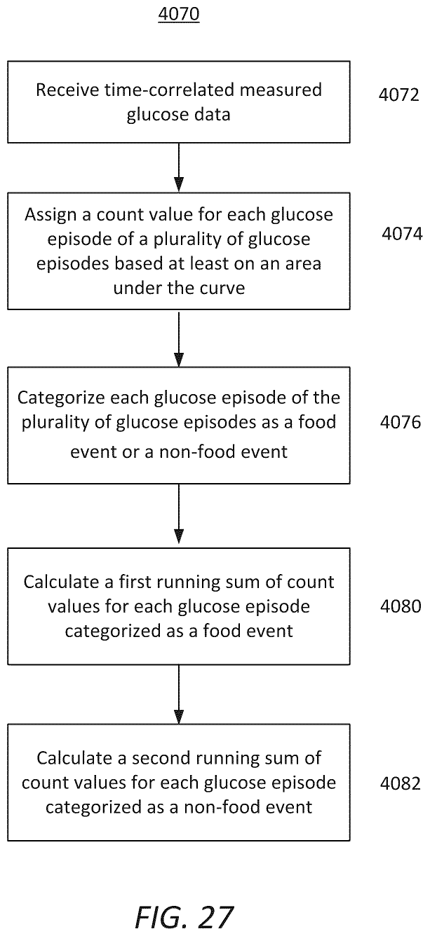

In one method, as seen in FIG. 27, a flow diagram depicts an example embodiment of another method 4070 for tracking counts related to food and non-food events. At Step 4072, time-correlated measured glucose data is received. According to some embodiments, the data can be received by a reader 120, local computer system 170, trusted computer system 180, or any computing device used in conjunction with an analyte monitoring system.

At Step 4074, a count value for each glucose episode of a plurality of glucose episodes in a dataset of time-correlated glucose data based at least on an area under the curve of the each glucose episode is assigned. In some embodiments, the count value assigned may be based on an area-under-the-curve over time, or an integrated area-under-the-curve over time. In some embodiments, the count value may be assigned based on a comparison to a distribution of the glucose metric determined from a predetermined population.

In Step 4076, each glucose episode of the plurality of glucose episodes may be categorized as a food event or a non-food event. As discussed elsewhere, such a determination or categorization may be made based on logged entries, tagging, flagging, or data received from other sensors or applications.

In Step 4080, in an optional step, a first running sum of count values for each glucose episode categorized as a food event may be calculated.

In Step 4082, in an optional step, a second running sum of count values may be calculated for each glucose episode categorized as a non-food event. For example, the glucose episode may have been caused by exercise and/or stress.

In a further optional step, each of the first and second running sums may be outputted to a display.

### Recommendations

The recommendations or tips section 3860 may include a plurality of recommendations aimed at assisting the user in reducing their glucose exposure and/or glucose levels. The recommendations may include tips to make the first meal of the day count by swapping juice for water (e.g., swapping orange juice for infused citrus water). The tips may also inform the user that protein and fat keep people fuller longer, and suggest adding protein (such as egg or avocado) to toast. The tips may also encourage the user to mix their coffee with cream or milk, but skip the sugar. The tips may also suggest having a smoothie for breakfast, and including protein powder, milk, fruit, and veggies in the smoothie. The tips may also encourage the user to hydrate in the morning with a glass of water. The tips may also encourage the user to practice yoga in the morning to get their body and mind ready for the day. The tips may also encourage the user to eat high fiber foods like porridge with fats like peanut butter to keep blood sugar spikes under control.

### Glucose Signatures

The glucose metrics and counts assigned based on the metrics can be used to assign a profile or signature to a user that highlights a time-of-day period on which the user can focus.

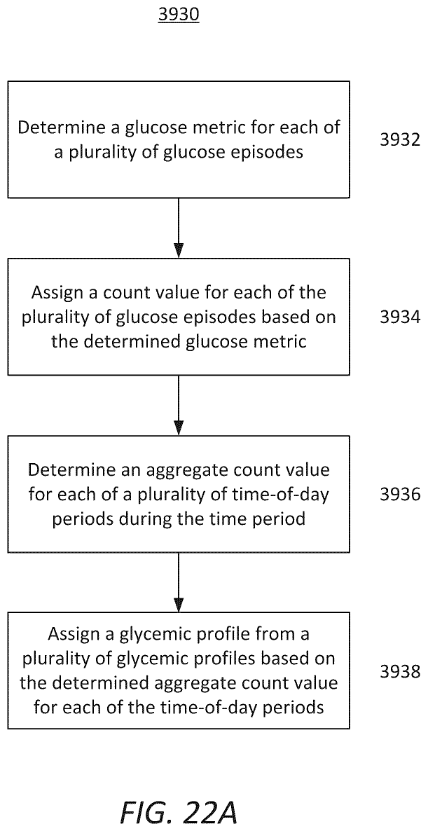

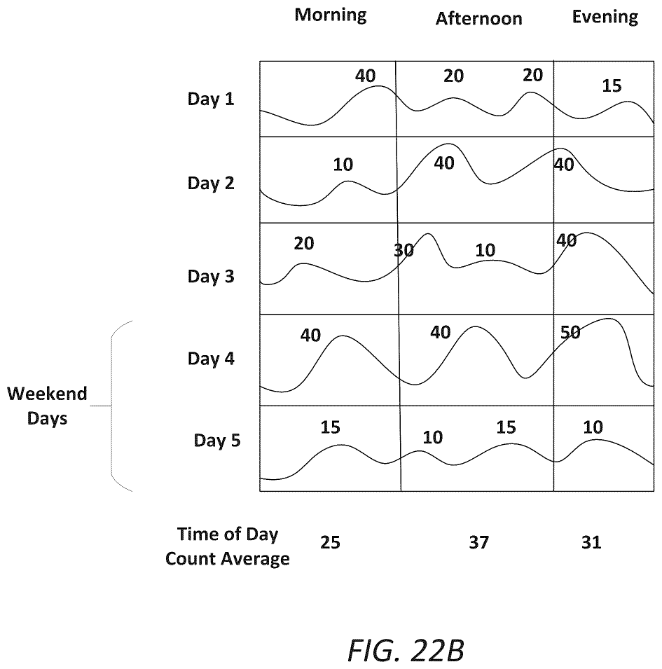

Turning next to FIGS. 22A-22B, a flow diagram depicts an example embodiment of a method 3930 for assigning a glucose signature or profile. At Step 3932, a glucose metric for each of a plurality of glucose episodes in a dataset of time correlated glucose data in a time period is determined. In some embodiments, the dataset includes a graph of glucose data vs. time over a time period may be determined. In some embodiments, the glucose metric may be a calculated integrated area under the curve for each of the plurality of glucose episodes.

At Step 3934, a count value for each glucose episode of the plurality of glucose episodes may be assigned. In some embodiments, the count value may be assigned based on a comparison of the determined glucose metric to a distribution of glucose metrics determined from a predetermined population.

At Step 3936, an aggregate count value for each of a plurality of time-of-day periods during the time period may be determined. In some embodiments, the plurality of time-of-day periods includes at least 3 time-of-day periods, alternatively 4 time-of-day periods. In some embodiments, the plurality of time-of-day periods comprises a morning period, an afternoon period, an evening period, and an overnight period.

In some embodiments, the aggregate count value for each of the plurality of time-of-day periods is determined by averaging a count total for each time-of-day period in the time period. In other embodiments, the aggregate count value for each of the plurality of time-of-day periods is determined by identifying a median count total for each time-of-day period in the time period. In other embodiments, the aggregate count value for each of the plurality of time-of-day periods is determined by determining a sum of a count total for each time-of-day period in the time period. The time period may be about 1 week, alternatively about 1 month.

At Step 3938, a glycemic or glucose profile from a plurality of glycemic profiles may be assigned based on the determined aggregate count value for each of the plurality of time-of-day periods.

In some embodiments, the glycemic profile may be assigned based on a determined time-of-day period having a highest aggregate count value. A first glycemic profile may be assigned if the determined aggregate count value is highest in a morning time-of-day period. A second glycemic profile may be assigned if the determined aggregate count value is highest in an afternoon time-of-day period (see, e.g., FIG. 22B). A third glycemic profile may be assigned if the determined aggregate count value is highest in an evening time-of-day period. A fourth glycemic profile may be assigned if the determined aggregate count value is highest in an overnight time-of-day period. A fifth glycemic profile may be assigned if the determined aggregate count values for at least two time-of-day periods are equal or substantially equal. Alternatively, the fifth glycemic profile may be assigned if the determined aggregate count values for all of the time-of-day periods do not vary by more than a threshold count difference. The fifth glycemic profile may highlight all time-of-day periods.

The method 3930 may optionally include outputting a recommendation based on the assigned glycemic profile. In some embodiments, the recommendation may be further based on at least one characteristic of a user selected from the group consisting of an age, a height, a weight, a BMI, a gender, a race, and an ethnicity. In some embodiments, the recommendation may be further based on at least one input logged by a user, the at least one input selected from the group consisting of food, stress, sleep, mood, and exercise. In some embodiments, the recommendation may be further based on a particular geographic location of a user.

### Daily Reports

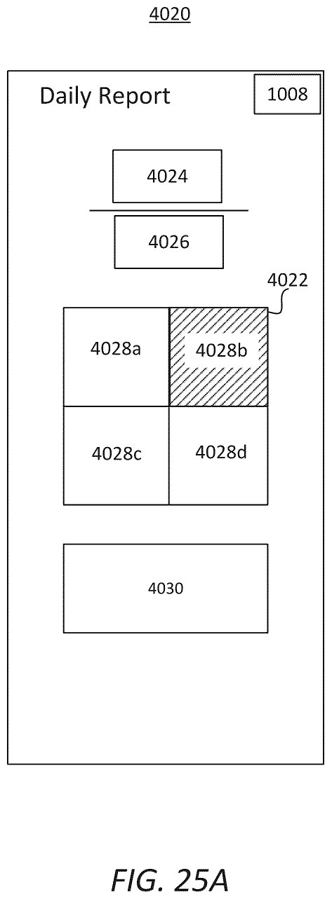

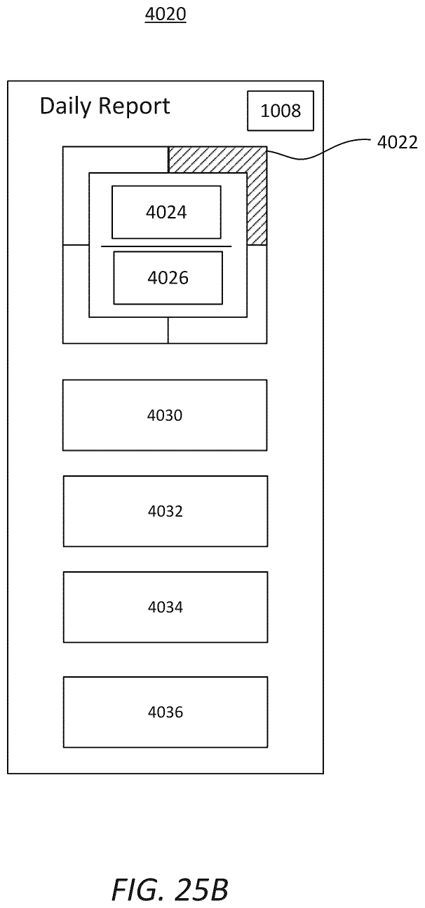

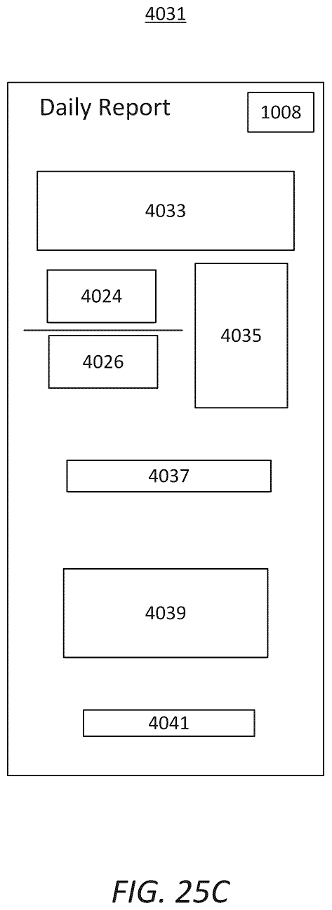

The glucose wellness application may prepare and display a daily report or briefing. A notification or an alert may be sent to inform the user that the daily report is available for viewing. As seen in FIG. 25A, the daily report may be displayed in a GUI 4020 that includes a graphical display 4022 of the previous day's count spend. The graphical display 4022 may have a portion corresponding to each time of day monitored. For example, the graphical display may have four portions 4028a-4028d corresponding to four times-of-day periods. The count total for each of the times of day may be displayed in each corresponding portion. The count total 4024 earned for the previous day may also be displayed. The count total 4024 for the day may be displayed relative to the daily total budget 4026. The count total 4024 for the day may be displayed as a numerator of a fraction and the daily total budget 4026 may be displayed as a denominator of the fraction.

In some embodiments, the graphical display 4022 may be a pie chart. In some embodiments, the graphical display 4022 may be a pie chart in the shape of an annular ring. In some embodiments, the graphical display 4022 may be a bar graph.

In some embodiments, the portion of the graph corresponding to the portion of the day with the highest count total may be highlighted in a different color (see, e.g., 4028b in FIG. 25A) than the rest of the portions of the graph.

In some embodiments, the fraction may be displayed in a center of the graphical display 4022, as seen in FIG. 25B.

The daily report GUI 4020 may also display recommendations 4030 for reducing the user's count score.

The daily report GUI 4020 or an additional GUI may also contain a statement 4032 regarding whether the user is in a streak or has multiple continuous days staying under counts budget.

The daily report GUI 4020 or an additional GUI may also contain a statement 4034 regarding whether the user exceeded their counts budget the following day and include recommendations to stay within their daily budget.

The daily report GUI 4020 or an additional GUI may also contain a list 4036 of untagged events, which the user can select to log events as described elsewhere in this application.

In some embodiments, as seen in FIG. 25C, the daily report may be displayed in a GUI 4031 that includes a message 4033 regarding the user's progress the previous day, a display of the counts 4024 earned the previous day relative to the counts goal 4026, a summary of the counts earned in various time of day periods 4035, a link for logging 4037, and a section 4039 prompting the user to provide input regarding their selected benefit or goal on which they are focusing. The message 4033 may include a summary or encouraging message. The message 4033 may state whether or not the user stayed within their counts budget or goal, or hit their benchmark. The message 4033 may also indicate in which time of day period the user accumulated the most counts. The count total 4024 earned for the previous day may be displayed. The count total 4024 for the day may be displayed relative to the daily total budget 4026. The count total 4024 for the day may be displayed as a numerator of a fraction and the daily total budget 4026 may be displayed as a denominator of the fraction. The summary of counts earned in various time of day periods 4035 may include a list of the time of day periods and the corresponding count total. In some embodiments, the time of day period in which the user accumulated the highest counts may be highlighted in a different color than the other time of day periods. The time of day periods may be, but are not restricted to, morning, afternoon, evening, and overnight.

The section 4039 prompting the user to provide input regarding their selected benefit on which they are focusing may include a prompt regarding the user's status with respect to the selected benefit over the last day or 24 hours. For example, if the user selected energy as their benefit focus, the section 4039 may include a prompt asking how was the user's energy over the last 24 hours. The section 4039 may include a range of selectable answers from which the user may choose relating to their energy level. In some embodiments, the selectable answers may include emojis and descriptions including, but not limited to, very bad, bad, neutral, good, and very good. In some embodiments, the selectable answers may include numerical values, e.g., 1, 2, 3, 4, and 5, and instructions that 1 refers to a lower level of energy and 5 refers to a higher level of energy. Alternatively, in some embodiments, the section 4039 may include a slidable button that can move from a range of very bad to very good, as indicated above, in which the user can move the button to indicate their appropriate level of energy. Similarly, where the user selected manage hunger as their benefit on which to focus, section 4039 may include a prompt asking how the user's hunger was over the last day or 24 hours. The selectable answers may range from very bad, bad, neutral, good, and very good. Similarly, where the user selected improve mood as their benefit on which to focus, section 4039 may include a prompt asking how the user's mood was over the last day or 24 hours. The selectable answers may include, but are not limited to, very bad, bad, neutral, good, and very good. Similarly, where the user selected better sleep as their benefit on which to focus, section 4039 may include a prompt asking how the user's sleep was over the last day or 24 hours. The selectable answers may include, but are not limited to, very bad, bad, neutral, good, and very good. Similarly, where the user selected stay focused as their benefit on which to focus, section 4039 may include a prompt asking how the user's focus was over the last day or 24 hours. The selectable answers may include, but are not limited to, very bad, bad, neutral, good, and very good. As described with respect to energy, for any of the benefits, section 4039 may include selectable answers that are numerical values, e.g., 1, 2, 3, 4, and 5. Alternatively, in some embodiments, the section 4039 may include a slidable button that can move from a range of very bad to very good, as indicated above, in which the user can move the button to indicate their appropriate response.

At the bottom of the GUI 4031, in some embodiments, a link to start their day 4041 may be included. If the user selects link 4041, the user may be taken to the home or live screen.

### Weekly Report

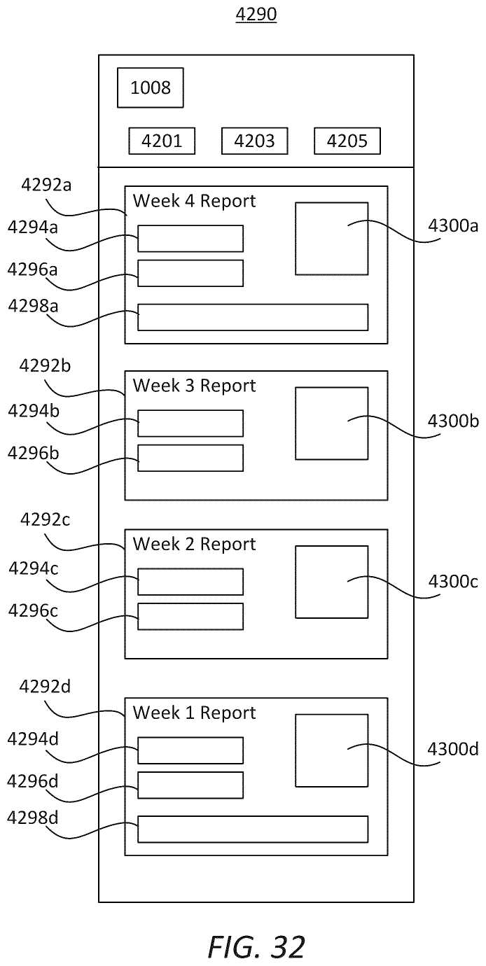

The glucose wellness application may prepare and display a weekly report or briefing. A notification or an alert may be sent to inform the user that the weekly report is available for viewing. Alternatively, the user may select the reports tab 4203, and GUI 4290 may be displayed that summarizes the user's weekly reports. s seen in FIG. 32, GUI 4290 may display a selectable card 4292a-4292d for each week that the user has used the glucose wellness application. The weeks may be displayed from earliest to latest or alternatively, latest to earliest. Each selectable card 4292a-4292d may list the dates 4294a-4294d for that particular week, the stage 4296a-4296d for that particular week, and a display 4300a-4300d of the average daily count for that week relative to the benchmark or target count for the week. In some embodiments, the display 4300a-4300d may be displayed as a fraction with the average daily count for that week in the numerator and the benchmark or target count for the week in the denominator. In some embodiments, the selectable card 4292a-4292d may include an additional message 4298a, 4298d that states that a stage has been completed.

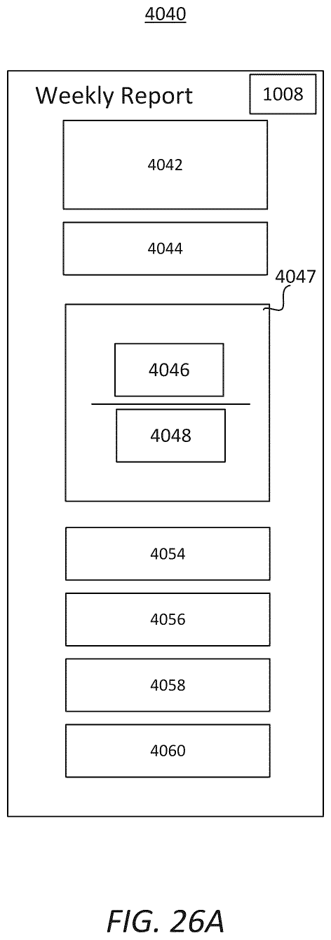

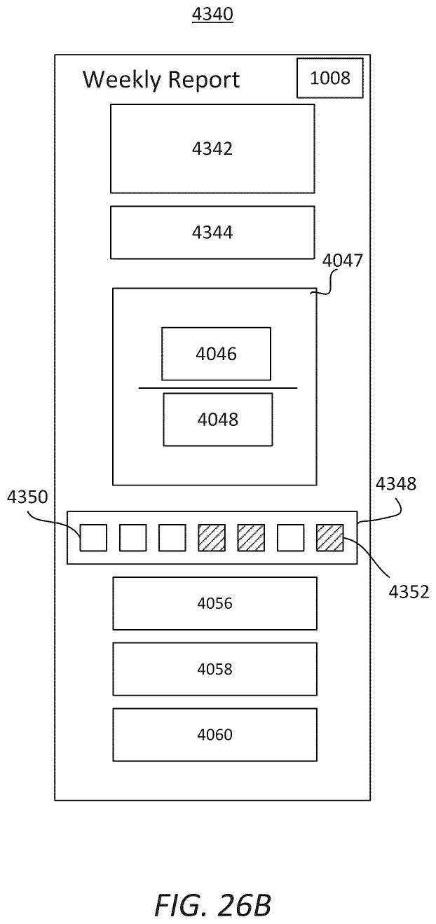

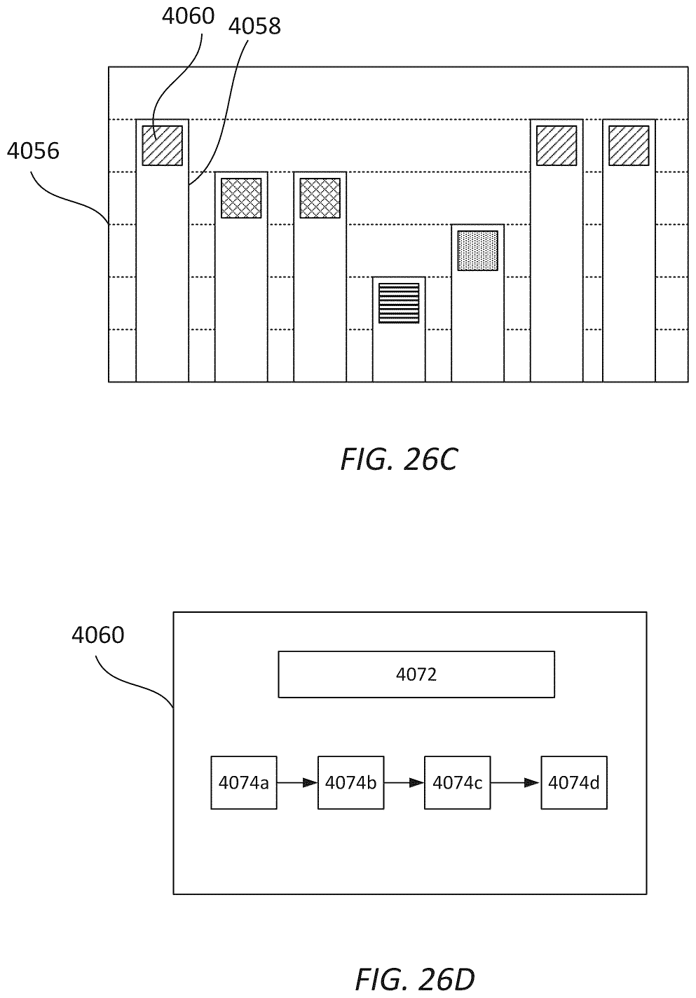

In some embodiments, as seen in FIG. 26A, the weekly report may be displayed in a GUI 4040 that includes a graphical display 4047 of the previous week's count spent on each day. The graphical display 4047 may have a portion corresponding to each time of day monitored. For example, the graphical display 4047 may have four portions corresponding to four times-of-day periods. These portions may or may not be clearly delineated. The average count total for each of the time-of-day periods may be displayed in each corresponding portion. The average count total 4046 earned for each day of the previous week may also be displayed. The average count total 4046 earned for each day may be displayed relative to the daily total budget 4026. The average count total 4046 earned for each day may be displayed as a numerator of a fraction and the daily total budget 4048 may be displayed as a denominator of the fraction.

In some embodiments, the graphical display 4047 may be a pie chart. In some embodiments, the graphical display 4047 may be a pie chart in the shape of an annular ring. In some embodiments, the graphical display 4047 may be a bar graph.

In some embodiments, a portion of the graph corresponding to the portion or times of the day with the highest count total may be highlighted in a different color than the rest of the portions of the graph.

In some embodiments, the fraction may be displayed in a center of the graphical display 4047.

In some embodiments, the graphical display 4047 may also highlight the time-of-day period that is the focus area.

The weekly report may include a message 4042 regarding the user's assigned glucose signature or profile based on the previous week's data. The message 4042 may identify the time-of-day period on which the user should focus.

The weekly report may also include a summary 4044 of the total count values for each of the days of the previous week(s). In some embodiments, the summary may include a graphic for each day that highlights the time-of-day period in which the highest number of counts were spent in a different color. In some embodiments, a graphic for a day in which the most counts were consumed may be highlighted. In other embodiments, the summary 4044 may display an entry for each day, which includes the counts for the day. The days in which the counts stayed within the benchmark or target goal may be highlighted.

The weekly report may also include an analysis 4054 of the past week's data. If events were logged, the analysis section 4054 may include a list of the food items or activities for which the most counts were spent.

The weekly report may also include a section on how the user's past week compares to others 4056. In some embodiments, the comparison 4056 may be to others in the user's age group. For example, the user's counts spent in the previous week may be compared to the counts spent by other users in the same age group. Alternatively, the user's target goal may be compared to other users' target goals in the same age group. The comparison section 4056 may include a graph of a count spread may highlight the user's counts and an average count value for users in the same age group. The age groups may be determined by decade.

The weekly report may also include a section that highlights the user's focus area 4058, or the time-of-day period on which the user should focus, and optionally provides additional details. The focus area section 4058 may also include tips and recommendations on how to lower the user's counts in this highlighted time-of-day period. In some embodiments, a user may be able to up vote or down vote (approve or disapprove, like or dislike) various tips, articles, and recommendations. Such analytics may then be used by the one or more processors of the wellness application to choose which content to present to the user based on their up votes and down votes.

The weekly report may also contain a new weekly counts budget section 4060. If the user was successful in staying within budget the past week, then the one or more processors of the wellness application may determine a new, lower counts budget for the next week. In some embodiments, the last week's counts budget and the new week's counts budget may be displayed side by side. The counts budgets may optionally be displayed graphically. For instance, last week's count budget may be displayed in a geometric shape having a first color and the new week's count budget may be displayed in a geometric shape having a second color.

In other embodiments, as seen in FIG. 26B, may be displayed in a GUI 4340 that may include a message 4342 regarding the user's progress the last week. The message 4342 may inform the user how their counts accrued the previous week compare to others in their age group. The message 4342 may also include the user's new target count.

The weekly report may include a statement 4344 that identifies the user's average count accrued in the previous week in text, and may also identify the time of day period in which most of the counts were accrued.

GUI 4340 may include a graphical display 4047 of the previous week's count spent on each day. The graphical display 4047 may have a portion corresponding to each time of day monitored. For example, the graphical display 4047 may have four portions corresponding to four times-of-day periods. These portions may or may not be clearly delineated. The average count total for each of the time-of-day periods may be displayed in each corresponding portion. The average count total 4046 earned for each day of the previous week may also be displayed. The average count total 4046 earned for each day may be displayed relative to the daily total budget 4026. The average count total 4046 earned for each day may be displayed as a numerator of a fraction and the daily total budget 4048 may be displayed as a denominator of the fraction.

In some embodiments, the graphical display 4047 may be a pie chart. In some embodiments, the graphical display 4047 may be a pie chart in the shape of an annular ring. In some embodiments, the graphical display 4047 may be a bar graph.

In some embodiments, a portion of the graph corresponding to the portion or times of the day with the highest count total may be highlighted in a different color than the rest of the portions of the graph.

In some embodiments, the fraction may be displayed in a center of the graphical display 4047.

In some embodiments, the graphical display 4047 may also highlight the time-of-day period that is the focus area.

The weekly report may also include a summary 4044 of the total count values for each of the days of the previous week(s). In some embodiments, the summary may include a graphic for each day that highlights the time-of-day period in which the highest number of counts were spent in a different color. In some embodiments, a graphic for a day in which the most counts were consumed may be highlighted. In other embodiments, the summary 4044 may display an entry for each day, which includes the counts for the day. The days in which the counts stayed within the benchmark or target goal may be highlighted.

The weekly report may also include a summary 4348 of the total count values for each of the days of the previous week(s). In some embodiments, the summary may include a graphic for each day that highlights the time-of-day period in which the highest number of counts were spent in a different color. In some embodiments, a graphic for a day in which the most counts were consumed may be highlighted. In other embodiments, as seen in FIG. 26B, the summary 4348 may display an entry for each day, which includes the numerical value of the counts for the day, along with the date and name of the day (Monday, Tuesday, etc.). The days in which the counts stayed within the benchmark or target goal may be highlighted (see, e.g., 4350). The days in which the user went above the benchmark or target goal may be shaded or otherwise distinguished (see, e.g., 4352).

The weekly report may also include a graph 4056 of the user's check-in replies regarding their chosen benefit (see FIG. 25C, 4039). As seen in FIG. 26C, the graph 4056 may be a bar graph that maps the users' responses with the days along the X-axis and the response level on the Y-axis. Each bar 4058 may contain an emoji or emoticon 4060 that corresponds to the entered response level.

The weekly report may also include a graph 4058 that illustrates the user's count relative to a relevant population. In some embodiments, the relevant population may be an age range. The graph may show the distribution of counts for the population and include a marker where the user's count falls on the graph. The graph may also contain a shaded portion that indicates the target range of counts.

The weekly report may also include a display 4060 of the user's new target count. The display 4060 may also include previous week's target counts, showing the progression to the current week. An exemplary display 4060 can be seen in FIG. 26D, which shows target or benchmark counts from previous weeks 4074a-4074c and the current target count 4074d, which is highlighted with a different color, shading, or other distinguishable feature. Display 4060 may also include a statement 4072 this tells the user what the new target count is.

### The User's Plan

In some embodiments, the glucose wellness application may divide the user's journey or plan into different phases. In embodiments where the glucose wellness application has different phases or stages, the information presented in the reports may vary depending on the phase or stage. The home or live screens may include an icon 1008 indicating the status of the biosensor, a progress tab 4201, a reports tab 4203, and an overview tab 4205. The home or live screens may also include quick links to today (home screen, live screen) 3862, the user's plan or journey 3864, the log 3867, a library of articles or informative descriptions (“discover”) 3868, and reports or progress 3866.

### First Stage

When the user first starts using the glucose wellness application, the user may be in the first phase or stage, e.g., a baseline stage. During the baseline stage, the primary focus is minimizing the counts that the user accrues, with the aim of the user staying within the benchmark goal set after the user enters their profile information. The benchmark goal may be based on the counts determined for a percentage of people in the user's age range. For example, the goal may be for the user to limit their counts each day to below the 95th percentile, alternatively the 90th percentile, alternatively the 85th percentile, alternatively the 80th percentile, alternatively the 75th percentile, for users in the user's age range. In some embodiments, users may have the option to adjust their count goal to make the daily goal either higher (easier to achieve) or lower (harder to achieve).

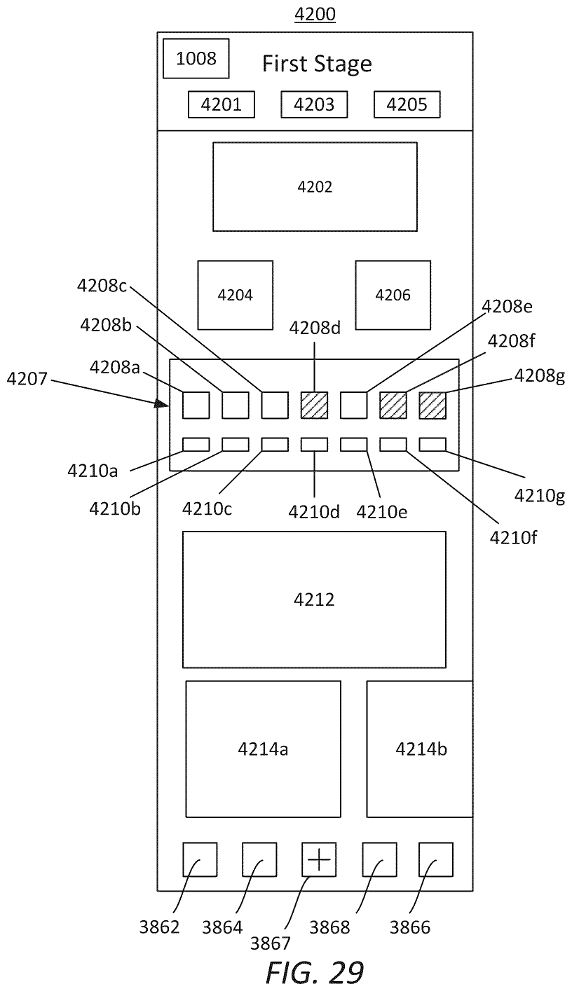

During the first stage, the user may access a stage one progress GUI 4200 by selecting progress tab 4201. As seen in FIG. 29, stage one progress GUI 4200 may include a link to a video or text 4202 that includes information regarding stage one, e.g., how best to utilize the application in phase one, what the user can expect, etc. Stage one progress GUI 4200 may also include a display of the benchmark target counts 4204 that was determined for the user. In some embodiments, the benchmark target counts 4204 may be displayed as a numerical value. GUI 4200 may also display the benefit or focus area 4206 that was selected by the user. The benefit or focus area 4206 may be displayed as text and/or a representative icon.

GUI 4200 may also display a summary 4207 of the user's counts for the current week. The summary 4207 may include a graphic 4208a-4208g for each day of the current week, along with a label for the day of the week and the date 4210a-4210g. The graphic 4208a-4208g may include the count value for the particular day. If the user stayed within the benchmark goal, then the graphic for that day may include a distinguishing feature, such as a bold or colored outline, or a different fill color (scc, e.g., 4208a-4208c, 4208c). Alternatively, or in addition to, days in which the user did not stay within the benchmark count may be shaded a different color (see, e.g., 4208d and 4208f-4208g). Alternatively, or in addition to, days in the future or days in which no data has been collected may be shaded yet a different color.

GUI 4200 may also include an explanatory text description 4212 of the first stage. During the first week, the user may see how their glucose reacts to their lifestyle in real time, practice the fundamental teachings of the glucose wellness application, and start experiencing holistic health benefits. The text description 4212 may also explain that the user can start making changes and finish the first week to move to the second stage or phase. GUI 4200 may also include articles and other informative content displayed in selectable cards 4214a-4214b. These selectable cards 4214a-4214b may present descriptions of various fundamental teachings of the glucose wellness application, including prioritizing protein, choosing vegetables first, eating your food in the right order to reduce your glucose spikes, etc.

### Second Stage

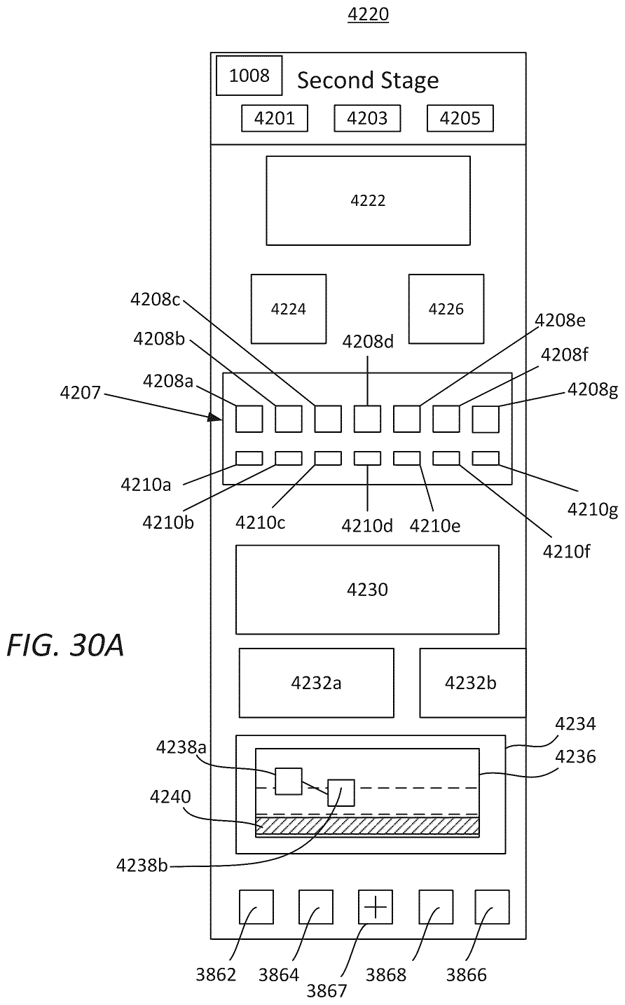

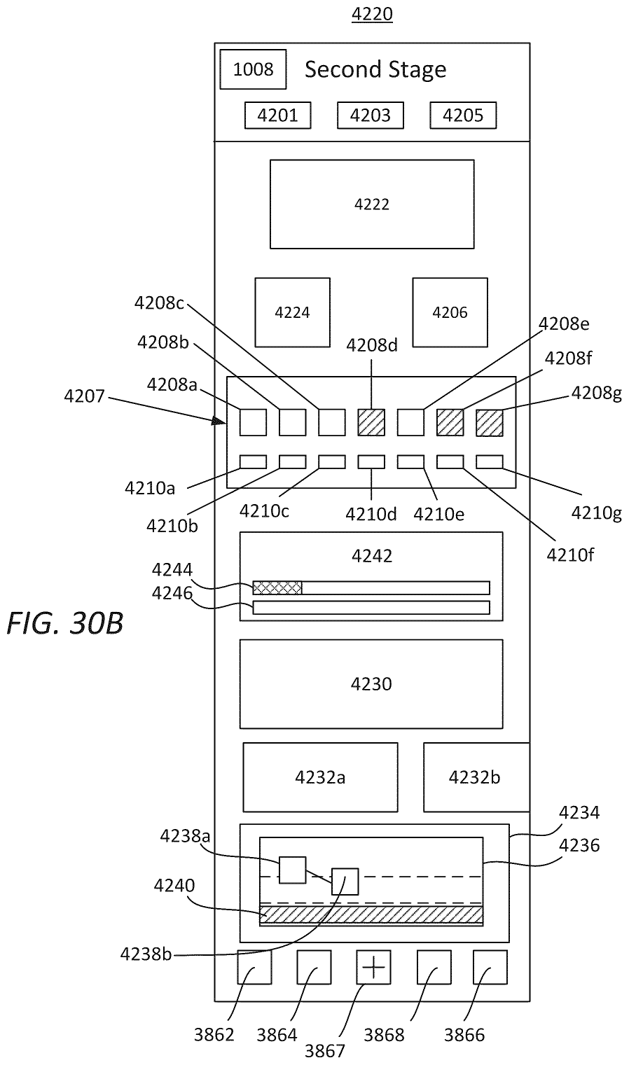

After the user completes the first stage, which in some cases may be the first week of usage, the user may enter the second stage. When the user selects the plan or journey link 3864 and the progress tab 4201, a different GUI 4220 may appear. As seen in FIGS. 30A-30B, the stage two GUI 4220 may include a link to a video or text 4222 that includes information regarding stage two, e.g., how the user may have a personalized target on which to focus based on the previous week's data. The user's goal in the second stage may be to stay within their target, lower their glucose spikes, and start building healthier habits. For example, the goal may be for the user to limit their counts each day to below a daily count for a median day achieved by the user in the first stage. The second stage may last multiple weeks in some embodiments. In such a case, in subsequent weeks, the goal may be for the user to limit their count each day to a certain percentage less than the median day of their best week achieved so far (e.g., their lowest median day). The certain percentage may be about 10% or less, alternatively 7% or less, alternatively 5% or less, alternatively 3% or less. In some embodiments, users may have the option to adjust their count goal to make the daily goal either higher (easier to achieve) or lower (harder to achieve).

Stage two progress GUI 4220 may also include a display of the target counts 4224 that was determined for the user. In some embodiments, the user's target counts 4224 may be displayed as a numerical value. GUI 4220 may also display the focus area 4226 that was determined for the user by one or more processors according to instructions stored in the memory of a computing device. The focus area 4226 may be displayed as text and/or a representative icon and may be a particular time of day period in which the user should try to reduce their glucose spikes. The focus areas may be, but are not limited to, morning, afternoon, evening, or overnight.

GUI 4220 may also display a summary 4207 of the user's counts for the current week. The summary 4207 may include a graphic 4208a-4208g for each day of the current week, along with a label for the day of the week and the date 4210a-4210g. The graphic 4208a-4208g may include the count value for the particular day. If the user stayed within the benchmark goal, then the graphic for that day may include a distinguishing feature, such as a bold or colored outline, or a different fill color (see, e.g., 4208a-4208c, 4208c). Alternatively, or in addition to, days in which the user did not stay within the benchmark count may be shaded a different color (see, e.g., 4208d and 4208f-4208g). Alternatively, or in addition to, days in the future or days in which no data has been collected may be shaded yet a different color.

GUI 4220 may also include an explanatory text description 4230 of what to expect in the second stage. During the second week, the user may get a new personalized target and focus area based on the previous week's data. The user's goal in the second stage is to stay within their target, lower their glucose spikes, and start building healthier habits. GUI 4220 may also include articles and other informative content displayed in selectable cards 4232a-4232b. These selectable cards 4232a-4232b may present recommendations on how to improve the user's focus area. For instance, one selectable card may state that protein and fat keep the user fuller longer and suggest a hard-boiled egg and avocado with their toast. Another card may suggest a smoothie and provide a recipe. The selectable cards 4232a-4232b may contain the description and/or also provide a link to a longer explanation or associated article.

GUI 4220 may also include a display of the user's progress over time 4234. The display 4234 may include a graph 4236 that graphs the user's average daily counts from the previous weeks. In some embodiments, each data count may represent an average daily count for a single week. For example, 4238a may be the average daily count for week one (stage one) and 4328b may be the average daily count for week two (stage two). The graph 4236 may also contain a target counts range 4240 that shows the goal range of counts for the user to obtain. In other embodiments, the graph may display the user's daily counts for a period of time, e.g., the current week.

The second stage may last multiple weeks. In some embodiments, as seen in FIG. 30B, the GUI 4220 may include a countdown section 4242 that illustrates the user's progress in stage two and the remaining time to get to stage three. The countdown display 4242 may include an explanation of the requirements for the user to complete stage two. In some embodiments, a user may be required to stay in their target count for a minimum number of days of the week for a minimum number of weeks in order to complete stage two. For instance, in some embodiments, the user may be required to stay within their target for at least 5 days a week for two weeks in order to complete stage two. The countdown display 4242 may include two progress indicators 4244, 4246. Progress indicator 4244 may illustrate how many days that the user's count has stayed under their target count that week. Progress indicator 4246 may indicate how many weeks the user has satisfied the requirement for staying under their target count for the required minimum number of days.

### Third Stage

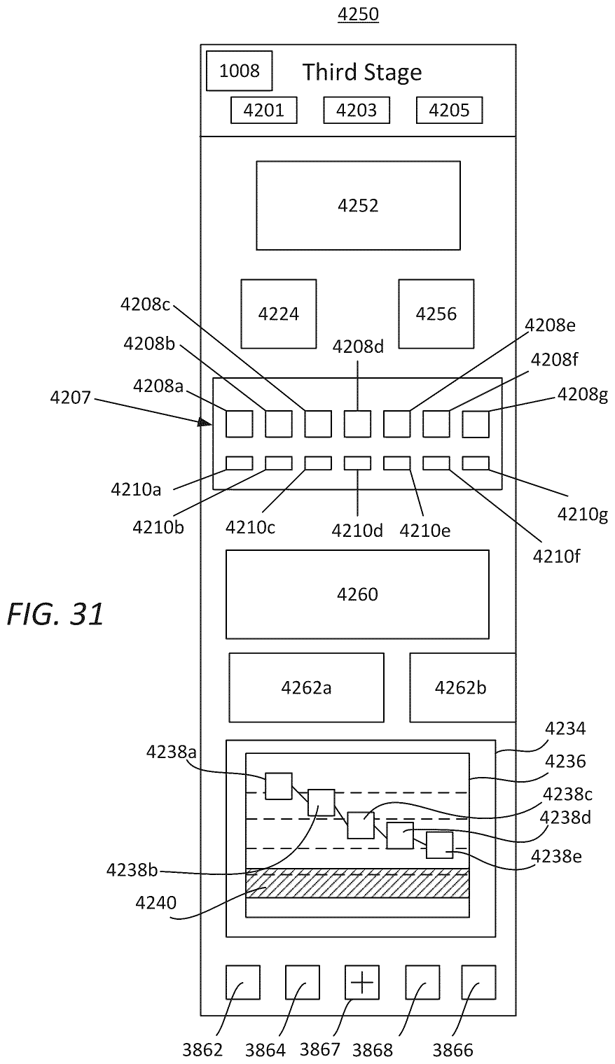

After the user satisfies the requirements to complete the second stage, the user may enter the third stage, e.g., the evolve stage. When the user selects the plan or journey link 3864 and the progress tab 4201, a different GUI 4250 may appear. As seen in FIG. 31, the stage three GUI 4250 may include a link to a video or text 4252 that includes information regarding stage three, e.g., how the user may work on their focus areas.

Stage three progress GUI 4250 may also include a display of the target counts 4224 that was determined for the user for that week of stage three. In some embodiments, the user's target counts 4224 may be displayed as a numerical value. GUI 4250 may also display the benefit 4206 that was selected by the user. The benefit 4206 may be displayed as text and/or a representative icon.

GUI 4250 may also display a summary 4207 of the user's counts for the current week. The summary 4207 may include a graphic 4208a-4208g for each day of the current week, along with a label for the day of the week and the date 4210a-4210g. The graphic 4208a-4208g may include the count value for the particular day. If the user stayed within the benchmark goal, then the graphic for that day may include a distinguishing feature, such as a bold or colored outline, or a different fill color (scc, e.g., 4208a-4208c, 4208e). Alternatively, or in addition to, days in which the user did not stay within the benchmark count may be shaded a different color (see, e.g., 4208d and 4208f-4208g). Alternatively, or in addition to, days in the future or days in which no data has been collected may be shaded yet a different color.

GUI 4250 may also include an explanatory text description 4260 of what to expect in the third stage. During the third stage, the user may move to working on their focus areas. The third stage may be the time for the user to move beyond making small changes and start building towards life-long habits. GUI 4250 may also include articles and other informative content displayed in selectable cards 4232a-4232b. These selectable cards 4232a-4232b may present recommendations on how to improve the user's focus area or benefit. For instance, one selectable card may state that protein and fat keep the user fuller longer and suggest a hard-boiled egg and avocado with their toast. Another card may suggest a smoothie and provide a recipe. The selectable cards 4262a-4262b may contain the description and/or also provide a link to a longer explanation or associated article.

GUI 4250 may also include a display of the user's progress over time 4234. The display 4234 may include a graph 4236 that graphs the user's average daily counts from the previous weeks. In some embodiments, each data count may represent an average daily count for a single week. For example, 4238a may be the average daily count for week one, 4328b may be the average daily count for week two, 4238c may be the average daily count for week three, 4238d may be the average daily count for week four, and 4238e may be the average daily count for week five. The graph 4236 may also contain a target counts range 4240 that shows the goal range of counts for the user to obtain. In other embodiments, the graph may display the user's daily counts for a period of time, e.g., the current week.

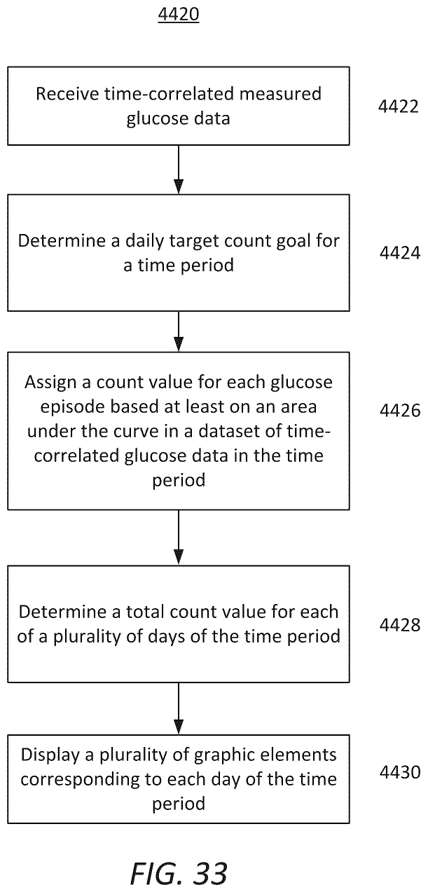

An exemplary method 4420 for displaying metrics relating to glucose management of a user is displayed in FIG. 33.

At Step 4422, time-correlated measured glucose data is received. According to some embodiments, the data can be received by a reader 120, local computer system 170, trusted computer system 180, or any computing device used in conjunction with an analyte monitoring system.

At Step 4424, a daily target count goal for a time period is determined. In some embodiments, the daily target count goal is determined based on a comparison to a distribution of the counts determined for a predetermined population. In some embodiments, the predetermined population is determined according to an age of the user. In some embodiments, the daily target count goal is determined based on a total count value determined for at least one day of a previous time period.

At Step 4426, a count value for each glucose episode is assigned based at least on an area under the curve of the each glucose episode in a dataset of time-correlated glucose data in the time period.

At Step 4428, a total count value for each of a plurality of days of the time period is determined.

At Step 4430, a plurality of graphic elements corresponding to each day of the time period. In some embodiments, each of the plurality of graphic elements comprises the total count value determined for each of the plurality of days of the time period.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible. The embodiments described herein are restated and expanded upon in the following paragraphs without explicit reference to the figures.

In many embodiments, a system for monitoring metrics relating to a user includes: wireless communications circuitry configured to receive measured glucose data; a display configured to visually present information; and one or more processors coupled with the wireless communications circuitry, the display, and a memory storing instructions that, when executed by the one or more processors, cause the system to: identify, in a dataset of time correlated measured glucose data, a first alert point for a potential glucose episode if the last received glucose data point satisfies at least one alert condition; identify a first potential local minimum in a first time period, wherein the first time period comprises a beginning data point and the first alert point; confirm the first potential local minimum as a first start point of a first glucose episode if the first potential local minimum satisfies at least one local minimum condition; calculate an integrated area under a curve over time of a first portion of the graph of the dataset beginning at the first start point of the first glucose episode to the first alert point; and assign a first count value for the first portion.

In some embodiments, the at least one alert condition comprises confirming that a calculated rate of change between the first alert point and a previous point within about 20 minutes of the first alert point is above an alert rate of change threshold.

In some embodiments, the at least one alert condition comprises confirming that a difference between the first alert point and the first potential local minimum is above a local minimum alert threshold.

In some embodiments, the at least one alert condition comprises confirming that a calculated integrated area under the curve from the first potential local minimum to the alert point corresponds to a count value above a threshold count value.

In some embodiments, the dataset comprises a graph of glucose levels vs. time.

In some embodiments, the integrated area under the curve over time of the first portion is calculated with respect to a non-zero base value. In some embodiments, the non-zero base value is between about 60 mg/dL to about 100 mg/dL. In some embodiments, the non-zero base value is about 70 mg/dL.

In some embodiments, the beginning data point is a determined end point of a previous adjacent glucose episode.

In some embodiments, the beginning data point has a timestamp that is between about 60 to about 90 minutes before a timestamp of the last received glucose data point.

In some embodiments, the first count value is assigned based on a comparison to a distribution of a glucose metric determined from a predetermined population. In some embodiments, the glucose metric is an integrated area under a curve over time.

In some embodiments, the first potential local minimum is confirmed as the first start point of the first glucose episode if a rate of change between the first potential local minimum and a previous point within the first time period is above a rate of change threshold value. In some embodiments, the previous point is within about 20 minutes of the first potential local minimum. In some embodiments, the previous point is a previous closest local minimum.

In some embodiments, the instructions, when executed by the one or more processors, further cause the system to display the first count value.

In some embodiments, the instructions, when executed by the one or more processors, further cause the system to: identify, in the dataset, at least one additional potential local minimum in the first time period; and confirm the at least one additional potential local minimum as the first start point if the at least one additional potential local minimum satisfies the at least one local minimum condition. In some embodiments, the step of identifying the at least one additional potential local minimum in the first time period is performed after the first potential local minimum is confirmed as a start point if the first potential local minimum satisfies the at least one local minimum condition. In some embodiments, the at least one additional potential local minimum is a single additional potential local minimum.

In some embodiments, the instructions, when executed by the one or more processors, further cause the system to output a notification that an episode is occurring after the first alert point is identified and the first start point is confirmed. In some embodiments, the notification comprises the first count value for the first portion.

In some embodiments, the instructions, when executed by the one or more processors, further cause the system to: confirm that a last received glucose data point in a second time period is part of the first glucose episode; calculate an integrated area under the curve over time of a second portion of the graph beginning at the first start point to the last received glucose data point in the second time period; and assign a second count value for the second portion.

In some embodiments, the instructions, when executed by the one or more processors, further cause the system to display the second count value.

In some embodiments, the integrated area under the curve over time of the second portion is calculated with respect to a non-zero base value.

In some embodiments, the instructions, when executed by the one or more processors, further cause the system to: identify a first potential end point of the first glucose episode; calculate an integrated area under the curve over time of a portion of the graph from the first start point to the first potential end point; assign a total count value for the integrated area under the curve over time of the portion of the graph from the first start point to the first potential end point; and confirm the first potential end point as a first end point of the first glucose episode if the first potential end point satisfies at least one end point condition.

In some embodiments, the at least one end point is confirmed if the total count value is less than a threshold count value. In some embodiments, the first potential end point is confirmed as the first end point if a difference between a glucose level of the first start point of the first glucose episode and a glucose level of the first potential end point is below a threshold difference. In some embodiments, the first potential end point is confirmed as the first end point if the first potential end point is a local minimum as compared to a previous adjacent data point. In some embodiments, the first potential end point is confirmed as the first end point if a calculated integrated area under the curve over time of a portion of the graph from the start point to the first potential end point is less than a minimum episode point threshold score.

In some embodiments, the instructions, when executed by the one or more processors, further cause the system to: identify, in the dataset, at least one potential local minimum in at least one additional time period, wherein the at least one additional time period comprises a beginning data point and a last received glucose data point in the at least one additional time period; confirm at least one potential local minimum as a start point of at least one additional glucose episode if the at least one potential local minimum satisfies the at least one local minimum condition; calculate an integrated area under the curve over time of at least one additional portion of the graph from the start point of the at least one additional glucose episode to the last received glucose data point in the at least one additional time period; and assign at least one additional first count value for the at least one additional portion.

In some embodiments, the instructions, when executed by the one or more processors, further cause the system to: identify at least one additional potential end point of the at least one additional glucose episode; confirm the at least one additional potential end point as an end point of the at least one additional glucose episode if the at least one additional potential end point satisfies the at least one end point condition; calculate an integrated area under the curve over time of the at least one additional portion of the graph from the start point of at least one additional glucose episode to the at least one additional potential end point; and assign a total count value for the integrated area under the curve over time of the at least one additional portion of the graph from the start of at least one additional glucose episode to the at least one additional potential end point.

In some embodiments, the at least one end point is confirmed if the total count value for the integrated area under the curve over time of the at least one additional portion of the graph is less than threshold count value.

In some embodiments, the wireless communications circuitry is further configured to receive input related to exercise, and wherein the instructions, when executed by the one or more processors, further cause the system to: determine that the first glucose episode is associated with exercise; and disregard the calculated integrated area under the curve over time for the first glucose episode.

In some embodiments, the wireless communications circuitry is further configured to receive input related to exercise from a user, and the instructions, when executed by the one or more processors, further cause the system to: determine if the first glucose episode is flagged by a user to not assign a count value for the first glucose episode; and disregard the calculated integrated area under the curve over time for the first glucose episode if the first glucose episode was flagged by the user.

In many embodiments, a system for determining metrics relating to a user includes: wireless communications circuitry configured to receive time-correlated measured glucose data; a display configured to visually present information; and one or more processors coupled with the wireless communications circuitry, the display, and a memory storing instructions that, when executed by the one or more processors, cause the system to: determine a glucose metric for each of a plurality of glucose episodes in a dataset of time-correlated glucose data in a time period; assign a count value for each glucose episode of the plurality of glucose episodes based on a comparison of the determined glucose metric to a distribution of glucose metrics determined from a predetermined population; determine an aggregate count value for each of a plurality of time-of-day periods; and assign a glycemic profile from a plurality of glycemic profiles based on the determined aggregate count value for each of the plurality of time-of-day periods.

In some embodiments, the glucose metric comprises a calculated integrated areas under a curve over time of a graph of the dataset for each glucose episode of the plurality of glucose episodes.

In some embodiments, the plurality of time-of-day periods comprises at least 3 time-of-day periods.

In some embodiments, the plurality of time-of-day periods comprises a morning period, an afternoon period, an evening period, and an overnight period.

In some embodiments, the aggregate count value for each of the plurality of time-of-day periods is determined by averaging a count total for each time-of-day period in the time period.

In some embodiments, the aggregate count value for each of the plurality of time-of-day periods is determined by identifying a median count total for each time-of-day period in the time period.

In some embodiments, the aggregate count value for each of the plurality of time-of-day periods is determined by determining a sum of a count total for each time-of-day period in the time period.

In some embodiments, the time period is about 1 week.

In some embodiments, the glycemic profile is assigned based on a determined time-of-day period having a highest aggregate count value. In some embodiments, a first glycemic profile is assigned if the determined aggregate count value is highest in a morning time-of-day period. In some embodiments, a second glycemic profile is assigned if the determined aggregate count value is highest in an afternoon time-of-day period. In some embodiments, a third glycemic profile is assigned if the determined aggregate count value is highest in an evening time-of-day period. In some embodiments, a fourth glycemic profile is assigned if the determined aggregate count value is highest in an overnight time-of-day period. In some embodiments, wherein a fifth glycemic profile is assigned if the determined aggregate count values for at least two time-of-day periods are equal.

In some embodiments, the instructions, when executed by the one or more processors, further cause the system to output a recommendation based on the assigned glycemic profile. In some embodiments, the recommendation is further based on at least one characteristic of a user selected from the group consisting of an age, a height, a weight, a BMI, a gender, a race, and an ethnicity. In some embodiments, the recommendation is further based on at least one input logged by a user, the at least one input selected from the group consisting of food, stress, sleep, mood, and exercise. In some embodiments, the recommendation is further based on a particular geographic location of a user.

In some embodiments, the count value is assigned based on a population distribution of integrated areas under the curve linearized to a range of count values.

In many embodiments, a system for monitoring metrics relating to a user includes: wireless communications circuitry configured to receive time-correlated measured glucose data; a display configured to visually present information; and one or more processors coupled with the wireless communications circuitry, the display, and a memory storing instructions that, when executed by the one or more processors, cause the system to: assign a count value for each glucose episode based at least on an area under a curve of the each glucose episode in a dataset of time-correlated glucose data; calculate a running sum of count values for a plurality of glucose episodes in a time period; and display a progress indicator representative of the running sum of the count values relative to a target count goal for the time period.

In some embodiments, the count value is assigned based on a comparison to a distribution of a glucose metric determined from a predetermined population.

In some embodiments, the dataset comprises a graph of glucose data vs. time.

In some embodiments, the time period is about one day.

In some embodiments, the progress indicator comprises a display of a fraction comprising the running sum of count values in a numerator and the target count goal in a denominator.

In some embodiments, the display of the fraction has a first end, a second end, and a length, and wherein a numerical value of the running sum of count values is displayed at a position along the length of the numerator that is proportional to [the running sum of count values]/[the target count goal] if the running sum of count values is less than or equal to the target count goal.

In some embodiments, the target count goal is displayed in the denominator near the second end.

In some embodiments, the display of the fraction has a first end, a second end, and a length, and wherein a numerical value of the target count goal is displayed at a position along the length of the denominator that is proportional to [the target count goal]/[the running sum of count values] if the running sum of count values is greater than the target count goal. In some embodiments, wherein a numerical value of the running sum of count values is displayed at the first end.

In some embodiments, when the numerical value of the running sum of count values is zero, the numerical value of the running sum of count values is displayed near the first end.

In some embodiments, the instructions, when executed by the one or more processors, further cause the system to: determine a difference between a first count value assigned in a first time period and a second count value assigned in a second time period, wherein the second time period is immediately after the first time period; assign a count trend status for the second time period from a plurality of count trend statuses based on the determined difference; and display a color representative of the assigned count trend status for the second time period.

In some embodiments, the plurality of count trend statuses comprises a balanced status, a declining in spike status, a flat during spike status, and a rising in spike status.

In some embodiments, the first time period and the second time period both occur during a single glucose episode.

In some embodiments, the progress indicator is displayed in a graphic user interface (GUI), and wherein the color is displayed as a background color of the GUI.

In some embodiments, wherein the display comprises a graphic user interface, and wherein the color representative of the assigned count trend status is displayed as a background color of a graphic user interface (GUI).

In some embodiments, the instructions, when executed by the one or more processors, further cause the system to: determine a difference between at least one additional count value in at least one additional time period and the second count value, wherein the at least one additional time period is immediately after the second time period; assign a count trend status for the at least one additional time period from a plurality of count trend statuses based on the determined difference; and display a color representative of the assigned count trend status for the at least one additional time period.

In some embodiments, the color representative of the assigned count trend status for the at least one additional time period is displayed as a background color of a graphic user interface (GUI).

In some embodiments, the color representative of the assigned count trend status for the at least one additional time period is displayed as a background color of a first portion of the GUI, and the color representative of the assigned count trend status for the second time period is displayed in a second portion of the GUI. In some embodiments, the first portion of the GUI is a top portion and wherein the second portion of the GUI is a bottom portion. In some embodiments, the color representative of the assigned count trend status for the at least one additional time period and the color representative of the assigned count trend status for the second time period are displayed as blended colors. In some embodiments, the color representative of the assigned count trend status for the at least one additional time period and the color representative of the assigned count trend status for the second time period are displayed in a gradient.

In some embodiments, the instructions, when executed by the one or more processors, further cause the system to: determine a slope for a line formed by count values determined for a plurality of periods of time; assign a count trend status for at least one of the plurality of periods of time based on the determined slope; and display a color representative of the assigned count trend status for the second time period.

In some embodiments, the plurality of count trend statuses comprises a balanced status, a declining in spike status, a flat during spike status, and a rising in spike status. In some embodiments, the count trend status is rising in spike if the slope is positive. In some embodiments, the count trend status is declining in spike if the slope is negative. In some embodiments, the count trend status is flat in spike if the slope is substantially constant.

In some embodiments, the plurality of periods of time are consecutive.

In some embodiments, a system for monitoring metrics relating to a user includes: wireless communications circuitry configured to receive time-correlated measured glucose data; a display configured to visually present information; and one or more processors coupled with the wireless communications circuitry, the display, and a memory storing instructions that, when executed by the one or more processors, cause the system to: assign a count for each glucose episode based at least on an area under a curve of the each glucose episode in a dataset of time-correlated glucose data; determine a difference between a first count value assigned to a first glucose episode in a first time period and a second count value assigned to a second glucose episode in a second time period, wherein the second time period is immediately after the first time period; and assign a count trend status for the second time period based on the determined difference between the first count value and the second count value, wherein the count trend status is one of a plurality of count trend statuses.

In some embodiments, the instructions, when executed by the one or more processors, further cause the system to display a color representative of the assigned count trend status for the second time period.

In some embodiments, the plurality of count trend statuses comprises a balanced status, a declining in spike status, a flat during spike status, and a rising in spike status.

In some embodiments, the first time period and the second time period both occur during a single glucose episode.

In many embodiments, a system for monitoring metrics relating to a user includes: wireless communications circuitry configured to receive time-correlated measured glucose data; a display configured to visually present information; and one or more processors coupled with the wireless communications circuitry, the display, and a memory storing instructions that, when executed by the one or more processors, cause the system to: assign a first count value for a glucose episode in a first time period and a second count value for a glucose episode in a second time period, wherein each of the first and second count values are based at least on an area under a curve of the each glucose episode in a dataset of time-correlated glucose data; determine a slope for a line formed by the first count value and the second count value; and assign a count trend status for one of the first or second time periods based on the determined slope.

In some embodiments, the instructions, when executed by the one or more processors, further cause the system to display a color representative of the assigned count trend status for the second time period.

In some embodiments, the count trend status is rising in spike if the slope is positive.

In some embodiments, the count trend status is declining in spike if the slope is negative.

In some embodiments, the count trend status is flat in spike if the slope is substantially constant.

In some embodiments, the glucose episode in the first time period and the glucose episode in the second time period are parts of a single glucose episode.

In some embodiments, the glucose episode in the first time period and the glucose episode in the second time period are different glucose episodes.

In many embodiments, a system for monitoring metrics relating to a user includes: wireless communications circuitry configured to receive time-correlated measured glucose data; a display configured to visually present information; and one or more processors coupled with the wireless communications circuitry, the display, and a memory storing instructions that, when executed by the one or more processors, cause the system to: assign a count value for each glucose episode based at least on an area under a curve of the each glucose episode in a dataset of time-correlated glucose data; display a summary graphic comprising a total count value for each of a plurality of time-of-day periods for a day, wherein the total count value for each of the plurality of time-of-day periods is a sum of the count values for each plurality of glucose episodes occurring during each of the plurality of time-of-day periods; and display a total count value for the day relative to a target count goal for the day.

In some embodiments, the summary graphic comprises four portions corresponding to four time-of-day periods, each portion comprising a numerical display of the total count value for one of the four time-of-day periods. In some embodiments, a portion corresponding to a highest total count value is a different color than a rest of the four portions. In some embodiments, the summary graphic is a pie chart. In some embodiments, the summary graphic is bar graph. In some embodiments, the summary graphic is circular graphic. In some embodiments, the display of the total count value for the day relative to the target count goal for the day is located in a center of the summary graphic.

In some embodiments, the instructions, when executed by the one or more processors, further cause the system to display text identifying a time-of-day period having a highest total count value.

In some embodiments, the instructions, when executed by the one or more processors, further cause the system to display a recommendation based on the total count value for the day.

In some embodiments, the instructions, when executed by the one or more processors, further cause the system to display a recommendation based on the determined time-of-day period having a highest total count value.

In some embodiments, the instructions, when executed by the one or more processors, further cause the system to display a list of untagged events from the day.

In some embodiments, the instructions, when executed by the one or more processors, further cause the system to display a prompt for a user to tag events detected during the day.

In some embodiments, the instructions, when executed by the one or more processors, further cause the system to display recommendations related to prioritizing protein.

In many embodiments, a system for monitoring metrics relating to a user includes: wireless communications circuitry configured to receive time-correlated measured glucose data; a display configured to visually present information; and one or more processors coupled with the wireless communications circuitry, the display, and a memory storing instructions that, when executed by the one or more processors, cause the system to: assign a count value for each glucose episode in a dataset of time-correlated glucose data based at least on an area under a curve of the each glucose episode; determine an aggregate total daily count value during a time period; determine an aggregate count value for each of a plurality of time-of-day periods during the time period; and display a summary graphic comprising the aggregate count value for each of the plurality of time-of-day periods, the aggregate total daily count value during the time period, and a target count goal for the day.

In some embodiments, the count value is assigned based on a comparison to a distribution of a glucose metric determined from a predetermined population. In some embodiments, the glucose metric determined from the predetermined population is an area under a curve.

In some embodiments, the time period is one week.

In some embodiments, the aggregate total daily count value is an average total daily count value, wherein the aggregate count value for each of a plurality of time-of-day periods is an average count value for each of a plurality of time-of-day periods.

In some embodiments, the summary graphic is a circular graphic, and wherein the aggregate total daily count value during the time period and the target count goal for the day are displayed in a center of the circular graphic.

In some embodiments, each of the aggregate count values for each of a plurality of time-of-day periods during the time period are displayed in a different portion of the circular graphic.

In some embodiments, a portion of the circular graphic corresponding to a time-of-day period with a highest aggregate count value is a different color than a rest of the circular graphic. In some embodiments, the time-of-day periods are arranged clockwise in the circular graphic.

In some embodiments, the summary graphic is a bar graph, and wherein bars corresponding to days having an aggregate total daily count higher than the target count goal comprise a first color and bars corresponding to days having an aggregate total daily count lower than or equal to the target count goal comprise a second color.

In some embodiments, the instructions, when executed by the one or more processors, further cause the system to display a summary for each day of the time period.

In some embodiments, the summary for each day comprises a daily count total and a graphic highlighting a time-of-day period with a highest count value.

In some embodiments, the instructions, when executed by the one or more processors, further cause the system to display a comparison of the determined aggregate total daily count value to a plurality of total daily count values for a population. In some embodiments, the population is related to an age of a user.

In some embodiments, the instructions, when executed by the one or more processors, further cause the system to display a recommended time-of-day period for a user to mitigate.

In some embodiments, the instructions, when executed by the one or more processors, further cause the system to: determine a new target count goal based on a sum of the assigned count value for each glucose episode in the time period; and display the new target count goal.

In some embodiments, the instructions, when executed by the one or more processors, further cause the system to: assign a glycemic profile from a plurality of glycemic profiles based on the determined aggregate count value for each of the plurality of time of day periods; and display the assigned glycemic profile.

In some embodiments, the time period is one week, and the instructions, when executed by the one or more processors, further cause the system to display a plurality of graphic elements corresponding to each day of the time period, wherein each of the plurality of graphic elements comprises the aggregate total daily count value determined for each day of the time period. In some embodiments, a graphic element of the plurality of graphic elements having an aggregate total daily count value equal to or below the target count goal for the day is visually distinguishable from a graphic element of the plurality of graphic elements having an aggregate total daily count value above the target count goal for the day.

In some embodiments, the instructions, when executed by the one or more processors, further cause the system to display a graph comprising the determined aggregate total daily count value during the time period and at least one aggregate daily count value determined from an earlier time period.

In some embodiments, the instructions, when executed by the one or more processors, further cause the system to display a focus area identified by the user.

In some embodiments, the wireless communications circuitry is configured to receive data related to a focus area identified by the user, and wherein the instructions, when executed by the one or more processors, further cause the system to display a graph of answers from a user prompt related to the focus area identified by the user.

In some embodiments, the graph is a bar graph.

In some embodiments, the instructions, when executed by the one or more processors, further cause the system to display a graphic comprising the target count goal for the day for the time period and at least one target count goal for the day for a previous time period.

In many embodiments, a system for monitoring metrics relating to a user includes: wireless communications circuitry configured to receive measured glucose data; a display configured to visually present information; and one or more processors coupled with the wireless communications circuitry, the display, and a memory storing instructions that, when executed by the one or more processors, cause the system to: identify, in a dataset of time correlated glucose data, a first potential local minimum in a first time period, wherein the first time period comprises a beginning data point and a last received glucose data point in the first time period; confirm the first potential local minimum as a first start point of a first glucose episode if the first potential local minimum satisfies at least one local minimum condition; calculate an integrated area under a curve over time of a first portion of a graph of the dataset beginning at the first start point of the first glucose episode to the last received glucose data point; and assign a first count value for the first portion.

In some embodiments, the beginning data point is a determined end point of a previous adjacent glucose episode.

In some embodiments, the beginning data point has a timestamp that is between about 60 to about 90 minutes before a timestamp of the last received glucose data point.

In some embodiments, the first count value is assigned based on a comparison to a distribution of a glucose metric determined from a predetermined population. In some embodiments, the glucose metric determined from the predetermined population is an integrated area under a curve over time.

In some embodiments, the dataset is a graph of glucose vs. time.

In some embodiments, the first potential local minimum is confirmed as the first start point of the first glucose episode if a rate of change between the first potential local minimum and a previous point within the first time period is above a rate of change threshold value. In some embodiments, the previous point is within about 20 minutes of the first potential local minimum. In some embodiments, the previous point is a previous closest local minimum.

In some embodiments, the instructions, when executed by the one or more processors, further cause the system to display the first count value.

In some embodiments, the instructions, when executed by the one or more processors, further cause the system to: identify, in the dataset, at least one additional potential local minimum in the first time period; and confirm the at least one additional potential local minimum as the first start point if the at least one additional potential local minimum satisfies the at least one local minimum condition.

In some embodiments, the step of identifying the at least one additional potential local minimum in the first time period is performed after the first potential local minimum is confirmed as a start point if the first potential local minimum satisfies the at least one local minimum condition.

In some embodiments, the instructions, when executed by the one or more processors, further cause the system to identify, in the dataset, a first alert point for a potential glucose episode if the last received glucose data point in the first time period satisfies at least one alert condition.

In some embodiments, the at least one alert condition comprises confirming that a calculated rate of change between the first alert point and a previous point within about 20 minutes of the first alert point is above an alert rate of change threshold.

In some embodiments, the at least one alert condition comprises confirming that a difference between the first alert point and the first potential local minimum is above a local minimum alert threshold.

In some embodiments, the at least one alert condition comprises confirming that a calculated integrated area under the curve from the first potential local minimum to the alert point corresponds to a count value above a threshold count value.

In some embodiments, the at least one local minimum condition comprises determining if a difference between a glucose level of the first alert point and a glucose level of the first potential local minimum is above a threshold difference.

In some embodiments, the instructions, when executed by the one or more processors, further cause the system to: confirm that a last received glucose data point in a second time period is part of the first glucose episode; calculate an integrated area under the curve over time of a second portion of the graph beginning at the first start point to the last received glucose data point in the second time period; and assign a second count value for the second portion.

In some embodiments, the instructions, when executed by the one or more processors, further cause the system to display the second count value.

In some embodiments, the instructions, when executed by the one or more processors, further cause the system to: identify a first potential end point of the first glucose episode; calculate an integrated area under the curve over time of a portion of the graph from the first start point to the first potential end point; assign a total count value for the integrated area under the curve over time of the portion of the graph from the first start point to the first potential end point; and confirm the first potential end point as a first end point of the first glucose episode if the first potential end point satisfies at least one end point condition.

In some embodiments, the at least one end point is confirmed if the total count value is less than a threshold count value.

In some embodiments, the first potential end point is confirmed as the first end point if a difference between a glucose level of the first start point of the first glucose episode and a glucose level of the first potential end point is below a threshold difference.

In some embodiments, the first potential end point is confirmed as the first end point if the first potential end point is a local minimum as compared to a previous adjacent data point.

In some embodiments, the first potential end point is confirmed as the first end point if a calculated integrated area under the curve over time of a portion of the graph from the start point to the first potential end point is less than a minimum episode point threshold score.

In some embodiments, the instructions, when executed by the one or more processors, further cause the system to: identify, in the graph of glucose data vs. time, at least one potential local minimum in at least one additional time period, wherein the at least one additional time period comprises a beginning data point and a last received glucose data point in the at least one additional time period; confirm at least one potential local minimum as a start point of at least one additional glucose episode if the at least one potential local minimum satisfies the at least one local minimum condition; calculate an integrated area under the curve over time of at least one additional portion of the graph from the start point of the at least one additional glucose episode to the last received glucose data point in the at least one additional time period; and assign at least one additional first count value for the at least one additional portion.

In some embodiments, the instructions, when executed by the one or more processors, further cause the system to: identify at least one additional potential end point of the at least one additional glucose episode; confirm the at least one additional potential end point as an end point of the at least one additional glucose episode if the at least one additional potential end point satisfies the at least one end point condition; calculate an integrated area under the curve over time of the at least one additional portion of the graph from the start point of at least one additional glucose episode to the at least one additional potential end point; and assign a total count value for the integrated area under the curve over time of the at least one additional portion of the graph from the start of at least one additional glucose episode to the at least one additional potential end point.

In some embodiments, the at least one end point is confirmed if the total count value for the integrated area under the curve over time of the at least one additional portion of the graph is less than threshold count value.

In some embodiments, the wireless communications circuitry is further configured to receive input related to exercise, and the instructions, when executed by the one or more processors, further cause the system to: determine that the first glucose episode is associated with exercise; and disregard the calculated integrated area under the curve over time for the first glucose episode.

In some embodiments, the wireless communications circuitry is further configured to receive input related to exercise from a user, and the instructions, when executed by the one or more processors, further cause the system to: determine if the first glucose episode is flagged by a user; and disregard the calculated integrated area under the curve over time for the first glucose episode if the first glucose episode was flagged by a user.

In many embodiments, a system for determining metrics relating to a user includes: wireless communications circuitry configured to receive measured glucose data; a display configured to visually present information; and one or more processors coupled with the wireless communications circuitry, the display, and a memory storing instructions that, when executed by the one or more processors, cause the system to: assign a count value for each glucose episode of a plurality of glucose episodes in a dataset of time-correlated glucose data based at least on an area under the curve of the each glucose episode; categorize each of the plurality of glucose episodes as a food event or a non-food event; and calculate a first running sum of count values for each glucose episode categorized as a food event.

In some embodiments, the instructions, when executed by the one or more processors, further cause the system to calculate a second running sum of count values for each glucose episode categorized as a non-food event.

In some embodiments, the instructions, when executed by the one or more processors, further cause the system to output the second running sum of count values on the display.

In some embodiments, the instructions, when executed by the one or more processors, further cause the system to output the first running sum of count values on the display.

In some embodiments, the count value for each glucose episode is assigned based on a calculated integrated area under the curve over time for each glucose episode.

In many embodiments, a system for determining metrics relating to a user includes: wireless communications circuitry configured to receive measured glucose data; a display configured to visually present information; and one or more processors coupled with the wireless communications circuitry, the display, and a memory storing instructions that, when executed by the one or more processors, cause the system to: determine an area under a curve for each glucose episode of a plurality of glucose episodes in a dataset of time-correlated glucose data; assign a count value for each glucose episode of the plurality of glucose episodes based on a comparison of the determined area under the curve to a distribution of areas under the curve determined from a predetermined population; determine an aggregate daily total count value for a first time period based on the assigned count value for each glucose episode of the plurality of glucose episodes; and determine a target daily count goal for a user for a second time period based on the determined aggregate daily total count value for the first time period.

In some embodiments, the target daily count goal for the user for the second time period is determined based on a comparison of the determined aggregate daily total count value for the first time period to a population of users having an aggregate daily total count value within a threshold of the determined aggregate daily total count value of the user.

In some embodiments, the first time period comprises a first week.

In some embodiments, the second time period comprises a second week, wherein the second week occurs after the first week.

In some embodiments, the first week and second week are consecutive.

In many embodiments, a system for monitoring metrics relating to a user includes: wireless communications circuitry configured to receive measured glucose data; a display configured to visually present information; and one or more processors coupled with the wireless communications circuitry, the display, and a memory storing instructions that, when executed by the one or more processors, cause the system to: identify, in a dataset of time-correlated glucose data, a plurality of local maxima as potential peaks and a plurality of local minima as potential troughs in a time period; screen the potential peaks and the potential troughs to determine a plurality of glucose episodes that satisfy at least one condition; calculate an integrated area under a curve of the dataset over time for each of the plurality of glucose episodes; and assign a count value to each of the plurality of glucose episodes.

In some embodiments, the plurality of local maxima and the plurality of local minima are screened by applying an algorithm.

In some embodiments, the dataset of time-correlated glucose data comprises a graph of glucose data vs. time.

In some embodiments, the algorithm detects an episode in the plurality of glucose episodes if a rate of change between an identified peak and an identified trough is greater than a threshold rate of change.

In some embodiments, the algorithm detects an episode in the plurality of glucose episodes if a time difference between an identified peak and an identified trough is greater than a threshold time difference.

In many embodiments, a system for monitoring metrics relating to a user includes: wireless communications circuitry configured to receive time-correlated measured glucose data; a display configured to visually present information; and one or more processors coupled with the wireless communications circuitry, the display, and a memory storing instructions that, when executed by the one or more processors, cause the system to: determine a start point of a glucose episode in a dataset of time-correlated glucose data; assign a first count value for a first portion of a glucose episode based at least on an area under a curve of the dataset over time, wherein the first portion of the glucose episode begins at the start point and extends to a last received glucose data point in a first time period; assign a second count value for a second portion of the glucose episode based at least on an area under the curve, wherein the second portion of the glucose episode begins at the last received glucose data point in the first time period and extends to a last received glucose data point in a second time period, wherein the second time period is immediately after the first time period; determine a difference between the first count value and the second count value; assign a count trend status for the second time period from a plurality of count trend statuses based on the determined difference; and display a graph of glucose data vs. time, wherein a portion of the graph corresponding to the second time period is displayed in a color representative of the assigned count trend status.

In some embodiments, the dataset comprises a graph of glucose data vs. time.

In some embodiments, the instructions, when executed by the one or more processors, further cause the system to: assign at least one additional count value for at least one additional portion of the glucose episode based at least on an area under the curve, wherein the at least one additional portion of the graph beginning at the last received glucose data point in the at least one additional time period to the last received glucose data point in the at least one additional time period, wherein the at least one additional time period is immediately after the second time period; determine a difference between the at least one additional count value and the second count value; assign a count trend status for the at least one additional time period from a plurality of count trend statuses based on the determined difference; and display the graph of glucose data vs. time, wherein a portion of the graph corresponding to the at least one additional time period is displayed in a color representative of the assigned count trend status for the at least one additional time period.

In some embodiments, at least a portion of an area under the curve of the graph of glucose data vs. time for the second time period comprises the color representative of the assigned count trend status for the second time period.

In some embodiments, the first count value and the second count value are each assigned based on a comparison to a distribution of areas under the curve determined from a predetermined population.

In some embodiments, the instructions, when executed by the one or more processors, further cause the system to: calculate a running count value for the glucose episode; and display the running count value near the glucose episode in the graph of glucose data vs. time.

In some embodiments, the instructions, when executed by the one or more processors, further cause the system to: calculate a total count value for the glucose episode; and display the total count value near the glucose episode in the graph of glucose data vs. time.

In some embodiments, a y-axis of the graph of glucose data vs. time represents glucose levels.

In some embodiments, the y-axis is not labeled with numerical values.

In some embodiments, the instructions, when executed by the one or more processors, further cause the system to display a numerical value of a glucose level in the graph of glucose data vs. time in response to a user scrolling through the graph.

In some embodiments, the instructions, when executed by the one or more processors, further cause the system to display a numerical value of a glucose level in the graph of glucose data vs. time in response to a user applying pressure to a point on the graph.

In some embodiments, the wireless communications circuitry is further configured to receive time-correlated logged data, and the instructions, when executed by the one or more processors, further cause the system to display an icon related to logged data on the graph of glucose data vs. time near a time associated with the logged data. In some embodiments, the logged data comprises lifestyle events, activity events, food, or combinations thereof.

In some embodiments, the time-correlated measured glucose data is received about every 5 minutes.

In some embodiments, the time-correlated measured glucose data is received about every 15 minutes.

In many embodiments, a system for monitoring metrics relating to glucose management of a user includes: wireless communications circuitry configured to receive time-correlated measured glucose data and data related answers from at least one prompt to a user; a display configured to visually present information; and one or more processors coupled with the wireless communications circuitry, the display, and a memory storing instructions that, when executed by the one or more processors, cause the system to: determine a daily target count goal for a time period; assign a count value for each glucose episode based at least on an area under a curve of the each glucose episode in a dataset of time-correlated glucose data in the time period; determine a total count value for each of a plurality of days of the time period; and display a plurality of graphic elements corresponding to each day of the time period, wherein each of the plurality of graphic elements comprises the total count value determined for each of the plurality of days of the time period.

In some embodiments, the count value is assigned based on a comparison to a distribution of areas under the curve determined from a predetermined population.

In some embodiments, the time period is one week.

In some embodiments, a graphic element of the plurality of graphic elements corresponding to a first day of the time period comprises a total daily count value determined for the first day.

In some embodiments, a graphic element of the plurality of graphic elements having a total daily count value equal to or below the daily target count goal is visually distinguishable from a graphic element of the plurality of graphic elements having a total daily count value above the daily target count goal.

In some embodiments, the instructions, when executed by the one or more processors, further cause the system to display an indication of a focus area for the user.

In some embodiments, the focus area is selected from the group consisting of boost energy, manage hunger, improve mood, better sleep, and stay focused.

In some embodiments, the daily target count goal is determined based on a comparison to a distribution of the counts determined for a predetermined population.

In some embodiments, the predetermined population is determined according to an age of the user.

In some embodiments, the daily target count goal is determined based on a total count value determined for at least one day of a previous time period.

In some embodiments, the instructions, when executed by the one or more processors, further cause the system to display a recommended time-of-day period for a user to mitigate.

In some embodiments, the instructions, when executed by the one or more processors, further cause the system to: determine an aggregate total daily count value for the time period and an aggregate total daily count value for at least one previous time period; and display a graph comprising the aggregate total daily count value for the time period and the aggregate total daily count value for the at least one previous time period.

In some embodiments, the aggregate total daily count value for the time period comprises the average total daily count value for the time period, and wherein the aggregate total daily count value for the at least one previous time period comprises the average total daily count value for the at least one previous time period.

In some embodiments, the glucose management comprises at least a first stage and a second stage, wherein advancement of the user from the first stage to the second stage requires the user to satisfy at least one requirement, wherein the instructions, when executed by the one or more processors, further cause the system to display at least one progress indicator relating to the satisfaction of the at least one requirement.

In some embodiments, the at least one requirement comprises the user having a total daily count value equal to or below the daily target count goal for a minimum number of days in the time period.

In some embodiments, the minimum number of days is about 5 days and the time period is about one week.

In some embodiments, the at least one requirement further comprises the user having a total daily count value equal to or below the daily target count goal for about 5 days in a one week time period for at least two weeks.

All references mentioned in the specification and the appendix are hereby expressly incorporated by reference in their entireties for all purposes.

### CONCLUSION

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Thus, the foregoing description of specific embodiments of the disclosed subject matter has been presented for purposes of illustration and description. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents. Furthermore, any features, functions, steps, or elements of the embodiments may be recited in or added to the claims, as well as negative limitations that define the inventive scope of the claims by features, functions, steps, or elements that are not within that scope.

As used herein, the term ‘curve’ may relate to a graph of time-correlated data over time, which may be a graph of measured glucose data, and/or may relate to a dataset of time-correlated data which may comprise a graph of glucose data vs. time. As used herein, the term ‘glucose metric’ may refer to a glucose variability metric, an area-under-the-curve over time, or an integrated area-under-the-curve over time, and/or a calculated area under a curve for one or more glucose episodes. For example, the glucose metric may be an integrated area under the curve over time of a first portion of a graph of a dataset of time-correlated measured glucose data, optionally beginning at a first start point of the first glucose episode to a first alert point.

In many embodiments, a system for determining metrics relating to a subject includes: an input configured to receive measured glucose data; a display configured to visually present information; and one or more processors coupled with the input, the display, and a memory storing instructions that, when executed by the one or more processors, cause the system to: determine a plurality of glucose metrics for each of a plurality of glucose spikes in a graph of glucose data vs. time over a time period; assign a count value for each glucose metric of the plurality of glucose metrics based on a comparison to a distribution of the glucose metric determined from a predetermined population; determine an aggregate count value for each of a plurality of time-of-day periods during the time period; and assign a glycemic profile from a plurality of glycemic profiles based on the determined aggregate count value for each of the plurality of time-of-day periods.

In some embodiments, the plurality of glucose metrics comprises a plurality of calculated integrated areas under the curve for each of the plurality of glucose spikes.

In some embodiments, the plurality of time-of-day periods comprises at least 3 time of day periods.

In some embodiments, the plurality of time-of-day periods comprises a morning period, an afternoon period, an evening period, and an overnight period.

In some embodiments, the aggregate count value for each of the plurality of time-of-day periods is determined by averaging a count total for each time-of-day period in the time period.

In some embodiments, the aggregate count value for each of the plurality of time-of-day periods is determined by identifying a median count total for each time-of-day period in the time period.

In some embodiments, the aggregate count value for each of the plurality of time-of-day periods is determined by determining a sum of a count total for each time-of-day period in the time period.

In some embodiments, the time period is about 1 week.

In some embodiments, the glycemic profile is assigned based on a determined time-of-day period having a highest aggregate count value. In some embodiments, a first glycemic profile is assigned if the determined aggregate count value is highest in a morning time-of-day period. In some embodiments, a second glycemic profile is assigned if the determined aggregate count value is highest in an afternoon time-of-day period. In some embodiments, a third glycemic profile is assigned if the determined aggregate count value is highest in an evening time-of-day period. In some embodiments, a fourth glycemic profile is assigned if the determined aggregate count value is highest in an overnight time-of-day period. In some embodiments, a fifth glycemic profile is assigned if the determined aggregate count values for at least two time-of-day periods are equal.

In some embodiments, the instructions, when executed by the one or more processors, further cause the system to output a recommendation based on the assigned glycemic profile. In some embodiments, the recommendation is further based on at least one characteristic of a user selected from the group consisting of an age, a height, a weight, a BMI, a gender, a race, and an ethnicity. In some embodiments, the recommendation is further based on at least one input logged by a user, the at least one input selected from the group consisting of food, stress, sleep, mood, and exercise. In some embodiments, the recommendation is further based on a particular geographic location of a user.

In some embodiments, the count value is assigned based on a population distribution of integrated areas under the curve linearized to a range of count values.

In many embodiments, a system for monitoring and/or displaying metrics relating to a subject includes: an input configured to receive time-correlated measured glucose data; a display configured to visually present information; and one or more processors coupled with the input, the display, and a memory storing instructions that, when executed by the one or more processors, cause the system to: determine a plurality of glucose metrics for at least one glucose spike in a dataset of time-correlated glucose data; assign a count value for each glucose metric of the plurality of glucose metrics; calculate a running sum of the count values for each glucose metric assigned in a time period; and display a progress indicator representative of the running sum of the count values relative to a total count goal for the time period.

In some embodiments, the count value is assigned based on a comparison to a distribution of the glucose metric determined from a predetermined population.

In some embodiments, the dataset comprises a graph of glucose data vs. time.

In some embodiments, the time period is about one day.

In some embodiments, the plurality of glucose metrics are determined for a single glucose spike.

In some embodiments, the plurality of glucose metrics are determined for a plurality of glucose spikes.

In some embodiments, the progress indicator comprises a display of a fraction comprising the running sum of count values in a numerator and the total count goal in a denominator. In some embodiments, the display of the fraction has a first end, a second end, and a length, and wherein a numerical value of the running sum of count values is displayed at a position along the length of the numerator that is proportional to [the running sum of count values]/[the total count goal] if the running sum of count values is less than or equal to the total count goal. In some embodiments, the total count goal is displayed in the denominator near the second end. In some embodiments, the display of the fraction has a first end, a second end, and a length, and wherein a numerical value of the total count goal is displayed at a position along the length of the denominator that is proportional to [the total count goal]/[the running sum of count values] if the running sum of count values is greater than the total count goal. In some embodiments, a numerical value of the running sum of count values is displayed at the first end. In some embodiments, when the numerical value of the running sum of count values is zero, the numerical value of the running sum of count values is displayed near the first end.

In some embodiments, the instructions, when executed by the one or more processors, further cause the system to: determine a difference between a first count value assigned in a first time period and a second count value assigned in a second time period, wherein the second time period is immediately after the first time period; assign a count trend status for the second time period from a plurality of count trend statuses based on the determined difference; and display a color representative of the assigned count trend status for the second time period.

In some embodiments, the plurality of count trend statuses comprises a balanced status, a declining in spike status, a flat during spike status, and a rising in spike status.

In some embodiments, the first time period and the second time period both occur during a single glucose spike.

In some embodiments, the progress indicator is displayed in a graphic user interface (GUI), and wherein the color is displayed as a background color of the GUI.

In some embodiments, the color representative of the assigned count trend status is displayed as a background color of a graphic user interface (GUI).

In some embodiments, the instructions, when executed by the one or more processors, further cause the system to: determine a difference between the at least one additional count value and the second count value; assign a count trend status for the at least one additional time period from a plurality of count trend statuses based on the determined difference; and display a color representative of the assigned count trend status for the at least one additional time period. In some embodiments, the color representative of the assigned count trend status for the at least one additional time period is displayed as a background color of a graphic user interface (GUI).

In some embodiments, the color representative of the assigned count trend status for the at least one additional time period is displayed as a background color of a first portion of the GUI, and the color representative of the assigned count trend status for the second time period is displayed in a second portion of the GUI. In some embodiments, the first portion of the GUI is a top portion and wherein the second portion of the GUI is a bottom portion. In some embodiments, the color representative of the assigned count trend status for the at least one additional time period and the color representative of the assigned count trend status for the second time period are displayed as blended colors. In some embodiments, the color representative of the assigned count trend status for the at least one additional time period and the color representative of the assigned count trend status for the second time period are displayed in a gradient.

In some embodiments, the instructions, when executed by the one or more processors, further cause the system to: determine a slope for a line formed by count values determined for a plurality of periods of time; assign a count trend status for at least one of the plurality of periods of time based on the determined slope; and display a color representative of the assigned count trend status for the second time period.

In some embodiments, the plurality of count trend statuses comprises a balanced status, a declining in spike status, a flat during spike status, and a rising in spike status.

In some embodiments, the count trend status is rising in spike if the slope is positive.

In some embodiments, the count trend status is declining in spike if the slope is negative.

In some embodiments, the count trend status is flat in spike if the slope is substantially constant.

In some embodiments, the plurality of periods of time are consecutive.

In many embodiments, a system for monitoring and/or displaying metrics relating to a subject includes: an input configured to receive time-correlated measured glucose data; a display configured to visually present information; and one or more processors coupled with the input, the display, and a memory storing instructions that, when executed by the one or more processors, cause the system to: determine a plurality of glucose metrics for at least one glucose spike in a graph of glucose data vs. time; assign a count value for each glucose metric of the plurality of glucose metrics; determine a difference between a first count value assigned in a first time period and a second count value assigned in a second time period, wherein the second time period is immediately after the first time period; and assign a count trend status for the second time period from a plurality of count trend statuses based on the determined difference.

In some embodiments, the instructions, when executed by the one or more processors, further cause the system to display a color representative of the assigned count trend status for the second time period.

In some embodiments, the plurality of count trend statuses comprises a balanced status, a declining in spike status, a flat during spike status, and a rising in spike status.

In some embodiments, the first time period and the second time period both occur during a single glucose spike.

In many embodiments, a system for monitoring and/or displaying metrics relating to a subject includes: an input configured to receive time-correlated measured glucose data; a display configured to visually present information; and one or more processors coupled with the input, the display, and a memory storing instructions that, when executed by the one or more processors, cause the system to: determine a plurality of glucose metrics for at least one glucose spike in a graph of glucose data vs. time; assign a count value for each glucose metric of the plurality of glucose metrics; determine a slope for a line formed by count values determined for a plurality of periods of time; and assign a count trend status for at least one of the plurality of periods of time based on the determined slope.

In some embodiments, the instructions, when executed by the one or more processors, further cause the system to: display a color representative of the assigned count trend status for the second time period.

In some embodiments, the count trend status is rising in spike if the slope is positive.

In some embodiments, the count trend status is declining in spike if the slope is negative.

In some embodiments, the count trend status is flat in spike if the slope is substantially constant.

In many embodiments, a system for monitoring and/or displaying metrics relating to a subject includes: an input configured to receive time-correlated measured glucose data; a display configured to visually present information; and one or more processors coupled with the input, the display, and a memory storing instructions that, when executed by the one or more processors, cause the system to: determine a plurality of glucose metrics for at least one glucose spike in a graph of glucose data vs. time; assign a count value for each glucose metric of the plurality of glucose metrics; display a summary graphic comprising a total count value for the glucose metric for each of a plurality of time-of-day periods for a day, wherein the total count value for each of the plurality of time-of-day periods is a sum of the count values for each glucose metric determined for a glucose spike occurring during each of the plurality of time-of-day periods; and display a total count value for the day relative to a total count goal for the day.

In some embodiments, the summary graphic comprises four portions corresponding to four time-of-day periods, each portion comprising a numerical display of the total count value for one of the four time-of-day periods. In some embodiments, a portion corresponding to a highest total count value is a different color than a rest of the four portions. In some embodiments, the summary graphic is a pie chart. In some embodiments, the summary graphic is bar graph. In some embodiments, the summary graphic is circular graphic. In some embodiments, the display of the total count value for the day relative to the total count goal for the day is located in a center of the summary graphic.

In some embodiments, the instructions, when executed by the one or more processors, further cause the system to display text identifying a time-of-day period having a highest total count value.

In some embodiments, the instructions, when executed by the one or more processors, further cause the system to display a recommendation based on the total count value for the day.

In some embodiments, the instructions, when executed by the one or more processors, further cause the system to display a recommendation based on the determined time-of-day period having a highest total count value.

In some embodiments, the instructions, when executed by the one or more processors, further cause the system to display a list of untagged events from the day.

In some embodiments, the instructions, when executed by the one or more processors, further cause the system to display a prompt for a user to tag events detected during the day.

In some embodiments, the instructions, when executed by the one or more processors, further cause the system to display recommendations related to prioritizing protein.

In many embodiments, a system for monitoring and/or displaying metrics relating to a subject includes: an input configured to receive time-correlated measured glucose data; a display configured to visually present information; and one or more processors coupled with the input, the display, and a memory storing instructions that, when executed by the one or more processors, cause the system to: determine a plurality of glucose metrics for each of a plurality of glucose spikes in a graph of glucose data vs. time in a time period; assign a count value for each glucose metric of the plurality of glucose metrics in the time period; determine an aggregate total daily count value during the time period; determine an aggregate count value for each of a plurality of time-of-day periods during the time period; and display a summary graphic comprising the aggregate count value for each of the plurality of time-of-day periods, the aggregate total daily count value during the time period, and a total count goal for the day.

In some embodiments, the count value is assigned based on a comparison to a distribution of the glucose metric determined from a predetermined population.

In some embodiments, the time period is one week.

In some embodiments, the aggregate total daily count value is an average total daily count value, wherein the aggregate count value for each of a plurality of time-of-day periods is an average count value for each of a plurality of time-of-day periods.

In some embodiments, the summary graphic is circular graphic, and wherein the total daily count value during the time period and the total count goal for the day are displayed in a center of the circular graphic. In some embodiments, each of the aggregate count values for each of a plurality of time-of-day periods during the time period are displayed in a different portion of the circular graphic. In some embodiments, a portion of the circular graphic corresponding to a time-of-day period with a highest aggregate count value is a different color than a rest of the circular graphic. In some embodiments, the time-of-day periods are arranged clockwise in the circular graphic.

In some embodiments, the summary graphic is a bar graph, and wherein bars corresponding to days having the aggregate total daily count higher than the total count goal comprise a first color and bars corresponding to days having the aggregate total daily count lower than or equal to the total count goal comprise a second color.

In some embodiments, the instructions, when executed by the one or more processors, further cause the system to display a summary for each day of the time period. In some embodiments, the summary for each day comprises a count total and a graphic highlighting a time-of-day period with a highest count value.

In some embodiments, wherein the instructions, when executed by the one or more processors, further cause the system to display a comparison of the determined plurality of glucose metrics to a plurality of glucose metrics for a population. In some embodiments, the population is related to an age of a user.

In some embodiments, the instructions, when executed by the one or more processors, further cause the system to display recommended time-of-day period for a user to mitigate.

In some embodiments, the instructions, when executed by the one or more processors, further cause the system to: determine a new total count goal based on the assigned count value for each glucose metric of the plurality of glucose metrics in the time period; and display the new total count goal.

In some embodiments, the instructions, when executed by the one or more processors, further cause the system to: assign a glycemic profile from a plurality of glycemic profiles based on the determined aggregate count value for each of the plurality of time of day periods; and display the assigned glycemic profile.

In some embodiments, the time period is one week, and the instructions, when executed by the one or more processors, further cause the system to: display a plurality of graphic elements corresponding to each day of the time period, wherein each of the plurality of graphic elements comprises the aggregate total daily count value determined for each day of the time period.

In some embodiments, a graphic element of the plurality of graphic elements having an aggregate total daily count value equal to or below the total count goal for the day is visually distinguishable from a graphic element of the plurality of graphic elements having an aggregate total daily count value above the total count goal for the day.

In some embodiments, the instructions, when executed by the one or more processors, further cause the system to: display a graph comprising the determined aggregate total daily count value during the time period and at least one aggregate daily count value determined from an earlier time period.

In some embodiments, the instructions, when executed by the one or more processors, further cause the system to: display a focus area identified by the user.

In some embodiments, the input is configured to receive data related to a focus area identified by the user, and the instructions, when executed by the one or more processors, further cause the system to: display a graph of answers from a user prompt related to the focus area identified by the user. In some embodiments, the graph is a bar graph.

In some embodiments, the instructions, when executed by the one or more processors, further cause the system to: display a graphic comprising the total count goal for the day for the time period and at least one total count goal for the day for a previous time period.

In many embodiments, a system for determining metrics relating to a subject includes: an input configured to receive measured glucose data; a display configured to visually present information; and one or more processors coupled with the input, the display, and a memory storing instructions that, when executed by the one or more processors, cause the system to: determine a plurality of glucose metrics for each of a plurality of glucose spikes in a graph of glucose data vs. time over a time period; determine if each of the plurality of glucose spikes relates to a food event or relates to a non-food event; assign a count value for each glucose metric of the plurality of glucose metrics based on a comparison to a distribution of the glucose metric determined from a predetermined population; and calculate a first running sum of count values for each glucose metric determined for each of the plurality of glucose spikes related to a food event.

In some embodiments, the instructions, when executed by the one or more processors, further cause the system to calculate a second running sum of count values for each glucose metric determined for each of the plurality of glucose spikes related to a non-food event.

In some embodiments, the instructions, when executed by the one or more processors, further cause the system to output the second running sum of count values on a display.

In some embodiments, the instructions, when executed by the one or more processors, further cause the system to output the first running sum of count values on a display.

In some embodiments, the plurality of glucose metrics comprises a plurality of calculated integrated areas under the curve over time for each of the plurality of glucose spikes.

In many embodiments, a system for determining metrics relating to a subject includes: an input configured to receive measured glucose data; a display configured to visually present information; and one or more processors coupled with the input, the display, and a memory storing instructions that, when executed by the one or more processors, cause the system to: determine a plurality of glucose metrics for each of a plurality of glucose spikes in a graph of glucose data vs. time in each day of a first time period; assign a count value for each glucose metric of the plurality of glucose metrics based on a comparison to a distribution of the glucose metric determined from a predetermined population; determine an aggregate daily total count value for the first time period based on the assigned count values for each glucose metric of the plurality of glucose metrics; and determine a target daily count goal for a user for a second time period based on the determined aggregate daily total count value for the first time period.

In some embodiments, the target daily count goal for the user for the second time period is determined based on a comparison of the determined aggregate daily total count value for the first time period to a population of users having an aggregate daily total count value within a threshold of the determined aggregate daily total count value of the user.

In some embodiments, the first time period comprises a first week. In some embodiments, the second time period comprises a second week, wherein the second week occurs after the first week. In some embodiments, the first week and second week are consecutive.

In many embodiments, a system for monitoring and/or displaying metrics relating to glucose management of a user includes: an input configured to receive time-correlated measured glucose data and data related answers from at least one prompt to a user; a display configured to visually present information; and one or more processors coupled with the input, the display, and a memory storing instructions that, when executed by the one or more processors, cause the system to: determine a plurality of glucose metrics for each of a plurality of glucose spikes in a graph of glucose data vs. time in a time period; assign a count value for each glucose metric of the plurality of glucose metrics in the time period; determine a total count value for at least one day of the time period; determine a daily target count goal for the time period; and display a plurality of graphic elements corresponding to each day of the time period, wherein each of the plurality of graphic elements comprises the total count value determined for the at least one day of the time period.

In some embodiments, the count value is assigned based on a comparison to a distribution of the glucose metric determined from a predetermined population.

In some embodiments, the time period is one week.

In some embodiments, a graphic element of the plurality of graphic elements corresponding to a first day of the time period comprises a total daily count value determined for the first day.

In some embodiments, a graphic element of the plurality of graphic elements having a total daily count value equal to or below the daily target count goal is visually distinguishable from a graphic element of the plurality of graphic elements having a total daily count value above the daily target count goal.

In some embodiments, the instructions, when executed by the one or more processors, further cause the system to: display an indication of a focus area for the user. In some embodiments, the focus area is selected from the group consisting of boost energy, manage hunger, improve mood, better sleep, and stay focused.

In some embodiments, the daily target count goal is determined based on a comparison to a distribution of the counts determined for a predetermined population. In some embodiments, the predetermined population is determined according to an age of the user.

In some embodiments, the daily target count goal is determined based on a total count value determined for at least one day of a previous time period.

In some embodiments, the instructions, when executed by the one or more processors, further cause the system to: display a recommended time-of-day period for a user to mitigate.

In some embodiments, the instructions, when executed by the one or more processors, further cause the system to: determine an aggregate total daily count value for the time period and an aggregate total daily count value for at least one previous time period; and display a graph comprising the aggregate total daily count value for the time period and the aggregate total daily count value for the at least one previous time period. In some embodiments, the aggregate total daily count value for the time period comprises the average total daily count value for the time period, and wherein the aggregate total daily count value for the at least one previous time period comprises the average total daily count value for the at least one previous time period.

In some embodiments, the glucose management comprises at least a first stage and a second stage, wherein advancement of the user from the first stage to the second stage requires the user to satisfy at least one requirement, wherein the instructions, when executed by the one or more processors, further cause the system to: display at least one progress indicator relating to the satisfaction of the at least one requirement.

In some embodiments, the at least one requirement comprises the user having a total daily count value equal to or below the daily target count goal for a minimum number of days in the time period. In some embodiments, the minimum number of days is about 5 days and the time period is about one week. In some embodiments, the at least one requirement further comprises the user having a total daily count value equal to or below the daily target count goal for about 5 days in a one week time period for at least two weeks.

### Clauses

Exemplary embodiments are set out in the following numbered clauses.

Clause 1. A system for monitoring metrics relating to a user, the system comprising:
wireless communications circuitry configured to receive measured glucose data;
a display configured to visually present information; and
one or more processors coupled with the wireless communications circuitry, the display, and a memory storing instructions that, when executed by the one or more processors, cause the system to:
identify, in a dataset of time correlated measured glucose data, a first alert point for a potential glucose episode if the last received glucose data point satisfies at least one alert condition;
   identify a first potential local minimum in a first time period, wherein the first time period comprises a beginning data point and the first alert point;
confirm the first potential local minimum as a first start point of a first glucose episode if the first potential local minimum satisfies at least one local minimum condition;
calculate an integrated area under a curve over time of a first portion of a graph of the dataset beginning at the first start point of the first glucose episode to the first alert point; and assign a first count value for the first portion.

Clause 2. The system of clause 1, wherein the at least one alert condition comprises confirming that a calculated rate of change between the first alert point and a previous point within about 20 minutes of the first alert point is above an alert rate of change threshold.

Clause 3. The system of any of clauses 1-2, wherein the at least one alert condition comprises confirming that a difference between the first alert point and the first potential local minimum is above a local minimum alert threshold.

Clause 4. The system of any of clauses 1-3, wherein the at least one alert condition comprises confirming that a calculated integrated area under the curve from the first potential local minimum to the alert point corresponds to a count value above a threshold count value.

Clause 5. The system of any of clauses 1-4, wherein the dataset comprises a graph of glucose levels vs. time.

Clause 6. The system of any of clauses 1-5, wherein the integrated area under the curve over time of the first portion is calculated with respect to a non-zero base value.

Clause 7. The system of clause 6, wherein the non-zero base value is between about 60 mg/dL to about 100 mg/dL.

Clause 8. The system of clause 6, wherein the non-zero base value is about 70 mg/dL.

Clause 9. The system of any of clauses 1-8, wherein the beginning data point is a determined end point of a previous adjacent glucose episode.

Clause 10. The system of any of clauses 1-9, wherein the beginning data point has a timestamp that is between about 60 to about 90 minutes before a timestamp of the last received glucose data point.

Clause 11. The system of any of clauses 1-10, wherein the first count value is assigned based on a comparison to a distribution of a glucose metric determined from a predetermined population.

Clause 12. The system of any of clauses 1-11, wherein the first potential local minimum is confirmed as the first start point of the first glucose episode if a rate of change between the first potential local minimum and a previous point within the first time period is above a rate of change threshold value.

Clause 13. The system of clause 12, wherein the previous point is within about 20 minutes of the first potential local minimum.

Clause 14. The system of clause 12, wherein the previous point is a previous closest local minimum.

Clause 15. The system of any of clauses 1-14, wherein the instructions, when executed by the one or more processors, further cause the system to:
display the first count value.

Clause 16. The system of any of clauses 1-15, wherein the instructions, when executed by the one or more processors, further cause the system to:
identify, in the dataset, at least one additional potential local minimum in the first time period; and
confirm the at least one additional potential local minimum as the first start point if the at least one additional potential local minimum satisfies the at least one local minimum condition.

Clause 17. The system of clause 16, wherein the step of identifying the at least one additional potential local minimum in the first time period is performed after the first potential local minimum is confirmed as a start point if the first potential local minimum satisfies the at least one local minimum condition.

Clause 18. The system of clause 16, wherein the at least one additional potential local minimum is a single additional potential local minimum.

Clause 19. The system of any of clauses 1-18, wherein the instructions, when executed by the one or more processors, further cause the system to:
output a notification that an episode is occurring after the first alert point is identified and the first start point is confirmed.

Clause 20. The system of clause 19, wherein the notification comprises the first count value for the first portion.

Clause 21. The system of any of clauses 1-20, wherein the instructions, when executed by the one or more processors, further cause the system to:
confirm that a last received glucose data point in a second time period is part of the first glucose episode;
calculate an integrated area under the curve over time of a second portion of the graph beginning at the first start point to the last received glucose data point in the second time period; and
assign a second count value for the second portion.

Clause 22. The system of clause 21, wherein the instructions, when executed by the one or more processors, further cause the system to:
display the second count value.

Clause 23. The system of clause 21, wherein the integrated area under the curve over time of the second portion is calculated with respect to a non-zero base value.

Clause 24. The system of any of clauses 1-23, wherein the instructions, when executed by the one or more processors, further cause the system to:
identify a first potential end point of the first glucose episode;
calculate an integrated area under the curve over time of a portion of the graph from the first start point to the first potential end point;
assign a total count value for the integrated area under the curve over time of the portion of the graph from the first start point to the first potential end point; and
confirm the first potential end point as a first end point of the first glucose episode if the first potential end point satisfies at least one end point condition.

Clause 25. The system of clause 24, wherein the at least one end point is confirmed if the total count value is less than a threshold count value.

Clause 26. The system of clause 24, wherein the first potential end point is confirmed as the first end point if a difference between a glucose level of the first start point of the first glucose episode and a glucose level of the first potential end point is below a threshold difference.

Clause 27. The system of clause 24, wherein the first potential end point is confirmed as the first end point if the first potential end point is a local minimum as compared to a previous adjacent data point.

Clause 28. The system of clause 24, wherein the first potential end point is confirmed as the first end point if a calculated integrated area under the curve over time of a portion of the graph from the start point to the first potential end point is less than a minimum episode point threshold score.

Clause 29. The system of any of clauses 1-28, wherein the instructions, when executed by the one or more processors, further cause the system to:
identify, in the dataset, at least one potential local minimum in at least one additional time period, wherein the at least one additional time period comprises a beginning data point and a last received glucose data point in the at least one additional time period;
confirm at least one potential local minimum as a start point of at least one additional glucose episode if the at least one potential local minimum satisfies the at least one local minimum condition;
calculate an integrated area under the curve over time of at least one additional portion of the graph from the start point of the at least one additional glucose episode to the last received glucose data point in the at least one additional time period; and
assign at least one additional first count value for the at least one additional portion.

Clause 30. The system of clause 29, wherein the instructions, when executed by the one or more processors, further cause the system to:
identify at least one additional potential end point of the at least one additional glucose episode;
confirm the at least one additional potential end point as an end point of the at least one additional glucose episode if the at least one additional potential end point satisfies the at least one end point condition;
calculate an integrated area under the curve over time of the at least one additional portion of the graph from the start point of at least one additional glucose episode to the at least one additional potential end point; and
assign a total count value for the integrated area under the curve over time of the at least one additional portion of the graph from the start of at least one additional glucose episode to the at least one additional potential end point.

Clause 31. The system of clause 30, wherein the at least one end point is confirmed if the total count value for the integrated area under the curve over time of the at least one additional portion of the graph is less than threshold count value.

Clause 32. The system of any of clauses 1-31, wherein the wireless communications circuitry is further configured to receive input related to exercise, and wherein the instructions, when executed by the one or more processors, further cause the system to:
determine if the first glucose episode is associated with exercise; and
disregard the calculated integrated area under the curve over time for the first glucose episode.

Clause 33. The system of any of clauses 1-32, wherein the wireless communications circuitry is further configured to receive input related to exercise from a user, and wherein the instructions, when executed by the one or more processors, further cause the system to:
determine that the first glucose episode is flagged by a user to not assign a count value for the first glucose episode; and
disregard the calculated integrated area under the curve over time for the first glucose episode if the first glucose episode was flagged by the user.

Clause 34. A system for determining metrics relating to a user, the system comprising:
wireless communications circuitry configured to receive time-correlated measured glucose data;
a display configured to visually present information; and
one or more processors coupled with the wireless communications circuitry, the display, and a memory storing instructions that, when executed by the one or more processors, cause the system to:
determine a glucose metric for each of a plurality of glucose episodes in a dataset of time-correlated glucose data in a time period;
   assign a count value for each glucose episode of the plurality of glucose episodes based on a comparison of the determined glucose metric to a distribution of glucose metrics determined from a predetermined population;
   determine an aggregate count value for each of a plurality of time-of-day periods; and
   assign a glycemic profile from a plurality of glycemic profiles based on the determined aggregate count value for each of the plurality of time-of-day periods.

Clause 35. The system of clause 34, wherein the glucose metric comprises a calculated integrated areas under a curve over time of a graph of the dataset for each glucose episode of the plurality of glucose episodes.

Clause 36. The system of any of clauses 34-35, wherein the plurality of time-of-day periods comprises at least 3 time-of-day periods.

Clause 37. The system of any of clauses 34-36, wherein the plurality of time-of-day periods comprises a morning period, an afternoon period, an evening period, and an overnight period.

Clause 38. The system of any of clauses 34-37, wherein the aggregate count value for each of the plurality of time-of-day periods is determined by averaging a count total for each time-of-day period in the time period.

Clause 39. The system of any of clauses 34-38, wherein the aggregate count value for each of the plurality of time-of-day periods is determined by identifying a median count total for each time-of-day period in the time period.

Clause 40. The system of any of clauses 34-39, wherein the aggregate count value for each of the plurality of time-of-day periods is determined by determining a sum of a count total for each time-of-day period in the time period.

Clause 41. The system of any of clauses 34-40, wherein the time period is about 1 week.

Clause 42. The system of any of clauses 34-41, wherein the glycemic profile is assigned based on a determined time-of-day period having a highest aggregate count value.

Clause 43. The system of clause 42, wherein a first glycemic profile is assigned if the determined aggregate count value is highest in a morning time-of-day period.

Clause 44. The system of clause 42, wherein a second glycemic profile is assigned if the determined aggregate count value is highest in an afternoon time-of-day period.

Clause 45. The system of clause 42, wherein a third glycemic profile is assigned if the determined aggregate count value is highest in an evening time-of-day period.

Clause 46. The system of clause 42, wherein a fourth glycemic profile is assigned if the determined aggregate count value is highest in an overnight time-of-day period.

Clause 47. The system of clause 42, wherein a fifth glycemic profile is assigned if the determined aggregate count values for at least two time-of-day periods are equal.

Clause 48. The system of any of clauses 34-47, wherein the instructions, when executed by the one or more processors, further cause the system to:
output a recommendation based on the assigned glycemic profile.

Clause 49. The system of clause 48, wherein the recommendation is further based on at least one characteristic of a user selected from the group consisting of an age, a height, a weight, a BMI, a gender, a race, and an ethnicity.

Clause 50. The system of clause 48, wherein the recommendation is further based on at least one input logged by a user, the at least one input selected from the group consisting of food, stress, sleep, mood, and exercise.

Clause 51. The system of clause 48, wherein the recommendation is further based on a particular geographic location of a user.

Clause 52. The system of any of clauses 34-51, wherein the count value is assigned based on a population distribution of integrated areas under the curve linearized to a range of count values.

Clause 53. A system for monitoring metrics relating to a user, the system comprising:
wireless communications circuitry configured to receive time-correlated measured glucose data;
a display configured to visually present information; and
one or more processors coupled with the wireless communications circuitry, the display, and a memory storing instructions that, when executed by the one or more processors, cause the system to:
assign a count value for each glucose episode based at least on an area under a curve of the each glucose episode in a dataset of time-correlated glucose data;
   calculate a running sum of count values for a plurality of glucose episodes in a time period; and
   display a progress indicator representative of the running sum of the count values relative to a target count goal for the time period.

Clause 54. The system of clause 53, wherein the count value is assigned based on a comparison to a distribution of a glucose metric determined from a predetermined population.

Clause 55. The system of any of clauses 53-54, wherein the dataset comprises a graph of glucose data vs. time.

Clause 56. The system of any of clauses 53-55, wherein the time period is about one day.

Clause 57. The system of any of clauses 53-56, wherein the progress indicator comprises a display of a fraction comprising the running sum of count values in a numerator and the target count goal in a denominator.

Clause 58. The system of clause 57, wherein the display of the fraction has a first end, a second end, and a length, and wherein a numerical value of the running sum of count values is displayed at a position along the length of the numerator that is proportional to [the running sum of count values]/[the target count goal] if the running sum of count values is less than or equal to the target count goal.

Clause 59. The system of clause 58, wherein the target count goal is displayed in the denominator near the second end.

Clause 60. The system of clause 57, wherein the display of the fraction has a first end, a second end, and a length, and wherein a numerical value of the target count goal is displayed at a position along the length of the denominator that is proportional to [the target count goal]/[the running sum of count values] if the running sum of count values is greater than the target count goal.

Clause 61. The system of clause 60, wherein a numerical value of the running sum of count values is displayed at the first end.

Clause 62. The system of clause 58, wherein when the numerical value of the running sum of count values is zero, the numerical value of the running sum of count values is displayed near the first end.

Clause 63. The system of any of clauses 53-62, wherein the instructions, when executed by the one or more processors, further cause the system to:
determine a difference between a first count value assigned in a first time period and a second count value assigned in a second time period, wherein the second time period is immediately after the first time period;
   assign a count trend status for the second time period from a plurality of count trend statuses based on the determined difference; and
   display a color representative of the assigned count trend status for the second time period.

Clause 64. The system of clause 63, wherein the plurality of count trend statuses comprises a balanced status, a declining in spike status, a flat during spike status, and a rising in spike status.

Clause 65. The system of clause 63, wherein the first time period and the second time period both occur during a single glucose episode.

Clause 66. The system of clause 63, wherein the progress indicator is displayed in a graphic user interface (GUI), and wherein the color is displayed as a background color of the GUI.

Clause 67. The system of clause 63, wherein the color representative of the assigned count trend status is displayed as a background color of a graphic user interface (GUI).

Clause 68. The system of clause 63, wherein the instructions, when executed by the one or more processors, further cause the system to:
determine a difference between at least one additional count value in at least one additional time period and the second count value, wherein the at least one additional time period is immediately after the second time period;
   assign a count trend status for the at least one additional time period from a plurality of count trend statuses based on the determined difference; and
   display a color representative of the assigned count trend status for the at least one additional time period.

Clause 69. The system of clause 68, wherein the display comprises a graphic user interface, and wherein the color representative of the assigned count trend status for the at least one additional time period is displayed as a background color of a graphic user interface (GUI).

Clause 70. The system of clause 69, wherein the color representative of the assigned count trend status for the at least one additional time period is displayed as a background color of a first portion of the GUI, and the color representative of the assigned count trend status for the second time period is displayed in a second portion of the GUI.

Clause 71. The system of clause 70, wherein the first portion of the GUI is a top portion and wherein the second portion of the GUI is a bottom portion.

Clause 72. The system of clause 70, wherein the color representative of the assigned count trend status for the at least one additional time period and the color representative of the assigned count trend status for the second time period are displayed as blended colors.

Clause 73. The system of clause 70, wherein the color representative of the assigned count trend status for the at least one additional time period and the color representative of the assigned count trend status for the second time period are displayed in a gradient.

Clause 74. The system of any of clauses 53-73, wherein the instructions, when executed by the one or more processors, further cause the system to:
determine a slope for a line formed by count values determined for a plurality of periods of time;
   assign a count trend status for at least one of the plurality of periods of time based on the determined slope; and
   display a color representative of the assigned count trend status for the second time period.

Clause 75. The system of clause 74, wherein the plurality of count trend statuses comprises a balanced status, a declining in spike status, a flat during spike status, and a rising in spike status.

Clause 76. The system of clause 74, wherein the count trend status is rising in spike if the slope is positive.

Clause 77. The system of clause 74, wherein the count trend status is declining in spike if the slope is negative.

Clause 78. The system of clause 74, wherein the count trend status is flat in spike if the slope is substantially constant.

Clause 79. The system of clause 74, wherein the plurality of periods of time are consecutive.

Clause 80. A system for monitoring metrics relating to a user, the system comprising:
wireless communications circuitry configured to receive time-correlated measured glucose data;
a display configured to visually present information; and
one or more processors coupled with the wireless communications circuitry, the display, and a memory storing instructions that, when executed by the one or more processors, cause the system to:
assign a count for each glucose episode based at least on an area under a curve of the each glucose episode in a dataset of time-correlated glucose data;
   determine a difference between a first count value assigned to a first glucose episode in a first time period and a second count value assigned to a second glucose episode in a second time period, wherein the second time period is immediately after the first time period; and
   assign a count trend status for the second time period based on the determined difference between the first count value and the second count value, wherein the count trend status is one of a plurality of count trend statuses.

Clause 81. The system of clause 80, wherein the instructions, when executed by the one or more processors, further cause the system to:
display a color representative of the assigned count trend status for the second time period.

Clause 82. The system of any of clauses 80-81, wherein the plurality of count trend statuses comprises a balanced status, a declining in spike status, a flat during spike status, and a rising in spike status.

Clause 83. The system of any of clauses 80-82, wherein the first time period and the second time period both occur during a single glucose episode.

Clause 84. A system for monitoring metrics relating to a user, the system comprising:
wireless communications circuitry configured to receive time-correlated measured glucose data;
a display configured to visually present information; and
one or more processors coupled with the wireless communications circuitry, the display, and a memory storing instructions that, when executed by the one or more processors, cause the system to:
assign a first count value for a glucose episode in a first time period and a second count value for a glucose episode in a second time period, wherein each of the first and second count values are based at least on an area under a curve of the each glucose episode in a dataset of time-correlated glucose data;
   determine a slope for a line formed by the first count value and the second count value; and
   assign a count trend status for one of the first or second time periods based on the determined slope.

Clause 85. The system of clause 84, wherein the instructions, when executed by the one or more processors, further cause the system to:
display a color representative of the assigned count trend status for the second time period.

Clause 86. The system of any of clauses 84-85, wherein the count trend status is rising in spike if the slope is positive.

Clause 87. The system of any of clauses 84-86, wherein the count trend status is declining in spike if the slope is negative.

Clause 88. The system of any of clauses 84-87, wherein the count trend status is flat in spike if the slope is substantially constant.

Clause 89. The system of any of clauses 84-88, wherein the glucose episode in the first time period and the glucose episode in the second time period are parts of a single glucose episode.

Clause 90. The system of any of clauses 84-89, wherein the glucose episode in the first time period and the glucose episode in the second time period are different glucose episodes.

Clause 91. A system for monitoring metrics relating to a user, the system comprising:
wireless communications circuitry configured to receive time-correlated measured glucose data;
a display configured to visually present information; and
one or more processors coupled with the wireless communications circuitry, the display, and a memory storing instructions that, when executed by the one or more processors, cause the system to:
assign a count value for each glucose episode based at least on an area under a curve of the each glucose episode in a dataset of time-correlated glucose data;
   display a summary graphic comprising a total count value for each of a plurality of time-of-day periods for a day, wherein the total count value for each of the plurality of time-of-day periods is a sum of the count values for each plurality of glucose episodes occurring during each of the plurality of time-of-day periods; and
   display a total count value for the day relative to a target count goal for the day.

Clause 92. The system of clause 91, wherein the summary graphic comprises four portions corresponding to four time-of-day periods, each portion comprising a numerical display of the total count value for one of the four time-of-day periods.

Clause 93. The system of any of clauses 91-92, wherein a portion corresponding to a highest total count value is a different color than a rest of the four portions.

Clause 94. The system of any of clauses 91-92, wherein the summary graphic is a pie chart.

Clause 95. The system of any of clauses 91-92, wherein the summary graphic is bar graph.

Clause 96. The system of any of clauses 91-92, wherein the summary graphic is circular graphic.

Clause 97. The system of any of clauses 91-92, wherein the display of the total count value for the day relative to the target count goal for the day is located in a center of the summary graphic.

Clause 98. The system of any of clauses 91-97, wherein the instructions, when executed by the one or more processors, further cause the system to display text identifying a time-of-day period having a highest total count value.

Clause 99. The system of any of clauses 91-98, wherein the instructions, when executed by the one or more processors, further cause the system to display a recommendation based on the total count value for the day.

Clause 100. The system of any of clauses 91-99, wherein the instructions, when executed by the one or more processors, further cause the system to display a recommendation based on the determined time-of-day period having a highest total count value.

Clause 101. The system of any of clauses 91-100, wherein the instructions, when executed by the one or more processors, further cause the system to display a list of untagged events from the day.

Clause 102. The system of any of clauses 91-101, wherein the instructions, when executed by the one or more processors, further cause the system to display a prompt for a user to tag events detected during the day.

Clause 103. The system of any of clauses 91-103, wherein the instructions, when executed by the one or more processors, further cause the system to display recommendations related to prioritizing protein.

Clause 104. A system for monitoring metrics relating to a user, the system comprising:
wireless communications circuitry configured to receive time-correlated measured glucose data;
a display configured to visually present information; and
one or more processors coupled with the wireless communications circuitry, the display, and a memory storing instructions that, when executed by the one or more processors, cause the system to:
assign a count value for each glucose episode in a dataset of time-correlated glucose data based at least on an area under a curve of the each glucose episode;
   determine an aggregate total daily count value during a time period;
   determine an aggregate count value for each of a plurality of time-of-day periods during the time period; and
   display a summary graphic comprising the aggregate count value for each of the plurality of time-of-day periods, the aggregate total daily count value during the time period, and a target count goal for the day.

Clause 105. The system of clause 104, wherein the count value is assigned based on a comparison to a distribution of a glucose metric determined from a predetermined population.

Clause 106. The system of any of clauses 104-105, wherein the time period is one week.

Clause 107. The system of any of clauses 104-106, wherein the aggregate total daily count value is an average total daily count value, wherein the aggregate count value for each of a plurality of time-of-day periods is an average count value for each of a plurality of time-of-day periods.

Clause 108. The system of any of clauses 104-107, wherein the summary graphic is a circular graphic, and wherein the aggregate total daily count value during the time period and the target count goal for the day are displayed in a center of the circular graphic.

Clause 109. The system of any of clauses 104-108, wherein each of the aggregate count values for each of a plurality of time-of-day periods during the time period are displayed in a different portion of the circular graphic.

Clause 110. The system of clause 108, wherein a portion of the circular graphic corresponding to a time-of-day period with a highest aggregate count value is a different color than a rest of the circular graphic.

Clause 111. The system of clause 108, wherein the time-of-day periods are arranged clockwise in the circular graphic.

Clause 112. The system of any of clauses 104-111, wherein the summary graphic is a bar graph, and wherein bars corresponding to days having an aggregate total daily count higher than the target count goal comprise a first color and bars corresponding to days having an aggregate total daily count lower than or equal to the target count goal comprise a second color.

Clause 113. The system of any of clauses 104-112, wherein the instructions, when executed by the one or more processors, further cause the system to:
display a summary for each day of the time period.

Clause 114. The system of clause 113, wherein the summary for each day comprises a daily count total and a graphic highlighting a time-of-day period with a highest count value.

Clause 115. The system of any of clauses 104-114, wherein the instructions, when executed by the one or more processors, further cause the system to:
display a comparison of the determined aggregate total daily count value to a plurality of total daily count values for a population.

Clause 116. The system of clause 115, wherein the population is related to an age of a user.

Clause 117. The system of any of clauses 104-116, wherein the instructions, when executed by the one or more processors, further cause the system to:
display a recommended time-of-day period for a user to mitigate.

Clause 118. The system of any of clauses 104-117, wherein the instructions, when executed by the one or more processors, further cause the system to:
determine a new target count goal based on a sum of the assigned count value for each glucose episode in the time period; and
display the new target count goal.

Clause 119. The system of any of clauses 104-118, wherein the instructions, when executed by the one or more processors, further cause the system to:
assign a glycemic profile from a plurality of glycemic profiles based on the determined aggregate count value for each of the plurality of time of day periods; and
display the assigned glycemic profile.

Clause 120. The system of any of clauses 104-119, wherein the time period is one week, and wherein the instructions, when executed by the one or more processors, further cause the system to:
display a plurality of graphic elements corresponding to each day of the time period, wherein each of the plurality of graphic elements comprises the aggregate total daily count value determined for each day of the time period.

Clause 121. The system of clause 120, wherein a graphic element of the plurality of graphic elements having an aggregate total daily count value equal to or below the target count goal for the day is visually distinguishable from a graphic element of the plurality of graphic elements having an aggregate total daily count value above the target count goal for the day.

Clause 122. The system of any of clauses 104-121, wherein the instructions, when executed by the one or more processors, further cause the system to:
display a graph comprising the determined aggregate total daily count value during the time period and at least one aggregate daily count value determined from an earlier time period.

Clause 123. The system of any of clauses 104-122, wherein the instructions, when executed by the one or more processors, further cause the system to:
display a focus area identified by the user.

Clause 124. The system of any of clauses 104-123, wherein the wireless communications circuitry is configured to receive data related to a focus area identified by the user, and wherein the instructions, when executed by the one or more processors, further cause the system to:
display a graph of answers from a user prompt related to the focus area identified by the user.

Clause 125. The system of clause 124, wherein the graph is a bar graph.

Clause 126. The system of any of clauses 104-125, wherein the instructions, when executed by the one or more processors, further cause the system to:
display a graphic comprising the target count goal for the day for the time period and at least one target count goal for the day for a previous time period.

Clause 127. A system for monitoring metrics relating to a user, the system comprising:
wireless communications circuitry configured to receive measured glucose data;
a display configured to visually present information; and
one or more processors coupled with the wireless communications circuitry, the display, and a memory storing instructions that, when executed by the one or more processors, cause the system to:
identify, in a dataset of time correlated glucose data, a first potential local minimum in a first time period, wherein the first time period comprises a beginning data point and a last received glucose data point in the first time period;
confirm the first potential local minimum as a first start point of a first glucose episode if the first potential local minimum satisfies at least one local minimum condition;
calculate an integrated area under a curve over time of a first portion of a graph of the dataset beginning at the first start point of the first glucose episode to the last received glucose data point; and
   assign a first count value for the first portion.

Clause 128. The system of clause 127, wherein the beginning data point is a determined end point of a previous adjacent glucose episode.

Clause 129. The system of any of clauses 127-128, wherein the beginning data point has a timestamp that is between about 60 to about 90 minutes before a timestamp of the last received glucose data point.

Clause 130. The system of any of clauses 127-129, wherein the first count value is assigned based on a comparison to a distribution of a glucose metric determined from a predetermined population.

Clause 131. The system of any of clauses 127-130, wherein the dataset is a graph of glucose vs. time.

Clause 132. The system of any of clauses 127-131, wherein the first potential local minimum is confirmed as the first start point of the first glucose episode if a rate of change between the first potential local minimum and a previous point within the first time period is above a rate of change threshold value.

Clause 133. The system of clause 132, wherein the previous point is within about 20 minutes of the first potential local minimum.

Clause 134. The system of clause 132, wherein the previous point is a previous closest local minimum.

Clause 135. The system of any of clauses 127-134, wherein the instructions, when executed by the one or more processors, further cause the system to:
display the first count value.

Clause 136. The system of any of clauses 127-135, wherein the instructions, when executed by the one or more processors, further cause the system to:
identify, in the dataset, at least one additional potential local minimum in the first time period; and
confirm the at least one additional potential local minimum as the first start point if the at least one additional potential local minimum satisfies the at least one local minimum condition.

Clause 137. The system of clause 136, wherein the step of identifying the at least one additional potential local minimum in the first time period is performed after the first potential local minimum is confirmed as a start point if the first potential local minimum satisfies the at least one local minimum condition.

Clause 138. The system of any of clauses 127-137, wherein the instructions, when executed by the one or more processors, further cause the system to:
identify, in the dataset, a first alert point for a potential glucose episode if the last received glucose data point in the first time period satisfies at least one alert condition.

Clause 139. The system of clause 138, wherein the at least one alert condition comprises confirming that a calculated rate of change between the first alert point and a previous point within about 20 minutes of the first alert point is above an alert rate of change threshold.

Clause 140. The system of clause 138, wherein the at least one alert condition comprises confirming that a difference between the first alert point and the first potential local minimum is above a local minimum alert threshold.

Clause 141. The system of clause 138, wherein the at least one alert condition comprises confirming that a calculated integrated area under the curve from the first potential local minimum to the alert point corresponds to a count value above a threshold count value.

Clause 142. The system of clause 138, wherein the at least one local minimum condition comprises determining if a difference between a glucose level of the first alert point and a glucose level of the first potential local minimum is above a threshold difference.

Clause 143. The system of any of clauses 127-142, wherein the instructions, when executed by the one or more processors, further cause the system to:
confirm that a last received glucose data point in a second time period is part of the first glucose episode;
calculate an integrated area under the curve over time of a second portion of the graph beginning at the first start point to the last received glucose data point in the second time period; and
assign a second count value for the second portion.

Clause 144. The system of clause 143, wherein the instructions, when executed by the one or more processors, further cause the system to:
display the second count value.

Clause 145. The system of any of clauses 127-137, wherein the instructions, when executed by the one or more processors, further cause the system to:
identify a first potential end point of the first glucose episode;
calculate an integrated area under the curve over time of a portion of the graph from the first start point to the first potential end point;
assign a total count value for the integrated area under the curve over time of the portion of the graph from the first start point to the first potential end point; and
confirm the first potential end point as a first end point of the first glucose episode if the first potential end point satisfies at least one end point condition.

Clause 146. The system of clause 145, wherein the at least one end point is confirmed if the total count value is less than a threshold count value.

Clause 147. The system of clause 145, wherein the first potential end point is confirmed as the first end point if a difference between a glucose level of the first start point of the first glucose episode and a glucose level of the first potential end point is below a threshold difference.

Clause 148. The system of clause 145, wherein the first potential end point is confirmed as the first end point if the first potential end point is a local minimum as compared to a previous adjacent data point.

Clause 149. The system of clause 145, wherein the first potential end point is confirmed as the first end point if a calculated integrated area under the curve over time of a portion of the graph from the start point to the first potential end point is less than a minimum episode point threshold score.

Clause 150. The system of any of clauses 127-137, wherein the instructions, when executed by the one or more processors, further cause the system to:
identify, in the graph of glucose data vs. time, at least one potential local minimum in at least one additional time period, wherein the at least one additional time period comprises a beginning data point and a last received glucose data point in the at least one additional time period;
confirm at least one potential local minimum as a start point of at least one additional glucose episode if the at least one potential local minimum satisfies the at least one local minimum condition;
calculate an integrated area under the curve over time of at least one additional portion of the graph from the start point of the at least one additional glucose episode to the last received glucose data point in the at least one additional time period; and
assign at least one additional first count value for the at least one additional portion.

Clause 151. The system of clause 150, wherein the instructions, when executed by the one or more processors, further cause the system to:
identify at least one additional potential end point of the at least one additional glucose episode;
confirm the at least one additional potential end point as an end point of the at least one additional glucose episode if the at least one additional potential end point satisfies the at least one end point condition;
calculate an integrated area under the curve over time of the at least one additional portion of the graph from the start point of at least one additional glucose episode to the at least one additional potential end point; and
assign a total count value for the integrated area under the curve over time of the at least one additional portion of the graph from the start of at least one additional glucose episode to the at least one additional potential end point.

Clause 152. The system of clause 151, wherein the at least one end point is confirmed if the total count value for the integrated area under the curve over time of the at least one additional portion of the graph is less than threshold count value.

Clause 153. The system of any of clauses 127-152, wherein the wireless communications circuitry is further configured to receive input related to exercise, and wherein the instructions, when executed by the one or more processors, further cause the system to:
determine that the first glucose episode is associated with exercise; and
disregard the calculated integrated area under the curve over time for the first glucose episode.

Clause 154. The system of any of clauses 127-153, wherein the wireless communications circuitry is further configured to receive input related to exercise from a user, and wherein the instructions, when executed by the one or more processors, further cause the system to:
determine if the first glucose episode is flagged by a user; and
disregard the calculated integrated area under the curve over time for the first glucose episode if the first glucose episode was flagged by a user.

Clause 155. A system for determining metrics relating to a user, the system comprising:
wireless communications circuitry configured to receive measured glucose data;
a display configured to visually present information; and
one or more processors coupled with the wireless communications circuitry, the display, and a memory storing instructions that, when executed by the one or more processors, cause the system to:
assign a count value for each glucose episode of a plurality of glucose episodes in a dataset of time-correlated glucose data based at least on an area under the curve of the each glucose episode;
   categorize each of the plurality of glucose episodes as a food event or a non-food event; and
   calculate a first running sum of count values for each glucose episode categorized as a food event.

Clause 156. The system of clause 155, wherein the instructions, when executed by the one or more processors, further cause the system to:
calculate a second running sum of count values for each glucose episode categorized as a non-food event.

Clause 157. The system of any of clauses 155-156, wherein the instructions, when executed by the one or more processors, further cause the system to:
output the second running sum of count values on the display.

Clause 158. The system of any of clauses 155-157, wherein the instructions, when executed by the one or more processors, further cause the system to:
output the first running sum of count values on the display.

Clause 159. The system of any of clauses 155-158, wherein the count value for each glucose episode is assigned based on a calculated integrated area under the curve over time for each glucose episode.

Clause 160. A system for determining metrics relating to a user, the system comprising:
wireless communications circuitry configured to receive measured glucose data;
a display configured to visually present information; and
one or more processors coupled with the wireless communications circuitry, the display, and a memory storing instructions that, when executed by the one or more processors, cause the system to:
determine an area under a curve for each glucose episode of a plurality of glucose episodes in a dataset of time-correlated glucose data;
   assign a count value for each glucose episode of the plurality of glucose episodes based on a comparison of the determined area under the curve to a distribution of areas under the curve determined from a predetermined population;
   determine an aggregate daily total count value for a first time period based on the assigned count value for each glucose episode of the plurality of glucose episodes; and
   determine a target daily count goal for a user for a second time period based on the determined aggregate daily total count value for the first time period.

Clause 161. The system of clause 160, wherein the target daily count goal for the user for the second time period is determined based on a comparison of the determined aggregate daily total count value for the first time period to a population of users having an aggregate daily total count value within a threshold of the determined aggregate daily total count value of the user.

Clause 162. The system of any of clauses 160-161, wherein the first time period comprises a first week.

Clause 163. The system of clause 162, wherein the second time period comprises a second week, wherein the second week occurs after the first week.

Clause 164. The system of any of clauses 162-163, wherein the first week and second week are consecutive.

Clause 165. A system for monitor in metrics relating to a user, the system comprising:
wireless communications circuitry configured to receive measured glucose data;
a display configured to visually present information; and
one or more processors coupled with the wireless communications circuitry, the display, and a memory storing instructions that, when executed by the one or more processors, cause the system to:
identify, in a dataset of time-correlated glucose data, a plurality of local maxima as potential peaks and a plurality of local minima as potential troughs in a time period;
screen the potential peaks and the potential troughs to determine a plurality of glucose episodes that satisfy at least one condition;
calculate an integrated area under a curve of the dataset over time for each of the plurality of glucose episodes; and
assign a count value to each of the plurality of glucose episodes.

Clause 166. The system of clause 165, wherein the plurality of local maxima and the plurality of local minima are screened by applying an algorithm.

Clause 167. The system of any of clauses 165-166, wherein the dataset of time-correlated glucose data comprises a graph of glucose data vs. time.

Clause 168. The system of clause 166, wherein the algorithm detects an episode in the plurality of glucose episodes if a rate of change between an identified peak and an identified trough is greater than a threshold rate of change.

Clause 169. The system of clause 166, wherein the algorithm detects an episode in the plurality of glucose episodes if a time difference between an identified peak and an identified trough is greater than a threshold time difference.

Clause 170. A system for monitoring metrics relating to a user, the system comprising:
wireless communications circuitry configured to receive time-correlated measured glucose data;
a display configured to visually present information; and
one or more processors coupled with the wireless communications circuitry, the display, and a memory storing instructions that, when executed by the one or more processors, cause the system to:
determine a start point of a glucose episode in a dataset of time-correlated glucose data;
   assign a first count value for a first portion of a glucose episode based at least on an area under a curve of the dataset over time, wherein the first portion of the glucose episode begins at the start point and extends to a last received glucose data point in a first time period;
   assign a second count value for a second portion of the glucose episode based at least on an area under the curve, wherein the second portion of the glucose episode begins at the last received glucose data point in the first time period and extends to a last received glucose data point in a second time period, wherein the second time period is immediately after the first time period;
   determine a difference between the first count value and the second count value;
   assign a count trend status for the second time period from a plurality of count trend statuses based on the determined difference; and
   display a graph of glucose data vs. time, wherein a portion of the graph corresponding to the second time period is displayed in a color representative of the assigned count trend status.

Clause 171. The system of clause 170, wherein the dataset comprises a graph of glucose data vs. time.

Clause 172. The system of any of clauses 170-171, wherein the instructions, when executed by the one or more processors, further cause the system to:
assign at least one additional count value for at least one additional portion of the glucose episode based at least on an area under the curve, wherein the at least one additional portion of the graph beginning at the last received glucose data point in the at least one additional time period to the last received glucose data point in the at least one additional time period, wherein the at least one additional time period is immediately after the second time period;
   determine a difference between the at least one additional count value and the second count value;
   assign a count trend status for the at least one additional time period from a plurality of count trend statuses based on the determined difference; and
   display the graph of glucose data vs. time, wherein a portion of the graph corresponding to the at least one additional time period is displayed in a color representative of the assigned count trend status for the at least one additional time period.

Clause 173. The system of clause 172, wherein at least a portion of an area under the curve of the graph of glucose data vs. time for the second time period comprises the color representative of the assigned count trend status for the second time period.

Clause 174. The system of any of clauses 170-173, wherein the first count value and the second count value are each assigned based on a comparison to a distribution of areas under the curve determined from a predetermined population.

Clause 175. The system of any of clauses 170-174, wherein the instructions, when executed by the one or more processors, further cause the system to:
calculate a running count value for the glucose episode; and
display the running count value near the glucose episode in the graph of glucose data vs. time.

Clause 176. The system of any of clauses 170-175, wherein the instructions, when executed by the one or more processors, further cause the system to:
calculate a total count value for the glucose episode; and
display the total count value near the glucose episode in the graph of glucose data vs. time.

Clause 177. The system of any of clauses 170-176, wherein a y-axis of the graph of glucose data vs. time represents glucose levels.

Clause 178. The system of clause 177, wherein the y-axis is not labeled with numerical values.

Clause 179. The system of any of clauses 170-178, wherein the instructions, when executed by the one or more processors, further cause the system to:

display a numerical value of a glucose level in the graph of glucose data vs. time in response to a user scrolling through the graph.

Clause 180. The system of any of clauses 170-179, wherein the instructions, when executed by the one or more processors, further cause the system to:

display a numerical value of a glucose level in the graph of glucose data vs. time in response to a user applying pressure to a point on the graph.

Clause 181. The system of any of clauses 170-181, wherein the wireless communications circuitry is further configured to receive time-correlated logged data, and wherein the instructions, when executed by the one or more processors, further cause the system to:
display an icon related to logged data on the graph of glucose data vs. time near a time associated with the logged data.

Clause 182. The system of clause 181, wherein the logged data comprises lifestyle events, activity events, food, or combinations thereof.

Clause 183. The system of any of clauses 170-182, wherein the time-correlated measured glucose data is received about every 5 minutes.

Clause 184. The system of any of clauses 170-183, wherein the time-correlated measured glucose data is received about every 15 minutes.

Clause 185. A system for monitoring metrics relating to glucose management of a user, the system comprising:
wireless communications circuitry configured to receive time-correlated measured glucose data and data related answers from at least one prompt to a user;
a display configured to visually present information; and
one or more processors coupled with the wireless communications circuitry, the display, and a memory storing instructions that, when executed by the one or more processors, cause the system to:
determine a daily target count goal for a time period;
   assign a count value for each glucose episode based at least on an area under a curve of the each glucose episode in a dataset of time-correlated glucose data in the time period;
   determine a total count value for each of a plurality of days of the time period; and
   display a plurality of graphic elements corresponding to each day of the time period, wherein each of the plurality of graphic elements comprises the total count value determined for each of the plurality of days of the time period.

Clause 186. The system of clause 185, wherein the count value is assigned based on a comparison to a distribution of areas under the curve determined from a predetermined population.

Clause 187. The system of any of clauses 185-186, wherein the time period is one week.

Clause 188. The system of any of clauses 185-187, wherein a graphic element of the plurality of graphic elements corresponding to a first day of the time period comprises a total daily count value determined for the first day.

Clause 189. The system of any of clauses 185-188, wherein a graphic element of the plurality of graphic elements having a total daily count value equal to or below the daily target count goal is visually distinguishable from a graphic element of the plurality of graphic elements having a total daily count value above the daily target count goal.

Clause 190. The system of any of clauses 185-189, wherein the instructions, when executed by the one or more processors, further cause the system to:
display an indication of a focus area for the user.

Clause 191. The system of clause 190, wherein the focus area is selected from the group consisting of boost energy, manage hunger, improve mood, better sleep, and stay focused.

Clause 192. The system of any of clauses 185-191, wherein the daily target count goal is determined based on a comparison to a distribution of the counts determined for a predetermined population.

Clause 193. The system of clause 192, wherein the predetermined population is determined according to an age of the user.

Clause 194. The system of any of clauses 185-193, wherein the daily target count goal is determined based on a total count value determined for at least one day of a previous time period.

Clause 195. The system of any of clauses 185-194, wherein the instructions, when executed by the one or more processors, further cause the system to:
display a recommended time-of-day period for a user to mitigate.

Clause 196. The system of any of clauses 185-195, wherein the instructions, when executed by the one or more processors, further cause the system to:
determine an aggregate total daily count value for the time period and an aggregate total daily count value for at least one previous time period; and
display a graph comprising the aggregate total daily count value for the time period and the aggregate total daily count value for the at least one previous time period.

Clause 197. The system of clause 196, wherein the aggregate total daily count value for the time period comprises the average total daily count value for the time period, and wherein the aggregate total daily count value for the at least one previous time period comprises the average total daily count value for the at least one previous time period.

Clause 198. The system of any of clauses 185-197, wherein the glucose management comprises at least a first stage and a second stage, wherein advancement of the user from the first stage to the second stage requires the user to satisfy at least one requirement, wherein the instructions, when executed by the one or more processors, further cause the system to:
display at least one progress indicator relating to the satisfaction of the at least one requirement.

Clause 199. The system of clause 198, wherein the at least one requirement comprises the user having a total daily count value equal to or below the daily target count goal for a minimum number of days in the time period.

Clause 200. The system of clause 199, wherein the minimum number of days is about 5 days and the time period is about one week.

Clause 201. The system of clause 200, wherein the at least one requirement further comprises the user having a total daily count value equal to or below the daily target count goal for about 5 days in a one week time period for at least two weeks.

Clause 202. A system for determining metrics relating to a subject, the system comprising:
an input configured to receive measured glucose data;
a display configured to visually present information; and
one or more processors coupled with the input, the display, and a memory storing instructions that, when executed by the one or more processors, cause the system to:
determine a plurality of glucose metrics for each of a plurality of glucose spikes in a graph of glucose data vs. time over a time period;
   assign a count value for each glucose metric of the plurality of glucose metrics based on a comparison to a distribution of the glucose metric determined from a predetermined population;
   determine an aggregate count value for each of a plurality of time-of-day periods during the time period; and
   assign a glycemic profile from a plurality of glycemic profiles based on the determined aggregate count value for each of the plurality of time-of-day periods.

Clause 203. The system of clause 202, wherein the plurality of glucose metrics comprises a plurality of calculated integrated areas under the curve for each of the plurality of glucose spikes.

Clause 204. The system of any of clauses 202-203, wherein the plurality of time-of-day periods comprises at least 3 time of day periods.

Clause 205. The system of any of clauses 202-204, wherein the plurality of time-of-day periods comprises a morning period, an afternoon period, an evening period, and an overnight period.

Clause 206. The system of any of clauses 202-205, wherein the aggregate count value for each of the plurality of time-of-day periods is determined by averaging a count total for each time-of-day period in the time period.

Clause 207. The system of any of clauses 202-206, wherein the aggregate count value for each of the plurality of time-of-day periods is determined by identifying a median count total for each time-of-day period in the time period.

Clause 208. The system of any of clauses 202-207, wherein the aggregate count value for each of the plurality of time-of-day periods is determined by determining a sum of a count total for each time-of-day period in the time period.

Clause 209. The system of any of clauses 202-208, wherein the time period is about 1 week.

Clause 210. The system of any of clauses 202-209, wherein the glycemic profile is assigned based on a determined time-of-day period having a highest aggregate count value.

Clause 211. The system of clause 210, wherein a first glycemic profile is assigned if the determined aggregate count value is highest in a morning time-of-day period.

Clause 212. The system of any of clauses 210-211, wherein a second glycemic profile is assigned if the determined aggregate count value is highest in an afternoon time-of-day period.

Clause 213. The system of any of clauses 210-212, wherein a third glycemic profile is assigned if the determined aggregate count value is highest in an evening time-of-day period.

Clause 214. The system of any of clauses 210-213, wherein a fourth glycemic profile is assigned if the determined aggregate count value is highest in an overnight time-of-day period.

Clause 215. The system of any of clauses 210-214, wherein a fifth glycemic profile is assigned if the determined aggregate count values for at least two time-of-day periods are equal.

Clause 216. The system of any of clauses 202-215, wherein the instructions, when executed by the one or more processors, further cause the system to:
output a recommendation based on the assigned glycemic profile.

Clause 217. The system of clause 216, wherein the recommendation is further based on at least one characteristic of a user selected from the group consisting of an age, a height, a weight, a BMI, a gender, a race, and an ethnicity.

Clause 218. The system of any of clauses 216-217, wherein the recommendation is further based on at least one input logged by a user, the at least one input selected from the group consisting of food, stress, sleep, mood, and exercise.

Clause 219. The system of any of clauses 216-218, wherein the recommendation is further based on a particular geographic location of a user.

Clause 220. The system of any of clauses 202-219, wherein the count value is assigned based on a population distribution of integrated areas under the curve linearized to a range of count values.

Clause 221. A system for monitoring and/or displaying metrics relating to a subject, the system comprising:
an input configured to receive time-correlated measured glucose data;
a display configured to visually present information; and
one or more processors coupled with the input, the display, and a memory storing instructions that, when executed by the one or more processors, cause the system to:
determine a plurality of glucose metrics for at least one glucose spike in a dataset of time-correlated glucose data;
   assign a count value for each glucose metric of the plurality of glucose metrics;
   calculate a running sum of the count values for each glucose metric assigned in a time period; and
   display a progress indicator representative of the running sum of the count values relative to a total count goal for the time period.

Clause 222. The system of clause 221, wherein the count value is assigned based on a comparison to a distribution of the glucose metric determined from a predetermined population.

Clause 223. The system of any of clauses 221-222, wherein the dataset comprises a graph of glucose data vs. time.

Clause 224. The system of any of clauses 221-223, wherein the time period is about one day.

Clause 225. The system of any of clauses 221-224, wherein the plurality of glucose metrics are determined for a single glucose spike.

Clause 226. The system of any of clauses 221-225, wherein the plurality of glucose metrics are determined for a plurality of glucose spikes.

Clause 227. The system of any of clauses 221-226, wherein the progress indicator comprises a display of a fraction comprising the running sum of count values in a numerator and the total count goal in a denominator.

Clause 228. The system of clause 227, wherein the display of the fraction has a first end, a second end, and a length, and wherein a numerical value of the running sum of count values is displayed at a position along the length of the numerator that is proportional to [the running sum of count values]/[the total count goal] if the running sum of count values is less than or equal to the total count goal.

Clause 229. The system of clause 228, wherein the total count goal is displayed in the denominator near the second end.

Clause 230. The system of clause 227, wherein the display of the fraction has a first end, a second end, and a length, and wherein a numerical value of the total count goal is displayed at a position along the length of the denominator that is proportional to [the total count goal]/[the running sum of count values] if the running sum of count values is greater than the total count goal.

Clause 231. The system of clause 230, wherein a numerical value of the running sum of count values is displayed at the first end.

Clause 232. The system of any of clauses 228-231, wherein when the numerical value of the running sum of count values is zero, the numerical value of the running sum of count values is displayed near the first end.

Clause 233. The system of clause 221, wherein the instructions, when executed by the one or more processors, further cause the system to:
determine a difference between a first count value assigned in a first time period and a second count value assigned in a second time period, wherein the second time period is immediately after the first time period;
   assign a count trend status for the second time period from a plurality of count trend statuses based on the determined difference; and
   display a color representative of the assigned count trend status for the second time period.

Clause 234. The system of clause 233, wherein the plurality of count trend statuses comprises a balanced status, a declining in spike status, a flat during spike status, and a rising in spike status.

Clause 235. The system of any of clauses 233-234, wherein the first time period and the second time period both occur during a single glucose spike.

Clause 236. The system of any of clauses 233-235, wherein the progress indicator is displayed in a graphic user interface (GUI), and wherein the color is displayed as a background color of the GUI.

Clause 237. The system of any of clauses 233-236, wherein the color representative of the assigned count trend status is displayed as a background color of a graphic user interface (GUI).

Clause 238. The system of any of clauses 233-237, wherein the instructions, when executed by the one or more processors, further cause the system to:
determine a difference between the at least one additional count value and the second count value;
   assign a count trend status for the at least one additional time period from a plurality of count trend statuses based on the determined difference; and
   display a color representative of the assigned count trend status for the at least one additional time period.

Clause 239. The system of clause 238, wherein the color representative of the assigned count trend status for the at least one additional time period is displayed as a background color of a graphic user interface (GUI).

Clause 240. The system of clause 239, wherein the color representative of the assigned count trend status for the at least one additional time period is displayed as a background color of a first portion of the GUI, and the color representative of the assigned count trend status for the second time period is displayed in a second portion of the GUI.

Clause 241. The system of clause 240, wherein the first portion of the GUI is a top portion and wherein the second portion of the GUI is a bottom portion.

Clause 242. The system of any of clauses 240-241, wherein the color representative of the assigned count trend status for the at least one additional time period and the color representative of the assigned count trend status for the second time period are displayed as blended colors.

Clause 243. The system of any of clauses 240-242, wherein the color representative of the assigned count trend status for the at least one additional time period and the color representative of the assigned count trend status for the second time period are displayed in a gradient.

Clause 244. The system of any of clauses 221-243, wherein the instructions, when executed by the one or more processors, further cause the system to:
determine a slope for a line formed by count values determined for a plurality of periods of time;
   assign a count trend status for at least one of the plurality of periods of time based on the determined slope; and
   display a color representative of the assigned count trend status for the second time period.

Clause 245. The system of clause 244, wherein the plurality of count trend statuses comprises a balanced status, a declining in spike status, a flat during spike status, and a rising in spike status.

Clause 246. The system of clause 244, wherein the count trend status is rising in spike if the slope is positive.

Clause 247. The system of clause 244, wherein the count trend status is declining in spike if the slope is negative.

Clause 248. The system of clause 244, wherein the count trend status is flat in spike if the slope is substantially constant.

Clause 249. The system of clause 244, wherein the plurality of periods of time are consecutive.

Clause 250. A system for monitoring and/or displaying metrics relating to a subject, the system comprising:
an input configured to receive time-correlated measured glucose data;
a display configured to visually present information; and
one or more processors coupled with the input, the display, and a memory storing instructions that, when executed by the one or more processors, cause the system to:
determine a plurality of glucose metrics for at least one glucose spike in a graph of glucose data vs. time;
   assign a count value for each glucose metric of the plurality of glucose metrics;
   determine a difference between a first count value assigned in a first time period and a second count value assigned in a second time period, wherein the second time period is immediately after the first time period; and assign a count trend status for the second time period from a plurality of count trend statuses based on the determined difference.

Clause 251. The system of clause 250, wherein the instructions, when executed by the one or more processors, further cause the system to:
display a color representative of the assigned count trend status for the second time period.

Clause 252. The system of any of clauses 250-251, wherein the plurality of count trend statuses comprises a balanced status, a declining in spike status, a flat during spike status, and a rising in spike status.

Clause 253. The system of any of clauses 250-251, wherein the first time period and the second time period both occur during a single glucose spike.

Clause 254. A system for monitoring and/or displaying metrics relating to a subject, the system comprising:
an input configured to receive time-correlated measured glucose data;
a display configured to visually present information; and
one or more processors coupled with the input, the display, and a memory storing instructions that, when executed by the one or more processors, cause the system to:
determine a plurality of glucose metrics for at least one glucose spike in a graph of glucose data vs. time;
   assign a count value for each glucose metric of the plurality of glucose metrics;
   determine a slope for a line formed by count values determined for a plurality of periods of time; and
   assign a count trend status for at least one of the plurality of periods of time based on the determined slope.

Clause 255. The system of clause 254, wherein the instructions, when executed by the one or more processors, further cause the system to:
display a color representative of the assigned count trend status for the second time period.

Clause 256. The system of any of clauses 254-255, wherein the count trend status is rising in spike if the slope is positive.

Clause 257. The system of any of clauses 254-256, wherein the count trend status is declining in spike if the slope is negative.

Clause 258. The system of any of clauses 254-257, wherein the count trend status is flat in spike if the slope is substantially constant.

Clause 259. A system for monitoring and/or displaying metrics relating to a subject, the system comprising:
an input configured to receive time-correlated measured glucose data;
a display configured to visually present information; and
one or more processors coupled with the input, the display, and a memory storing instructions that, when executed by the one or more processors, cause the system to:
determine a plurality of glucose metrics for at least one glucose spike in a graph of glucose data vs. time;
   assign a count value for each glucose metric of the plurality of glucose metrics;
   display a summary graphic comprising a total count value for the glucose metric for each of a plurality of time-of-day periods for a day, wherein the total count value for each of the plurality of time-of-day periods is a sum of the count values for each glucose metric determined for a glucose spike occurring during each of the plurality of time-of-day periods; and
   display a total count value for the day relative to a total count goal for the day.

Clause 260. The system of clause 259, wherein the summary graphic comprises four portions corresponding to four time-of-day periods, each portion comprising a numerical display of the total count value for one of the four time-of-day periods.

Clause 261. The system of clause 260, wherein a portion corresponding to a highest total count value is a different color than a rest of the four portions.

Clause 262. The system of any of clauses 260-261, wherein the summary graphic is a pie chart.

Clause 263. The system of any of clauses 260-262, wherein the summary graphic is bar graph.

Clause 264. The system of any of clauses 260-263, wherein the summary graphic is circular graphic.

Clause 265. The system of any of clauses 260-264, wherein the display of the total count value for the day relative to the total count goal for the day is located in a center of the summary graphic.

Clause 266. The system of any of clauses 259-265, wherein the instructions, when executed by the one or more processors, further cause the system to display text identifying a time-of-day period having a highest total count value.

Clause 267. The system of any of clauses 259-266, wherein the instructions, when executed by the one or more processors, further cause the system to display a recommendation based on the total count value for the day.

Clause 268. The system of any of clauses 259-267, wherein the instructions, when executed by the one or more processors, further cause the system to display a recommendation based on the determined time-of-day period having a highest total count value.

Clause 269. The system of any of clauses 259-268, wherein the instructions, when executed by the one or more processors, further cause the system to display a list of untagged events from the day.

Clause 270. The system of any of clauses 259-269, wherein the instructions, when executed by the one or more processors, further cause the system to display a prompt for a user to tag events detected during the day.

Clause 271. The system of any of clauses 259-270, wherein the instructions, when executed by the one or more processors, further cause the system to display recommendations related to prioritizing protein.

Clause 272. A system for monitoring and/or displaying metrics relating to a subject, the system comprising:
an input configured to receive time-correlated measured glucose data;
a display configured to visually present information; and
one or more processors coupled with the input, the display, and a memory storing instructions that, when executed by the one or more processors, cause the system to:
determine a plurality of glucose metrics for each of a plurality of glucose spikes in a graph of glucose data vs. time in a time period;
   assign a count value for each glucose metric of the plurality of glucose metrics in the time period;
   determine an aggregate total daily count value during the time period;
   determine an aggregate count value for each of a plurality of time-of-day periods during the time period; and
   display a summary graphic comprising the aggregate count value for each of the plurality of time-of-day periods, the aggregate total daily count value during the time period, and a total count goal for the day.

Clause 273. The system of clause 272, wherein the count value is assigned based on a comparison to a distribution of the glucose metric determined from a predetermined population.

Clause 274. The system of any of clauses 272-273, wherein the time period is one week.

Clause 275. The system of any of clauses 272-274, wherein the aggregate total daily count value is an average total daily count value, wherein the aggregate count value for each of a plurality of time-of-day periods is an average count value for each of a plurality of time-of-day periods.

Clause 276. The system of any of clauses 272-275, wherein the summary graphic is a circular graphic, and wherein the total daily count value during the time period and the total count goal for the day are displayed in a center of the circular graphic.

Clause 277. The system of clause 276, wherein each of the aggregate count values for each of a plurality of time-of-day periods during the time period are displayed in a different portion of the circular graphic.

Clause 278. The system of any of clauses 276-277, wherein a portion of the circular graphic corresponding to a time-of-day period with a highest aggregate count value is a different color than a rest of the circular graphic.

Clause 279. The system of any of clauses 276-278, wherein the time-of-day periods are arranged clockwise in the circular graphic.

Clause 280. The system of any of clauses 272-279, wherein the summary graphic is a bar graph, and wherein bars corresponding to days having the aggregate total daily count higher than the total count goal comprise a first color and bars corresponding to days having the aggregate total daily count lower than or equal to the total count goal comprise a second color.

Clause 281. The system of any of clauses 272-280, wherein the instructions, when executed by the one or more processors, further cause the system to:
display a summary for each day of the time period.

Clause 282. The system of clause 281, wherein the summary for each day comprises a count total and a graphic highlighting a time-of-day period with a highest count value.

Clause 283. The system of any of clauses 272-282, wherein the instructions, when executed by the one or more processors, further cause the system to:
display a comparison of the determined plurality of glucose metrics to a plurality of glucose metrics for a population.

Clause 284. The system of clause 283, wherein the population is related to an age of a user.

Clause 285. The system of any of clauses 272-284, wherein the instructions, when executed by the one or more processors, further cause the system to:
display a recommended time-of-day period for a user to mitigate.

Clause 286. The system of any of clauses 272-285, wherein the instructions, when executed by the one or more processors, further cause the system to:
determine a new total count goal based on the assigned count value for each glucose metric of the plurality of glucose metrics in the time period; and
display the new total count goal.

Clause 287. The system of any of clauses 272-286, wherein the instructions, when executed by the one or more processors, further cause the system to:
assign a glycemic profile from a plurality of glycemic profiles based on the determined aggregate count value for each of the plurality of time of day periods; and
display the assigned glycemic profile.

Clause 288. The system of any of clauses 272-287, wherein the time period is one week, and wherein the instructions, when executed by the one or more processors, further cause the system to:
display a plurality of graphic elements corresponding to each day of the time period, wherein each of the plurality of graphic elements comprises the aggregate total daily count value determined for each day of the time period.

Clause 289. The system of clause 288, wherein a graphic element of the plurality of graphic elements having an aggregate total daily count value equal to or below the total count goal for the day is visually distinguishable from a graphic element of the plurality of graphic elements having an aggregate total daily count value above the total count goal for the day.

Clause 290. The system of any of clauses 272-289, wherein the instructions, when executed by the one or more processors, further cause the system to:
display a graph comprising the determined aggregate total daily count value during the time period and at least one aggregate daily count value determined from an earlier time period.

Clause 291. The system of any of clauses 272-290, wherein the instructions, when executed by the one or more processors, further cause the system to:
display a focus area identified by the user.

Clause 292. The system of any of clauses 272-291, wherein the input is configured to receive data related to a focus area identified by the user, an wherein the instructions, when executed by the one or more processors, further cause the system to:
display a graph of answers from a user prompt related to the focus area identified by the user.

Clause 293. The system of clause 292, wherein the graph is a bar graph.

Clause 294. The system of any of clauses 272-293, wherein the instructions, when executed by the one or more processors, further cause the system to:
display a graphic comprising the total count goal for the day for the time period and at least one total count goal for the day for a previous time period.

Clause 295. A system for determining metrics relating to a subject, the system comprising:
an input configured to receive measured glucose data;
a display configured to visually present information; and
one or more processors coupled with the input, the display, and a memory storing instructions that, when executed by the one or more processors, cause the system to:
determine a plurality of glucose metrics for each of a plurality of glucose spikes in a graph of glucose data vs. time over a time period;
   determine if each of the plurality of glucose spikes relates to a food event or relates to a non-food event;
   assign a count value for each glucose metric of the plurality of glucose metrics based on a comparison to a distribution of the glucose metric determined from a predetermined population; and
   calculate a first running sum of count values for each glucose metric determined for each of the plurality of glucose spikes related to a food event.

Clause 296. The system of clause 295, wherein the instructions, when executed by the one or more processors, further cause the system to:
calculate a second running sum of count values for each glucose metric determined for each of the plurality of glucose spikes related to a non-food event.

Clause 297. The system of any of clauses 295-296, wherein the instructions, when executed by the one or more processors, further cause the system to:
output the second running sum of count values on a display.

Clause 298. The system of any of clauses 295-297, wherein the instructions, when executed by the one or more processors, further cause the system to:
output the first running sum of count values on a display.

Clause 299. The system of any of clauses 295-298, wherein the plurality of glucose metrics comprises a plurality of calculated integrated areas under the curve over time for each of the plurality of glucose spikes.

Clause 300. A system for determining metrics relating to a subject, the system comprising:
an input configured to receive measured glucose data;
a display configured to visually present information; and
one or more processors coupled with the input, the display, and a memory storing instructions that, when executed by the one or more processors, cause the system to:
determine a plurality of glucose metrics for each of a plurality of glucose spikes in a graph of glucose data vs. time in each day of a first time period;
   assign a count value for each glucose metric of the plurality of glucose metrics based on a comparison to a distribution of the glucose metric determined from a predetermined population;
   determine an aggregate daily total count value for the first time period based on the assigned count values for each glucose metric of the plurality of glucose metrics; and
   determine a target daily count goal for a user for a second time period based on the determined aggregate daily total count value for the first time period.

Clause 301. The system of clause 300, wherein the target daily count goal for the user for the second time period is determined based on a comparison of the determined aggregate daily total count value for the first time period to a population of users having an aggregate daily total count value within a threshold of the determined aggregate daily total count value of the user.

Clause 302. The system of any of clauses 300-301, wherein the first time period comprises a first week.

Clause 303. The system of clause 302, wherein the second time period comprises a second week, wherein the second week occurs after the first week.

Clause 304. The system of clause 303, wherein the first week and second week are consecutive.

Clause 305. A system for monitoring and/or displaying metrics relating to glucose management of a user, the system comprising:
an input configured to receive time-correlated measured glucose data and data related answers from at least one prompt to a user;
a display configured to visually present information; and
one or more processors coupled with the input, the display, and a memory storing instructions that, when executed by the one or more processors, cause the system to:
determine a plurality of glucose metrics for each of a plurality of glucose spikes in a graph of glucose data vs. time in a time period;
   assign a count value for each glucose metric of the plurality of glucose metrics in the time period;
   determine a total count value for at least one day of the time period;
   determine a daily target count goal for the time period;
   display a plurality of graphic elements corresponding to each day of the time period, wherein each of the plurality of graphic elements comprises the total count value determined for the at least one day of the time period.

Clause 306. The system of clause 305, wherein the count value is assigned based on a comparison to a distribution of the glucose metric determined from a predetermined population.

Clause 307. The system of any of clauses 305-306, wherein the time period is one week.

Clause 308. The system of any of clauses 305-307, wherein a graphic element of the plurality of graphic elements corresponding to a first day of the time period comprises a total daily count value determined for the first day.

Clause 309. The system of any of clauses 305-308, wherein a graphic element of the plurality of graphic elements having a total daily count value equal to or below the daily target count goal is visually distinguishable from a graphic element of the plurality of graphic elements having a total daily count value above the daily target count goal.

Clause 310. The system of any of clauses 305-309, wherein the instructions, when executed by the one or more processors, further cause the system to:
display an indication of a focus area for the user.

Clause 311. The system of clause 310, wherein the focus area is selected from the group consisting of boost energy, manage hunger, improve mood, better sleep, and stay focused.

Clause 312. The system of any of clauses 305-311, wherein the daily target count goal is determined based on a comparison to a distribution of the counts determined for a predetermined population.

Clause 313. The system of clause 312, wherein the predetermined population is determined according to an age of the user.

Clause 314. The system of any of clauses 305-313, wherein the daily target count goal is determined based on a total count value determined for at least one day of a previous time period.

Clause 315. The system of any of clauses 305-314, wherein the instructions, when executed by the one or more processors, further cause the system to:
display a recommended time-of-day period for a user to mitigate.

Clause 316. The system of any of clauses 305-315, wherein the instructions, when executed by the one or more processors, further cause the system to:
determine an aggregate total daily count value for the time period and an aggregate total daily count value for at least one previous time period;
display a graph comprising the aggregate total daily count value for the time period and the aggregate total daily count value for the at least one previous time period.

Clause 317. The system of clause 316, wherein the aggregate total daily count value for the time period comprises the average total daily count value for the time period, and wherein the aggregate total daily count value for the at least one previous time period comprises the average total daily count value for the at least one previous time period.

Clause 318. The system of any of clauses 305-317, wherein the glucose management comprises at least a first stage and a second stage, wherein advancement of the user from the first stage to the second stage requires the user to satisfy at least one requirement, wherein the instructions, when executed by the one or more processors, further cause the system to:
display at least one progress indicator relating to the satisfaction of the at least one requirement.

Clause 319. The system of clause 318, wherein the at least one requirement comprises the user having a total daily count value equal to or below the daily target count goal for a minimum number of days in the time period.

Clause 320. The system of clause 319, wherein the minimum number of days is about 5 days and the time period is about one week.

Clause 321. The system of any of clauses 318-319, wherein the at least one requirement further comprises the user having a total daily count value equal to or below the daily target count goal for about 5 days in a one week time period for at least two weeks.

1-52. (canceled)

53. A system for monitoring metrics relating to a user, the system comprising:
wireless communications circuitry configured to receive time-correlated measured glucose data;
a display configured to visually present information; and
one or more processors coupled with the wireless communications circuitry, the display, and a memory storing instructions that, when executed by the one or more processors, cause the system to:
assign a count value for each glucose episode based at least on an area under a curve of the each glucose episode in a dataset of time-correlated glucose data;
calculate a running sum of count values for a plurality of glucose episodes in a time period; and
display a progress indicator representative of the running sum of the count values relative to a target count goal for the time period.

54. The system of claim 53, wherein the count value is assigned based on a comparison to a distribution of a glucose metric determined from a predetermined population.

55. The system of claim 53, wherein the dataset comprises a graph of glucose data vs. time.

56. The system of claim 53, wherein the time period is about one day.

57. The system of claim 53, wherein the progress indicator comprises a display of a fraction comprising the running sum of count values in a numerator and the target count goal in a denominator.

58. The system of claim 57, wherein the display of the fraction has a first end, a second end, and a length, and wherein a numerical value of the running sum of count values is displayed at a position along the length of the numerator that is proportional to [the running sum of count values]/[the target count goal] if the running sum of count values is less than or equal to the target count goal.

59. The system of claim 58, wherein the target count goal is displayed in the denominator near the second end.

60. The system of claim 57, wherein the display of the fraction has a first end, a second end, and a length, and wherein a numerical value of the target count goal is displayed at a position along the length of the denominator that is proportional to [the target count goal]/[the running sum of count values] if the running sum of count values is greater than the target count goal.

61. The system of claim 60, wherein a numerical value of the running sum of count values is displayed at the first end.

62. The system of claim 58, wherein when the numerical value of the running sum of count values is zero, the numerical value of the running sum of count values is displayed near the first end.

63. The system of claim 53, wherein the instructions, when executed by the one or more processors, further cause the system to:
determine a difference between a first count value assigned in a first time period and a second count value assigned in a second time period, wherein the second time period is immediately after the first time period;
assign a count trend status for the second time period from a plurality of count trend statuses based on the determined difference; and
display a color representative of the assigned count trend status for the second time period.

64. The system of claim 63, wherein the plurality of count trend statuses comprises a balanced status, a declining in spike status, a flat during spike status, and a rising in spike status.

65. The system of claim 63, wherein the first time period and the second time period both occur during a single glucose episode.

66. The system of claim 63, wherein the progress indicator is displayed in a graphic user interface (GUI), and wherein the color is displayed as a background color of the GUI.

67. The system of claim 63, wherein the color representative of the assigned count trend status is displayed as a background color of a graphic user interface (GUI).

68. The system of claim 63, wherein the instructions, when executed by the one or more processors, further cause the system to:
determine a difference between at least one additional count value in at least one additional time period and the second count value, wherein the at least one additional time period is immediately after the second time period;
assign a count trend status for the at least one additional time period from a plurality of count trend statuses based on the determined difference; and
display a color representative of the assigned count trend status for the at least one additional time period.

69. The system of claim 68, wherein the display comprises a graphic user interface, and wherein the color representative of the assigned count trend status for the at least one additional time period is displayed as a background color of a graphic user interface (GUI).

70. The system of claim 69, wherein the color representative of the assigned count trend status for the at least one additional time period is displayed as a background color of a first portion of the GUI, and the color representative of the assigned count trend status for the second time period is displayed in a second portion of the GUI.

71. The system of claim 70, wherein the first portion of the GUI is a top portion and wherein the second portion of the GUI is a bottom portion.

72. The system of claim 70, wherein the color representative of the assigned count trend status for the at least one additional time period and the color representative of the assigned count trend status for the second time period are displayed as blended colors.

73. (canceled)

74. The system of claim 53, wherein the instructions, when executed by the one or more processors, further cause the system to:
determine a slope for a line formed by count values determined for a plurality of periods of time;
assign a count trend status for at least one of the plurality of periods of time based on the determined slope; and
display a color representative of the assigned count trend status for the second time period.

75-201. (canceled)
